(19) United States
(12) Patent Application Publication
(10) Pub. No.: US 2026/0265208 A1
(43) Pub. Date: Sep. 10, 2026

(54) TETRALINE, PHENYLCYCLOBUTANE AND PHENYLCYCLOPENTANE ANALOGS AS RXFP1 AGONISTS

(71) Applicant: BRISTOL-MYERS SQUIBB COMPANY, Princeton, NJ (US)

(72) Inventors: Leon M. Smith, II, Somerset, NJ (US); Donald J.P. Pinto, Pennsylvania, PA (US); Michael J. Orwat, NEW Hope, PA (US)

(21) Appl. No.: 18/718,464

(22) PCT Filed: Dec. 14, 2022
(86) PCT No.: PCT/US2022/081516

§ 371 Date: Jun. 11, 2024

Related U.S. Application Data
(60) Provisional application No. 63/289,812, filed on Dec. 15, 2021.

(51) Int. Cl. C07D 275/02; A61K 31/167; A61K 31/196; A61K 31/198; A61K 31/27; A61K 31/351; A61K 31/415; A61K 31/423; A61K 31/424; A61K 31/425; C07C 237/48; C07C 271/24; C07C 271/28; C07D 231/12; C07D 261/08; C07D 261/20; C07D 309/08; C07D 498/04

(52) U.S. Cl. CPC C07D 275/02; A61K 31/167; A61K 31/196; A61K 31/198; A61K 31/27; A61K 31/351; A61K 31/415; A61K 31/423; A61K 31/424; A61K 31/425; C07C 237/48; C07C 271/24; C07C 271/28; C07D 231/12; C07D 261/08; C07D 261/20; C07D 309/08; C07D 498/04; C07C 2602/06; C07C 2602/08; C07C 2602/10

(57) ABSTRACT

The disclosure relates to compounds of Formula (I), which are RXFP1 receptor agonists, compositions containing them, and methods of using them, for example, in the treatment of heart failure, fibrotic diseases, and related diseases such as lung disease (e.g., idiopathic pulmonary fibrosis), kidney disease (e.g., chronic kidney disease), or hepatic disease (e.g., non-alcoholic steatohepatitis and portal hypertension).

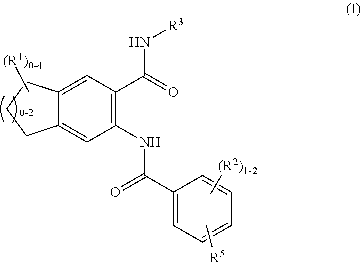

### CROSS-REFERENCE TO RELATED APPLICATIONS

This application claims the benefit of U.S. Provisional Application No. 63/289,812, filed Dec. 15, 2021, the disclosure of which is incorporated herein by reference in its entirety.

### BACKGROUND OF THE INVENTION

The present disclosure relates to novel compounds which are relaxin family peptide receptor 1 (RXFP1) agonists, compositions containing them, and methods of using them, for example in the treatment of heart failure, fibrotic diseases, and related diseases such as lung disease (e.g., idiopathic pulmonary fibrosis), kidney disease (e.g., chronic kidney disease), and hepatic disease (e.g., non-alcoholic steatohepatitis and portal hypertension).

The human relaxin hormone (also called relaxin or H2 relaxin) is a 6-kDa peptide composed of 53 amino acids whose activity was initially discovered when Frederick Hisaw in 1926 injected crude extracts from swine corpus luteum into virgin guinea pigs and observed a relaxation of the fibrocartilaginous pubic symphysis joint (Hisaw F L., Proc. Soc. Exp. Biol. Med., 1926, 23, 661-663). The relaxin receptor was previously known as Lgr7 but is now officially termed the relaxin family peptide receptor 1 (RXFP1) and was deorphanized as a receptor for relaxin in 2002 (Hsu S Y., et al., Science. 2002, 295, 671-674). RXFP1 is reasonably well conserved between mouse and human with 85% amino acid identity and is essentially ubiquitously expressed in humans and in other species (Halls M L., et al., Br. J. Pharmacol. 2007, 150, 677-691). The cell signaling pathways for relaxin and RXFP1 are cell type dependent and quite complex (Halls M L., et al., Br. J. Pharmacol., 2007, 150, 677-691; Halls M L., et al. Ann. N Y Acad. Sci., 2009, 1160, 108-111; Halls M L., Ann N Y Acad Sci., 2007, 1160, 117-120). The best studied pathway is the relaxin-dependent increase in cellular levels of cAMP in which relaxin functions as an RXFP1 agonist to promote GαS coupling and activation of adenylate cyclase (Halls M L., et al., Mol. Pharmacol., 2006, 70, 214-226).

Since the initial discovery of relaxin much experimental work has focused on delineating the role relaxin has played in female reproductive biology and the physiological changes that occur during mammalian pregnancy (Sherwood O D., Endocr. Rev., 2004, 25, 205-234). During human gestation, in order to meet the nutritional demands imposed upon it by the fetus, the female body undergoes a significant ~30% decrease in systemic vascular resistance (SVR) and a concomitant ~50% increase in cardiac output (Jeyabalan A C., K. P., Reanl and Electrolyte Disorders. 2010, 462-518), (Clapp J F. & Capeless E., Am. J. Cardio., 1997, 80, 1469-1473). Additional vascular adaptations include an ~30% increase in global arterial compliance that is important for maintaining efficient ventricular-arterial coupling, as well as an ~50% increase in both renal blood flow (RBF) and glomerular filtration rate (GFR), important for metabolic waste elimination (Jeyabalan A C., K. P., Reanl and Electolyte Disorders. 2010, 462-518), (Poppas A., et al., Circ., 1997, 95, 2407-2415). Both pre-clinical studies in rodents as well as clinical studies performed in a variety of patient settings, provide evidence that relaxin is involved, at least to some extent, in mediating these adaptive physiological changes (Conrad K P., Regul. Integr. Comp. Physiol., 2011, 301, R267-275), (Teichman S L., et al., Heart Fail. Rev., 2009, 14, 321-329). Importantly, many of these adaptive responses would likely be of benefit to HF patients in that excessive fibrosis, poor arterial compliance, and poor renal function are all characteristics common to heart failure patients (Mohammed S F., et al., Circ., 2015, 131, 550-559), (Wohlfahrt P., et al., Eur. J. Heart Fail., 2015, 17, 27-34), (Damman K., et al., Prog. Cardiovasc. Dis., 2011, 54, 144-153).

Heart failure (HF), defined hemodynamically as “systemic perfusion inadequate to meet the body's metabolic demands as a result of impaired cardiac pump function”, represents a tremendous burden on today's health care system with an estimated United States prevalence of 5.8 million and greater than 23 million worldwide (Roger V L., et al., Circ. Res., 2013, 113, 646-659). It is estimated that by 2030, an additional 3 million people in the United States alone will have HF, a 25% increase from 2010. The estimated direct costs (2008 dollars) associated with HF for 2010 was $25 billion, projected to grow to $78 B by 2030 (Heidenreich P A., et al., Circ., 2011, 123, 933-944). Astoundingly, in the United States, 1 in 9 deaths has HF mentioned on the death certificate (Roger V L., et al., Circ., 2012, 125, e2-220) and, while survival after HF diagnosis has improved over time (Matsushita K., et al., Diabetes, 2010, 59, 2020-2026), (Roger V L., et al., JAMA, 2004, 292, 344-350), the death rate remains high with ~50% of people with HF dying within 5 years of diagnosis (Roger V L., et al., Circ., 2012, 125, e2-220), (Roger V L., et al., JAMA, 2004, 292, 344-350).

The symptoms of HF are the result of inadequate cardiac output and can be quite debilitating depending upon the advanced stage of the disease. Major symptoms and signs of HF include: 1) dyspnea (difficulty in breathing) resulting from pulmonary edema due to ineffective forward flow from the left ventricle and increased pressure in the pulmonary capillary bed; 2) lower extremity edema occurs when the right ventricle is unable to accommodate systemic venous return; and 3) fatigue due to the failing heart's inability to sustain sufficient cardiac output (CO) to meet the body's metabolic needs (Kemp C D., & Conte J V., Cardiovasc. Pathol., 2011, 21, 365-371). Also, related to the severity of symptoms, HF patients are often described as “compensated” or “decompensated”. In compensated heart failure, symptoms are stable, and many overt features of fluid retention and pulmonary edema are absent. Decompensated heart failure refers to a deterioration, which may present as an acute episode of pulmonary edema, a reduction in exercise tolerance, and increasing breathlessness upon exertion (Millane T., et al., BMJ, 2000, 320, 559-562).

In contrast to the simplistic definition of poor cardiac performance not being able to meet metabolic demands, the large number of contributory diseases, multitude of risk factors, and the many pathological changes that ultimately lead to heart failure make this disease exceedingly complex (Jessup M. & Brozena S., N. Engl. J. Med., 2003, 348, 3007-2018). Injurious events thought to be involved in the pathophysiology of HF range from the very acute such as myocardial infarction to a more chronic insult such as life-long hypertension. Historically, HF was primarily described as “systolic HF” in which decreased left-ventricular (LV) contractile function limits the expulsion of blood and hence results in a reduced ejection fraction (EF is stroke volume/end diastolic volume), or “diastolic HF” in which active relaxation is decreased and passive stiffness is increased limiting LV filling during diastole, however overall EF is maintained (Borlaug B A. & Paulus W J., Eur Heart J., 2011, 32, 670-679). More recently, as it became understood that diastolic and systolic LV dysfunction was not uniquely specific to these two groups, new terminology was employed: “heart failure with reduced ejection fraction” (HFrEF), and “heart failure with preserved ejection fraction” (HFpEF) (Borlaug B A. & Paulus W J., Eur Heart J., 2011, 32, 670-679). Although these two patient populations have very similar signs and symptoms, whether HFrEF and HFpEF represent two distinct forms of HF or two extremes of a single spectrum sharing a common pathogenesis is currently under debate within the cardiovascular community (Borlaug B A. & Redfield M M., Circ., 2011, 123, 2006-2013), (De Keulenaer G W., & Brutsaert D L., Circ., 2011, 123, 1996-2004).

Serelaxin, an intravenous (IV) formulation of the recombinant human relaxin peptide with a relatively short first-phase pharmacokinetic half-life of 0.09 hours, is currently being developed for the treatment of HF (Novartis, 2014). Serelaxin has been given to normal healthy volunteers (NHV) and demonstrated to increase RBF (Smith M C., et al., J Am. Soc. Nephrol. 2006, 17, 3192-3197) and estimated GFR (Dahlke M., et al., J. Clin. Pharmacol., 2015, 55, 415-422). Increases in RBF were also observed in stable compensated HF patients (Voors A A., et al., Cir. Heart Fail., 2014, 7, 994-1002).

In large clinical studies, favorable changes in worsening renal function, worsening HF, as well as fewer deaths, were observed in acute decompensated HF (ADHF) patients in response to an in-hospital 48 hour IV infusion of serelaxin (Teerlink J R., et al., Lancet. 2013, 381, 29-39), (Ponikowski P., et al., Eur. Heart, 2014, 35, 431-441). Suggesting that chronic dosing of serelaxin could provide sustained benefit to HF patients, improvement in renal function based on serum creatinine levels was observed in scleroderma patients given serelaxin continuously for 6 months using a subcutaneous pump (Teichman S L., et al., Heart Fail. Rev., 2009, 14, 321-329). In addition to its potential as a therapeutic agent for the treatment of HF continuous subcutaneous administration of relaxin has also been demonstrated to be efficacious in a variety of animal models of lung (Unemori E N., et al., J. Clin. Invet., 1996, 98, 2739-2745), kidney (Garber S L., et al., Kidney Int., 2001, 59, 876-882), and liver injury (Bennett R G., Liver Int., 2014, 34, 416-426).

In summary, a large body of evidence supports a role for relaxin-dependent agonism of RXFP1 mediating the adaptive changes that occur during mammalian pregnancy, and that these changes translate into favorable physiological effects and outcomes when relaxin is given to HF patients. Additional preclinical animal studies in various disease models of lung, kidney, and liver injury provide evidence that relaxin, when chronically administered, has the potential to provide therapeutic benefit for multiple indications in addition to HF. More specifically, chronic relaxin administration could be of benefit to patients suffering from lung disease (e.g., idiopathic pulmonary fibrosis), kidney disease (e.g., chronic kidney disease), or hepatic disease (e.g., non-alcoholic steatohepatitis and portal hypertension).

### SUMMARY OF THE INVENTION

The present invention provides novel substituted tetraline, phenylcyclobutane and phenylcyclopentane compounds, their analogues, including stereoisomers, tautomers, pharmaceutically acceptable salts, or solvates thereof, which are useful as RXFP1 receptor agonists.

The present invention also provides processes and intermediates for making the compounds of the present invention.

The present invention also provides pharmaceutical compositions comprising a pharmaceutically acceptable carrier and at least one of the compounds of the present invention or stereoisomers, tautomers, pharmaceutically acceptable salts, or solvates thereof.

The compounds of the invention may be used, for example, in the treatment and/or prophylaxis of heart failure, fibrotic diseases, and related diseases, such as; lung disease (e.g., idiopathic pulmonary fibrosis), kidney disease (e.g., chronic kidney disease), or hepatic disease (e.g., non-alcoholic steatohepatitis and portal hypertension).

The compounds of the present invention may be used in therapy.

The compounds of the present invention may be used for the manufacture of a medicament for the treatment and/or prophylaxis of heart failure.

The compounds of the invention can be used alone, in combination with other compounds of the present invention, or in combination with one or more, preferably one to two other agent(s).

These and other features of the invention will be set forth in expanded form as the disclosure continues.

### DESCRIPTION OF THE INVENTION

The invention encompasses compounds of Formula (I), which are RXFP1 receptor agonists, compositions containing them, and methods of using them.

In a first aspect, the present invention provides, inter alia, compounds of Formula (I):

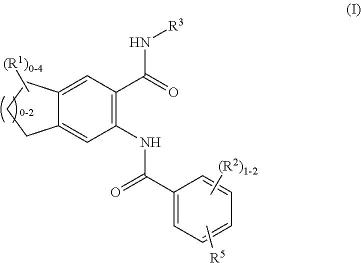

or a pharmaceutically acceptable salt thereof, wherein:
R¹ is halo, C₁₋₄ alkyl substituted with 0-5 halo, ═O, OH, or —OC₁₋₄ alkyl substituted with 0-5 halo;
R² is halo, CN, C₁₋₄ alkyl substituted with 0-5 halo or OH, or —OC₁₋₄ alkyl substituted with 0-5 halo, OH, or —OC₁₋₄ alkyl;
R³ is C₁₋₄ alkyl substituted with 0-5 R⁴, —(CR^{d}R^{d})ₙ, —C₃₋₁₀-carbocyclyl substituted with 0-5 R⁴ or —(CR^{d}R^{d})ₙ-3 to 6-membered heterocyclyl comprising 1-4 heteroatoms selected from O, S(═O)ₚ, N, and NR^{d}, and substituted with 0-5 R⁴;
R⁴ is halo, CN, C₁₋₄ alkyl substituted with 0-5 halo, OH, —OC₁₋₄ alkyl substituted with 0-5 halo, —S(O)ₚR^{c}, aryl, or a 4- to 6-membered heterocyclyl comprising 1-4 heteroatoms selected from O, S(═O)ₚ, N, and NR^{d};
R⁵ is aryl substituted with 0-3 R⁶ and 0-2 R⁷ or a 3- to 12-membered heterocyclyl comprising 1-5 heteroatoms selected from O, S(═O)ₚ, N and NR¹⁰, and substituted with 0-3 R⁶ and 0-2 R⁷; wherein said heterocyclyl is bonded to the phenyl moiety through a carbon or nitrogen atom;
R⁶ is halo, CN, ═O, —OH, —OC₁₋₄ alkyl, or C₁₋₄ alkyl substituted with 0-2 halo or OH;
R⁷ is C₁₋₄ alkyl substituted with 0-1 R⁸ and 0-1 R⁹, —OR, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, C₃₋₆ cycloalkyl, or a 4- to 6-membered heterocyclyl comprising 1-4 heteroatoms selected from O, S(═O)ₚ, N and NR^{d}, and substituted with 0-5 R^{e};
R⁸ is halo, —C(═O)OR, —C(═O)NR^{a}R^{a}, —C(═O)NR^{a}OR, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —NR^{a}S(O)ₚNR^{a}R^{a}, —OC(═O)NR^{a}R^{a}, —OC(═O)NR^{a}OR^{b}, —S(═O)ₚNR^{a}R^{a}, —S(O)ₚR^{c}, —(CH₂)ₙ—C₃₋₆ carbocyclyl substituted with 0-3 R^{e}, or —(CH₂)ₙ-heterocyclyl comprising 1-4 heteroatoms selected from O, S(═O)ₚ, and N, and substituted with 0-3 R^{e};
R¹⁰ is H, C₁₋₄ alkyl substituted with 0-4 R¹¹, —C(═O)R^{b}, —C(═O)OR^{b}, —C(═O)NR^{a}R^{a}, C₃₋₆ cycloalkyl substituted with 0-5 R^{e}, aryl substituted with 0-5 R^{e}, or a 4- to 6-membered heterocyclyl comprising 1-4 heteroatoms selected from O, S(═O)ₚ, N and NR², and substituted with 0-5 R^{e};
R¹¹ is —OH, —C(═O)OH, —C(═O)OC₁₋₄ alkyl, or aryl;
R¹² is H, C₁₋₄ alkyl, or aryl;
R^{a} is H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀ carbocyclyl substituted with 0-5 R^{e}, or —(CH₂)ₙ-heterocyclyl substituted with 0-5 R^{e}; or R^{a} and R^{a} together with the nitrogen atom to which they are both attached form a heterocyclyl substituted with 0-5 R^{e};
R^{b} is H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀carbocyclyl substituted with 0-5 R^{e}, or —(CH₂)ₙ-heterocyclyl substituted with 0-5 R^{e};
R^{c} is C₁₋₆ alkyl substituted with 0-5 R^{e}, C₂₋₆ alkenyl substituted with 0-5 R^{e}, C₂₋₆ alkynyl substituted with 0-5 R^{e}, C₃₋₆ carbocyclyl, or heterocyclyl;
R^{d} is H or C₁₋₄ alkyl;
R^{e} is halo, CN, NO₂, ═O, C₁₋₆ alkyl substituted with 0-5 R^{g}, C₂₋₆ alkenyl substituted with 0-5 R^{g}, C₂₋₆ alkynyl substituted with 0-5 R^{g}, —(CH₂)ₙC₃₋₆ cycloalkyl, —(CH₂)ₙ-aryl, —(CH₂)ₙ-heterocyclyl, —(CH₂)ₙOR^{f}, —C(═O)OR^{f}, —C(═O)NR^{f}R^{f}, —NR^{f}C(═O)R^{f}, —S(═O)ₚR^{f}, —S(═O)ₚNR^{f}R^{f}, —NRS(═O)ₚR^{f}, —NR^{f}C(═O)OR^{f}, —OC(═O)NR^{f}R^{f}, or —(CH₂)ₙNR^{f}R^{f};
R^{f} is H, C₁₋₆alkyl, C₃₋₆ cycloalkyl, aryl, or heterocyclyl; or R^{f} and R^{f} together with the nitrogen atom to which they are both attached form a heterocyclyl;
R^{g} is halo, CN, OH, C₁₋₆ alkyl, C₃₋₆ cycloalkyl, or aryl;
n is zero, 1, 2, or 3; and
p is zero, 1, or 2.

In a second aspect within the scope of the first aspect, the present invention provides compounds of Formula (II):

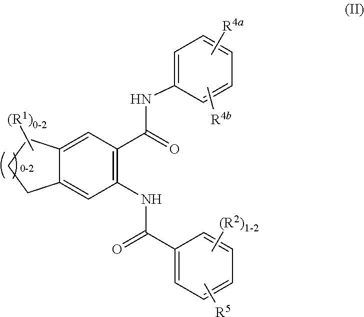

or pharmaceutically acceptable salts thereof, wherein:
R¹ is halo, ═O, OH, —OC₁₋₄ alkyl substituted with 0-5 halo;
R² is halo, C₁₋₃ alkyl, or —OC₁₋₃ alkyl substituted with 0-4 halo;
R^{4a} is halo;
R^{4b} is C₁₋₄ alkyl substituted with 0-4 halo:
R⁵ is C₆ aryl substituted with 0-3 R⁶ and 0-2 R⁷, or a 3- to 12-membered heterocyclyl comprising 1-4 heteroatoms selected from O, S(═O)ₚ, N and NR¹⁰ substituted with 0-3 R⁶ and 0-1 R⁷;
R⁷ is halo, ═O, —OH, —OC₁₋₄ alkyl, or C₁₋₄ alkyl substituted with 0-2 halo or OH;
R⁷ is C₁₋₃ alkyl substituted with 0-1 R⁸ and 0-1 R⁹, —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, C₃₋₆ cycloalkyl, or a 4- to 6-membered heterocyclyl comprising 1-4 heteroatoms selected from O, S(═O)ₚ, N and NR^{d}, and substituted with 0-5 R^{e};
R⁸ is halo, —C(═O)OR^{b}, —C(═O)NHR^{a}, —C(═O)NHOR^{b}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}S(═O)ₚR^{c}, —NR^{a}S(O)ₚNR^{a}R^{a}, —OC(═O)NR^{a}R^{a}, —OC(═O)NR^{a}OR^{b}, —S(═O)ₚNR^{a}R^{a}, or —S(O)ₚR^{c};
R¹⁰ is H, C₁₋₄ alkyl substituted with 0-2 R¹¹, —C(═O)R^{b}, —C(═O)OR^{b}, —C(═O)NR^{a}R^{a}, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e};
R¹¹ is —OH, —C(═O)OH, or aryl;
R¹² is H, C₁₋₃ alkyl, or aryl;
R^{a} is H, C₁₋₅ alkyl substituted with 0-5 R^{e}, C₂₋₅ alkenyl substituted with 0-5 R^{e}, C₂₋₅ alkynyl substituted with 0-5 R^{e}, —(CH₂)ₙ—C₃₋₁₀carbocyclyl substituted with 0-5 R^{e}, or —(CH₂)ₙ-heterocyclyl substituted with 0-5 R^{e}; or R^{a} and R^{a} together with the nitrogen atom to which they are both attached form a heterocyclyl substituted with 0-5 R^{e};
R^{b} is H, C₁₋₅ alkyl substituted with 0-5 R^{e}, C₂₋₃ alkenyl substituted with 0-5 R^{e}, C₂₋₅ alkynyl substituted with 0-5 R^{e}, —(CH₂)ₙ—C₃₋₁₀carbocyclyl substituted with 0-5 R^{e}, or —(CH₂)ₙ-heterocyclyl substituted with 0-5 R^{e};
R^{c} is C₁₋₅ alkyl substituted with 0-5 R^{e}, C₂₋₅ alkenyl substituted with 0-5 R^{e}, C₂₋₅ alkynyl substituted with 0-5 R^{e}, C₃₋₆ carbocyclyl, or heterocyclyl;
R^{d} is H or C₁₋₄ alkyl;
R^{e} is halo, CN, ═O, C₁₋₆ alkyl substituted with 0-5 R^{g}, C₂₋₆ alkenyl substituted with 0-5 R^{g}, C₂₋₆ alkynyl substituted with 0-5 R^{g}, —(CH₂)ₙ—C₃₋₆ cycloalkyl, —(CH₂)ₙ-aryl, —(CH₂)ₙ-heterocyclyl, —(CH₂)ₙOR, or —C(═O)OR^{f};
R^{f} is H or C₁₋₃ alkyl,
R^{g} is halo, CN, OH, C₁₋₆ alkyl, or C₃₋₆ cycloalkyl;
n is zero, 1, 2, or 3; and
p is zero, 1, or 2.

In a third aspect within the scope of the first and second aspects, the present invention provides compounds of Formula (III):

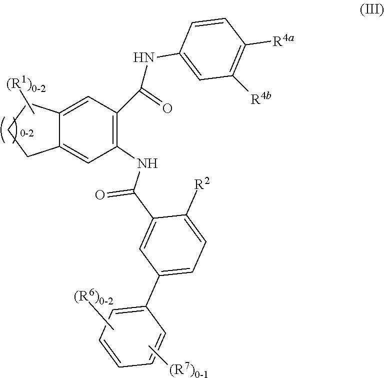

or pharmaceutically acceptable salts thereof, wherein:
R¹ is OH or ═O;
R² is —OC₁₋₄ alkyl substituted with 0-4 halo;
R^{4a} is halo;
R^{4b} is C₁₋₃ alkyl substituted with 0-4 F;
R⁶ is halo, CN, C₁₋₃ alkyl, —OH, or —OC₁₋₄ alkyl;
R⁷ is C₁₋₂ alkyl substituted with 0-1 R⁸ and 0-1 R⁹, OR^{b}, —NR^{a}R^{a}, —NR^{a}C(═O)R^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, C₃₋₆ cycloalkyl, or a 4- to 6-membered heterocyclyl comprising 1-4 heteroatoms selected from O, S(═O)ₚ, N and NR^{d}, and substituted with 0-4 R^{e};
R⁸ is halo, —C(═O)OR^{b}, —C(═O)NHR^{a}, —C(═O)NHOR^{b}, or C₁₋₄ alkyl substituted with 0-3 halo or OH:
R⁹ is —OR, —NR^{a}R^{a}, —NR^{a}C(═O)R^{b}, —NR^{a}C(═O)OR^{b}, —NR^{a}S(═O)ₚR^{c}, —NR^{a}S(O)ₚNR^{a}R^{a}, —OC(═O)NR^{a}R^{a}, —OC(═O)NR^{a}OR^{b}, —S(═O)ₚNR^{a}R^{a}, or —S(O)ₚR^{c};
R^{a} is H, C₁₋₅ alkyl substituted with 0-4 R^{e}, C₂₋₅ alkenyl substituted with 0-4 R^{e}, C₂₋₅ alkynyl substituted with 0-4 R^{e}, —(CH₂)ₙ—C₃₋₁₀ carbocyclyl substituted with 0-4 R^{e}, or —(CH₂)ₙ-heterocyclyl substituted with 0-4 R^{e}; or R^{a} and R^{a} together with the nitrogen atom to which they are both attached form a heterocyclyl substituted with 0-4 R^{e};
R^{b} is H, C₁₋₅ alkyl substituted with 0-4 R^{e}, C₂₋₅ alkenyl substituted with 0-4 R^{e}, C₂₋₅ alkynyl substituted with 0-4 R^{e}, —(CH₂)ₙ—C₃₋₁₀ carbocyclyl substituted with 0-4 R^{e}, or —(CH₂)ₙ-heterocyclyl substituted with 0-4 R^{e};
R^{c} is C₁₋₅ alkyl substituted with 0-4 R^{e}, C₂₋₅ alkenyl substituted with 0-4 R^{e}, C₂₋₅ alkynyl substituted with 0-4 R^{e}, C₃ carbocyclyl, or heterocyclyl;
R^{d} is H or C₁₋₂ alkyl:
R^{e} is halo, CN, ═O, C₁₋₆ alkyl substituted with 0-5 R^{g}, C₂₋₆ alkenyl substituted with 0-5 R^{g}, C₂₋₆ alkynyl substituted with 0-5 R^{g}, —(CH₂)ₙ—C₃₋₆ cycloalkyl, —(CH₂)ₙ-aryl, —(CH₂)ₙ-heterocyclyl, —(CH₂)ₙOR, or —C(═O)OR^{f};
R^{f} is H or C₁₋₃ alkyl.
R^{g} is halo, CN, OH, C₁₋₆alkyl, or C₃₋₆ cycloalkyl;
n is zero, 1, 2, or 3; and
p is zero, 1, or 2.

In a fourth aspect within the scope of the first to third aspects, the present invention provides compounds of Formula (IV):

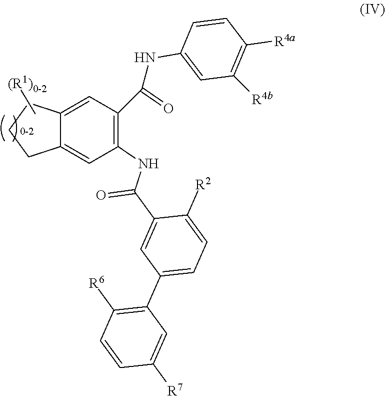

or pharmaceutically acceptable salts thereof, wherein:
R¹ is OH or ═O;
R² is —OC₁₋₃ alkyl;
R^{4a} is F;
R^{4b} is CF₃;
R⁶ is F;
R⁷ is C₁₋₂ alkyl substituted with 0-1 R⁸ and 0-1 R⁹, —C(═O)OR^{b}, or —C(═O)NR^{a}R^{a};
R⁸ is —C(═O)OR^{b}, —C(═O)NHR^{a}, or C₁₋₄ alkyl substituted with 0-3 halo or OH:
R⁹ is —OR^{b}, —NR^{a}R^{a}, —NR^{a}C(═O)R^{b}, or —OC(═O)NR^{a}R^{a};
R^{a} is H, C₁₋₄ alkyl substituted with 0-3 R^{e}, —(CH₂)ₙ—C₃₋₆ cycloalkyl substituted with 0-3 R^{e}, or phenyl, substituted with 0-3 R^{e};
R^{b} is H or heterocyclyl substituted with 0-3 R^{e};
R^{e} is halo, CN, ═O, or C₁₋₆ alkyl; and
n is zero or 1.

In a fifth aspect within the scope of the fourth aspect, the present invention provides compounds of Formula (V):

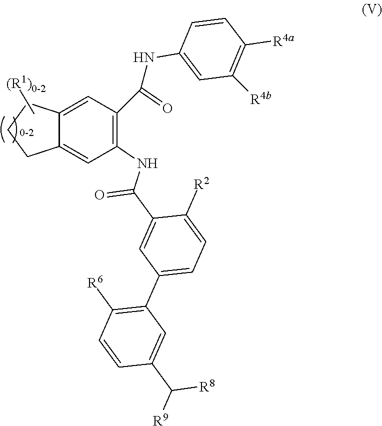

or pharmaceutically acceptable salts thereof, wherein:
R⁸ is —C(═O)OH or CF₃;
R⁹ is —NHC(═O)R^{b} or —OC(═O)NHR^{a};
R^{a} is —C₃₋₆ cycloalkyl or phenyl; and
R^{b} is heterocyclyl.

In a sixth aspect within the scope of the first and second aspects, the present invention provides compounds of Formula (VI):

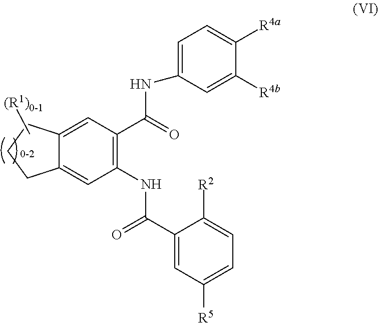

or pharmaceutically acceptable salts thereof, wherein:
R¹ is ═O;
R² is —OC₁₋₄ alkyl substituted with 0-4 halo;
R^{4a} is halo;
R^{4b} is C₁₋₃ alkyl substituted with 0-4 halo;
R⁵ is a 3- to 12-membered heterocyclyl comprising 1-4 heteroatoms selected from O, S(═O)ₚ, N and NR¹⁰ substituted with 0-3 R⁶ and 0-1 R⁷;
R⁶ is halo, ═O, —OH, —OC₁₋₄ alkyl, or C₁₋₄ alkyl substituted with 0-2 halo or OH;
R⁷ is C₁₋₂ alkyl substituted with 0-1 R⁸ and 0-1 R⁹, —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, C₃₋₆ cycloalkyl, or a 4- to 6-membered heterocyclyl comprising 1-4 heteroatoms selected from O, S(═O)ₚ, N and NR^{d}, and substituted with 0-4 R^{e};
R⁸ is —C(═O)OR^{b}, —C(═O)NHR^{a}, —C(═O)NHOR^{b}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}S(═O)ₚR^{c}, —NR^{a}S(O)ₚNR^{a}R^{a}, —OC(═O)NR^{a}R^{a}, —S(═O)ₚNR^{a}R^{a}, or —S(O)ₚR^{c};
R¹¹ is H, C₁₋₃ alkyl substituted with 0-2 R¹¹, —C(═O)R^{b}, —C(═O)OR^{b}, —C(═O)NR^{a}R^{a}, C₃₋₆ cycloalkyl, or a 4- to 6-membered heterocyclyl comprising 1-4 heteroatoms selected from O, S(═O)ₚ, N and NR¹², and substituted with 0-5 R^{e};
R¹¹ is —OH, —C(═O)OH, or aryl;
R¹² is H, C₁₋₄ alkyl, or phenyl;
R^{a} is H, C₁₋₅ alkyl substituted with 0-4 R^{e}, C₂₋₅ alkenyl substituted with 0-4R, C₂₋₅ alkynyl substituted with 0-4 R^{e}, —(CH₂)ₙ—C₃₋₁₀ carbocyclyl substituted with 0-4 R, or —(CH₂)ₙ-heterocyclyl substituted with 0-4 R^{e}; or R^{a} and R^{a} together with the nitrogen atom to which they are both attached form a heterocyclyl substituted with 0-4 R^{e};
R^{b} is H, C₁₋₅ alkyl substituted with 0-4 R^{e}, C₂₋₅ alkenyl substituted with 0-4 R^{e}, C₂₋₅ alkynyl substituted with 0-4 R^{e}, —(CH₂)ₙ—C₃₋₁₀ carbocyclyl substituted with 0-4 R^{e}, or —(CH₂)ₙ-heterocyclyl substituted with 0-4 R^{e};
R^{c} is C₁₋₅ alkyl substituted with 0-4 R^{e}, C₂₋₅ alkenyl substituted with 0-4 R^{e}, C₂₋₅ alkynyl substituted with 0-4 R^{e}, C₃₋₆carbocyclyl, or heterocyclyl;
R^{d} is H or C₁₋₂ alkyl;
R^{e} is halo, CN, ═O, C₁₋₆ alkyl substituted with 0-5 R^{g}, C₂₋₆ alkenyl substituted with 0-5 R^{g}, C₂₋₆ alkynyl substituted with 0-5 R^{g}, —(CH₂)ₙ—C₃₋₆ cycloalkyl, —(CH₂)ₙ-aryl, —(CH₂)ₙ-heterocyclyl, —(CH₂)ₙOR^{f}, or —C(═O)OR^{f};
R^{f} is H or C₁₋₃ alkyl,
R^{g} is halo, CN, OH, C₁₋₆alkyl, or C₃₋₆ cycloalkyl;
n is zero, 1, 2, or 3; and
p is zero, 1, or 2.

In a seventh aspect within the scope of the sixth aspect, the present invention provides compounds of Formula (VI) or pharmaceutically acceptable salts, thereof, wherein:
R² is —OCH₃;
R^{4a} is F;
R^{4b} is CF₃;
R⁵ is

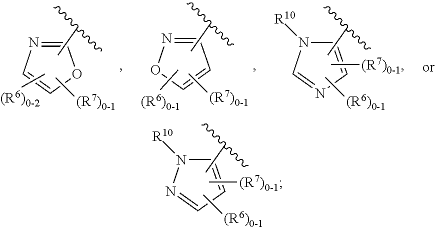

R⁶ is halo, —OH, or C₁₋₄ alkyl substituted with 0-1 OH:
R⁷ is C₁₋₂ alkyl substituted with 0-1 R⁸ and 0-1 R⁹;
R⁸ is —C(═O)OR^{b}, —C(═O)NHR^{a}, or —C(═O)NHOR^{b};
R⁹ is —OR^{b} or —NR^{a}R^{a};
R¹⁰ is H, —C(═O)R^{b}, or C₁₋₄ alkyl substituted with 0-1R¹¹;
R¹¹ is —OH, —C(═O)OH, or aryl;
R^{a} is H or C₁₋₃ alkyl; and
R^{b} is H or C₁₋₃ alkyl.

In an eighth aspect within the scope of the sixth aspect, the present invention provides compounds of Formula (VI) or pharmaceutically acceptable salts thereof,
wherein:
R² is —OCH₃;
R^{4a} is F;
R^{4b} is CF₃;
R⁵ is

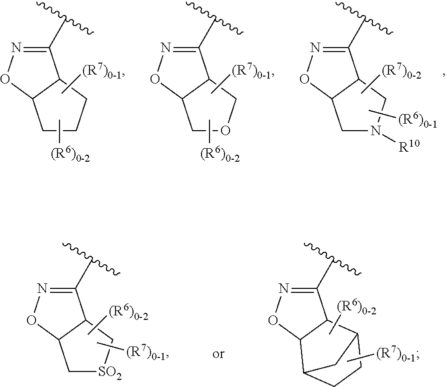

R⁶ is halo, C₁₋₄ alkyl, —OH, or —OC₁₋₄ alkyl;
R⁷ is C₁₋₄ alkyl substituted with 0-1 R⁸ and 0-1 R⁹;
R⁸ is —C(═O)OR^{b};
R⁹ is OH;
R¹⁰ is H, C₁₋₃ alkyl substituted with 0-2 R¹¹, or —C(═O)OC₁₋₄ alkyl;
R¹¹ is —OH, —C(═O)OH, or aryl; and
R^{b} is H or C₁₋₄ alkyl.

In a ninth aspect within the scope of the sixth aspect, the present invention provides compounds of Formula (VI), or pharmaceutically acceptable salts thereof,
wherein:
R² is —OCH₃;
R^{4a} is F;
R^{4b} is CF₃;
R⁵ is

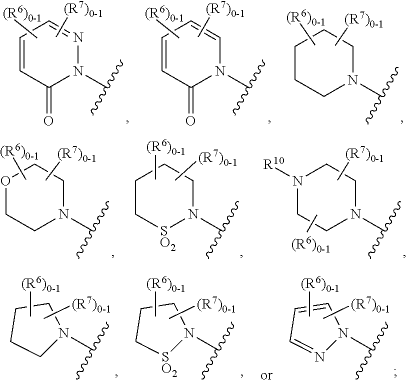

R⁶ is halo, CN, C₁₋₄ alkyl, ═O, —OH, or —OC₁₋₄ alkyl;
R⁷ is C₁₋₂ alkyl substituted with 0-1 Rand 0-1 R⁹, —NR^{a}R^{a}, —NR^{a}C(═O)R^{b}, —NR^{a}C(═O)OR^{b}, or —C(═O)OR^{b};
R⁸ is —C(═O)OR^{b}, —C(═O)NHR^{a}, —C(═O)NHOR^{b}, or C₁₋₄ alkyl substituted with 0-3 halo or OH;
R⁹ is —NR^{a}C(═O)R^{b};
R¹⁰ is H or C₁₋₃ alkyl:
R^{a} is H or C₁₋₄ alkyl; and
R^{b} is H or 2C₁₋₄ alkyl.

In a tenth aspect within the scope of the first aspect, the present invention provides compounds of Formula (VII):

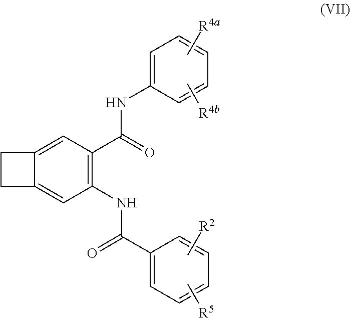

or pharmaceutically acceptable salts thereof, wherein:
R² is —OC₁₋₄ alkyl substituted with 0-4 halo, OH, or —OC₁₋₄ alkyl;
R^{4a} is halo;
R^{4b} is C₁₋₄ alkyl substituted with 0-4 halo;
R⁵ is C₆ aryl substituted with 0-3 R⁶ and 0-2 R⁷, or a 3- to 12-membered heterocyclyl comprising 1-4 heteroatoms selected from O, S(═O)ₚ, N and NR¹⁰ substituted with 0-3 R⁶ and 0-1 R⁷;
R⁶ is halo, ═O, —OH, —OC₁₋₄ alkyl, or C₁₋₄ alkyl substituted with 0-2 halo or OH;
R⁷ is C₁₋₃ alkyl substituted with 0-1 R⁸ and 0-1 R⁹, —OR^{b}, —NR^{a}R^{a}, —NR^{a}C(═O)R^{b}, —NR^{a}C(═O)OR^{b}, —NR^{a}C(═O)NR^{a}R^{a}, —NRS(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, C₃₋₆ cycloalkyl, or a 4- to 6-membered heterocyclyl comprising 1-4 heteroatoms selected from O, S(═O)ₚ, N and NR^{d}, and substituted with 0-5 R^{e};
R⁸ is halo, —C(═O)OR^{b}, —C(═O)NHR^{a}, —C(═O)NHOR^{b}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}S(═O)ₚR^{c}, —NR^{a}S(O)ₚNR^{a}R^{a}, —OC(═O)NR^{a}R^{a}, —OC(═O)NR^{a}OR^{b}, —S(═O))ₚNR^{a}R^{a}, or —S(O)ₚR^{c};
R¹⁰ is H, C₁₋₄ alkyl substituted with 0-2 R¹¹, —C(═O)R^{b}, —C(═O)OR^{b}, —C(═O)NR^{a}R^{a}, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e};
R¹¹ is —OH, —C(═O)OH, or aryl;
R¹² is H, C₁₋₃ alkyl, or aryl;
R^{a} is H, C₁₋₅ alkyl substituted with 0-5 R^{e}, C₂₋₅ alkenyl substituted with 0-5 R^{e}, C₂₋₅ alkynyl substituted with 0-5 R^{e}, —(CH₂)ₙ—C₃₋₁₀carbocyclyl substituted with 0-5 R^{e}, or —(CH₂)ₙ-heterocyclyl substituted with 0-5 R^{e}; or R^{a} and R^{a} together with the nitrogen atom to which they are both attached form a heterocyclyl substituted with 0-5 R^{e};
R^{b} is H, C₁₋₅ alkyl substituted with 0-5 R^{e}, C₂₋₅ alkenyl substituted with 0-5 R^{e}, C₂₋₅ alkynyl substituted with 0-5 R^{e}, —(CH₂)ₙ—C₃₋₁₀carbocyclyl substituted with 0-5 R^{e}, or —(CH₂)ₙ-heterocyclyl substituted with 0-5 R^{e};
R^{c} is C₁₋₃ alkyl substituted with 0-5 R^{e}, C₂₋₅ alkenyl substituted with 0-5 R^{e}, C₂₋₅ alkynyl substituted with 0-5 R^{e}, C₃₋₆ carbocyclyl, or heterocyclyl;
R^{d} is H or C₁₋₄ alkyl;
R^{e} is halo, CN, ═O, C₁₋₆ alkyl substituted with 0-5 R^{g}, C₂₋₆ alkenyl substituted with 0-5 R^{g}, C₂₋₆ alkynyl substituted with 0-5 R^{g}, —(CH₂)ₙ—C₃₋₆ cycloalkyl, —(CH₂)ₙ-aryl, —(CH₂)ₙ-heterocyclyl, —(CH₂)ₙOR^{f}, or —C(═O)OR^{f};
R^{f} is H or C₁₋₃ alkyl,
R^{g} is halo, CN, OH, C₁₋₆ alkyl, or C₃₋₆ cycloalkyl;
n is zero, 1, 2, or 3; and
p is zero, 1, or 2.

In an eleventh aspect within the scope of the first aspect, the present invention provides compounds of Formula (VIII):

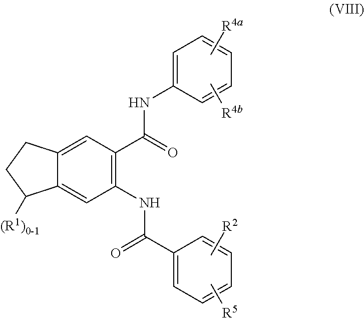

or pharmaceutically acceptable salts thereof, wherein:
R¹ is ═O or —OH;
R² is —OC₁₋₅ alkyl substituted with 0-4 halo, OH, or —OC₁₋₄ alkyl;
R^{4a} is halo;
R^{4b} is C₁₋₄ alkyl substituted with 0-4 halo;
R⁵ is C₆ aryl substituted with 0-3 R⁶ and 0-2 R⁷, or a 3- to 12-membered heterocyclyl comprising 1-4 heteroatoms selected from O, S(═O)ₚ, N and NR¹⁰ substituted with 0-3 R⁶ and 0-1 R⁷;
R⁶ is halo, ═O, —OH, —OC₁₋₄ alkyl, or C₁₋₄ alkyl substituted with 0-2 halo or OH;
R⁷ is C₁₋₃ alkyl substituted with 0-1 R⁸ and 0-1 R⁹, —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, C₃₋₆ cycloalkyl, or a 4- to 6-membered heterocyclyl comprising 1-4 heteroatoms selected from O, S(═O)ₚ, N and NR^{d}, and substituted with 0-5 R^{e};
R⁸ is halo, —C(═O)OR^{b}, —C(═O)NHR^{a}, —C(═O)NHOR^{b}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}S(═O)ₚR^{c}, —NR^{a}S(O)ₚNR^{a}R^{a}, —OC(═O)NR^{a}R^{a}, —OC(═O)NR^{a}OR^{b}, —S(═O)ₚNR^{a}R^{a}, or —S(O)ₚR^{c};
R¹⁰ is H, C₁₋₄ alkyl substituted with 0-2 R¹¹, —C(═O)R^{b}, —C(═O)OR^{b}, —C(═O)NR^{a}R^{a}, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e};
R¹¹ is —OH, —C(═O)OH, or aryl;
R¹² is H, C₁₋₃ alkyl, or aryl;
R^{a} is H, C₁₋₅ alkyl substituted with 0-5 R^{e}, C₂₋₅ alkenyl substituted with 0-5 R^{e}, C₂₋₅ alkynyl substituted with 0-5 R^{e}, —(CH₂)ₙ-C₃₋₁₀ carbocyclyl substituted with 0-5 R^{e}, or —(CH₂)ₙ-heterocyclyl substituted with 0-5 R^{e}; or R^{a} and R^{a} together with the nitrogen atom to which they are both attached form a heterocyclyl substituted with 0-5 R^{e};
R^{b} is H, C₁₋₅ alkyl substituted with 0-5 R^{e}, C₂₋₅ alkenyl substituted with 0-5 R^{e}, C₂₋₅ alkynyl substituted with 0-5 R^{e}, —(CH₂)ₙ—C₃₋₁₀carbocyclyl substituted with 0-5 R^{e}, or —(CH₂)ₙ-heterocyclyl substituted with 0-5 R^{e};
R^{c} is C₁₋₅ alkyl substituted with 0-5 R^{e}, C₂₋₅ alkenyl substituted with 0-5 R^{e}, C₂₋₅ alkynyl substituted with 0-5 R^{e}, C₃₋₆ carbocyclyl, or heterocyclyl;
R^{d} is H or C₁₋₄ alkyl;
R^{e} is halo, CN, ═O, C₁₋₆ alkyl substituted with 0-5 R^{g}, C₂₋₆ alkenyl substituted with 0-5 R^{g}, C₂₋₆ alkynyl substituted with 0-5 R^{g}, —(CH₂)ₙ—C₃₋₆ cycloalkyl, —(CH₂)ₙ-aryl, —(CH₂)ₙ-heterocyclyl, —(CH₂)ₙOR, or —C(═O)OR^{f};
R^{f} is H or C₁₋₃ alkyl,
R^{g} is halo, CN, OH, C₁₋₆ alkyl, or C₃₋₆ cycloalkyl;
n is zero, 1, 2, or 3; and
p is zero, 1, or 2.

In a twelfth aspect within the scope of the first aspect, the present invention provides compounds of Formula (IX):

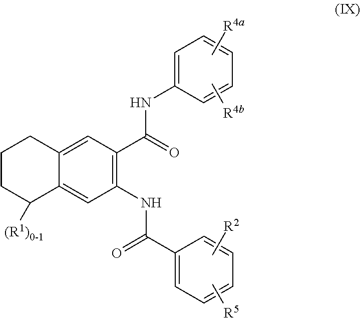

or pharmaceutically acceptable salts thereof, wherein:
R¹ is ═O or —OH;
R² is —OC₁₋₄ alkyl substituted with 0-4 halo, OH, or —OC₁₋₄ alkyl;
R^{4a} is halo;
R^{4b} is C₁₋₄ alkyl substituted with 0-4 halo;
R⁵ is C₆ aryl substituted with 0-3 R⁶ and 0-2 R⁷, or a 3- to 12-membered heterocyclyl comprising 1-4 heteroatoms selected from O, S(═O)ₚ, N and NR¹⁰ substituted with 0-3 R⁶ and 0-1 R⁷;
R⁶ is halo, ═O, —OH, —OC₁₋₄ alkyl, or C₁₋₄ alkyl substituted with 0-2 halo or OH;
R⁷ is C₁₋₃ alkyl substituted with 0-1 R⁸ and 0-1 R⁹. —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a} C₃₋₆ cycloalkyl, or a 4- to 6-membered heterocyclyl comprising 1-4 heteroatoms selected from O, S(═O)ₚ, N and NR^{d}, and substituted with 0-5 R^{e};
R⁸ is halo, —C(═O)OR^{b}, —C(═O)NHR^{a}, —C(═O)NHOR^{b}, or C₁₋₄ alkyl substituted with 0-3 halo or OH:
R⁹ is —OR^{b}, —NR^{a}R^{a}, —NR^{a}C(═O)R^{b}, —NR^{a}C(═O)OR^{b}, —NR^{a}S(═O)ₚR^{c}, —NR^{a}S(O)ₚNR^{a}R^{a}, —OC(═O)NR^{a}R^{a}, —OC(═O)NR^{a}OR^{b}, —S(═O)ₚNR^{a}R^{a}, or —S(O)ₚR^{c};
R¹⁰ is H, C₁₋₄ alkyl substituted with 0-2 R¹¹, —C(═O)R^{b}, —C(═O)OR^{b}, —C(═O)NR^{a}R^{a}, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e};
R¹¹ is —OH, —C(═O)OH, or aryl;
R¹² is H, C₁₋₃ alkyl, or aryl;
R^{a} is H, C₁₋₅ alkyl substituted with 0-5 R^{e}, C₂₋₅ alkenyl substituted with 0-5 R^{e}, C₂₋₅ alkynyl substituted with 0-5 R^{e}, —(CH₂)ₙ—C₃₋₁₀ carbocyclyl substituted with 0-5 R^{e}, or —(CH₂)ₙ-heterocyclyl substituted with 0-5 R^{e}; or R^{a} and R^{a} together with the nitrogen atom to which they are both attached form a heterocyclyl substituted with 0-5 R^{e};
R^{b} is H, C₁₋₅ alkyl substituted with 0-5 R^{e}, C₂₋₅ alkenyl substituted with 0-5 R^{e}, C₂₋₅ alkynyl substituted with 0-5 R^{e}, —(CH₂)ₙ—C₃₋₁₀carbocyclyl substituted with 0-5 R^{e}, or —(CH₂)ₙ-heterocyclyl substituted with 0-5 R^{e};
R^{c} is C₁₋₅ alkyl substituted with 0-5 R^{e}, C₂₋₅ alkenyl substituted with 0-5 R^{e}, C₂₋₅ alkynyl substituted with 0-5 R^{e}, C₃₋₆ carbocyclyl, or heterocyclyl;
R^{d} is H or C₁₋₄ alkyl;
R^{e} is halo, CN, ═O, C₁₋₆ alkyl substituted with 0-5 R^{g}, C₂₋₆ alkenyl substituted with 0-5 R^{g}, C₂₋₆ alkynyl substituted with 0-5 R^{g}, —(CH₂)ₙ—C₃₋₆ cycloalkyl, —(CH₂)ₙ-aryl, —(CH₂)ₙ-heterocyclyl, —(CH₂)ₙOR, or —C(═O)OR^{f};
R^{f} is H or C₁₋₃ alkyl,
R^{g} is halo, CN, OH, C₁₋₆ alkyl, or C₃₋₆ cycloalkyl;
n is zero, 1, 2, or 3; and
p is zero, 1, or 2.

For a compound of Formula (I), the scope of any instance of a variable substituent, including R¹, R², R³, R⁴ (R^{4a}, R^{4b}), R⁵, R⁶, R⁷, R⁸, R⁹, R¹⁰, R¹¹, R¹², R^{a}, R^{b}, R^{c}, R^{d}, R^{e}, R^{f}, and R^{g} can be used independently with the scope of any other instance of a variable substituent. As such, the invention includes combinations of the different aspects.

In one embodiment of Formula (II),

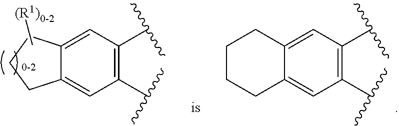

In another embodiment of Formula (II),

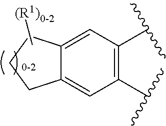

is

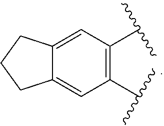 [SMILES: *c1cc2c(cc1*)CCC2]

In another embodiment of Formula (II),

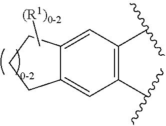

is

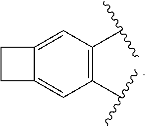 [SMILES: *c1cc2c(cc1*)CC2]

In another embodiment of Formula (II), R^{4a} is F.

In another embodiment of Formula (II), R^{4b} is CF₃.

In another embodiment of Formula (III),

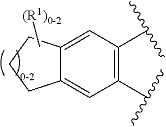

is

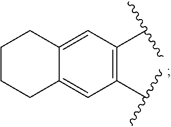 [SMILES: *c1cc2c(cc1*)CCCC2]

R² is —OCH₃; R^{4a} is F; R^{4b} is CF₃; R⁵ is

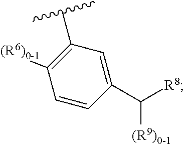

R⁶ is F; R⁸ is —C(═O)OH, —C(═O)NHR^{a}, or CF₃; R⁹ is —NHR^{a}, —NHC(═O)R^{b}, —NHS(═O)ₚC₁₋₄ alkyl or —OC(═O)NHR^{a}; R^{a} is H, C₁₋₃ alkyl, —(CH₂)₀₋₁—C₃₋₆ cycloalkyl, or —(CH₂)₀₋₁-phenyl substituted with 0-2 R^{e}; R^{b} is H or heterocyclyl; R^{e} is C₁₋₃ alkyl, —(CH₂)₀₋₁OR^{f}; and R^{f} is H or C₁₋₃ alkyl.

In another embodiment of Formula (III),

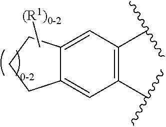

is

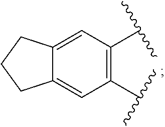 [SMILES: *c1cc2c(cc1*)CCC2]

R² is —OCH₃; R^{4a} is F; R^{4b} is CF₃; R⁵ is

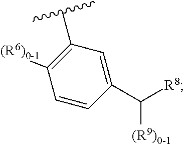

R⁶ is F; R⁸ is —C(═O)OH, —C(═O)NHR^{a}, or CF₃; R⁹ is —NHR^{a}, —NHC(═O)R^{b}, —NHS(═O)ₚC₁₋₄ alkyl or —OC(═O)NHR^{a}; R^{a} is H, C₁₋₃ alkyl, —(CH₂)₀₋₁—C₃₋₆ cycloalkyl, or —(CH₂)₀₋₁-phenyl substituted with 0-2 R^{e}; R^{b} is H or heterocyclyl; R^{e} is C₁₋₃ alkyl, —(CH₂)₀₋₁OR^{f}; and R^{f} is H or C₁₋₃ alkyl.

In another embodiment of Formula (III),

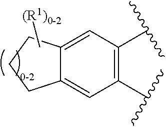

is

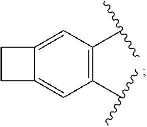 [SMILES: *c1cc2c(cc1*)CC2]

R² is —OCH; R^{4a} is F; R^{4b} is CF₃; R⁵ is

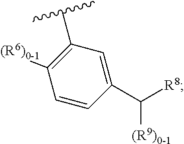

R⁶ is F; R⁸ is —C(═O)OH, —C(═O)NHR^{a}, or CF₃; R⁹ is —NHR^{a}, —NHC(═O)R^{b}, —NHS(═O)ₚC₁₋₄ alkyl or —OC(═O)NHR^{a}; R^{a} is H, C₁₋₃ alkyl, —(CH₂)₀₋₁—C₃₋₆ cycloalkyl, or —(CH₂)₀₋₁-phenyl substituted with 0-2 R^{e}; R^{b} is H or heterocyclyl; R^{e} is C₁₋₃ alkyl, —(CH₂)₀₋₁OR^{f}; and R^{f} is H or C₁₋₃ alkyl.

In another embodiment of Formula (III),

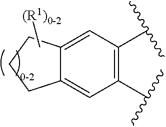

is

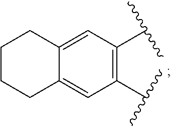 [SMILES: *c1cc2c(cc1*)CCCC2]

R² is —OCH₃; R^{4a} is F; R^{4b} is CF₃; R⁵ is

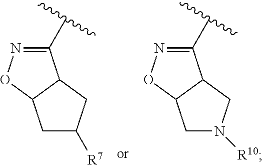

R⁷ is C₁₋₄ alkyl substituted with 0-1 R⁹; R⁹ is —OH, R¹⁰ is —C(═O)R^{b}; R^{b} is H or C₁₋₃ alkyl substituted with 0-4 R^{e}; R^{e} is —(CH₂)₀₋₁OR^{f}; and R^{f} is H or C₁₋₃ alkyl.

In another embodiment of Formula (III),

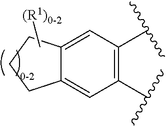

is

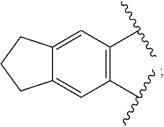 [SMILES: *c1cc2c(cc1*)CCC2]

R² is —OCH₃; R^{4a} is F; R^{4b} is CF₃; R⁵ is

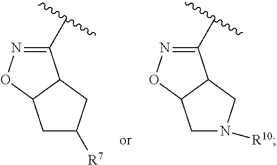

R⁷ is C₁₋₄ alkyl substituted with 0-1 R⁹; R⁹ is —OH, R¹⁰ is —C(═O)R^{b}; R^{b} is H or C₁₋₃ alkyl substituted with 0-4 R^{e}; R^{e} is —(CH₂)₀₋₁OR^{f}; and R^{f} is H or C₁₋₃ alkyl.

In another embodiment of Formula (III),

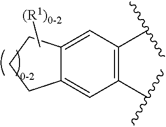

is

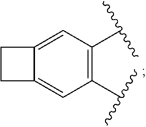 [SMILES: *c1cc2c(cc1*)CC2]

R² is —OCH₃; R^{4a} is F; R^{4b} is CF₃; R⁵ is

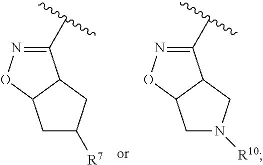

R⁷ is C₁₋₄ alkyl substituted with 0-1 R⁹; R⁹ is —OH, R¹⁰ is —C(═O)R^{b}; R^{b} is H or C₁₋₃ alkyl substituted with 0-4 R^{e}; R^{e} is —(CH₂)₀₋₁OR^{f}; and R^{f} is H or C₁₋₃ alkyl.

Unless specified otherwise, these terms have the following meanings.

“Halo” includes fluoro, chloro, bromo, and iodo.

“Alkyl” or “alkylene” is intended to include both branched and straight-chain saturated aliphatic hydrocarbon groups having the specified number of carbon atoms. For example, “C₁ to C₁₀ alkyl” or “C₁₋₁₀ alkyl” (or alkylene), is intended to include C₁, C₂, C₃, C₄, C₅, C₆, C₇, C₈, C₉, and C₁₀ alkyl groups. Additionally, for example, “C₁ to C₆ alkyl” or “C₁-C₆ alkyl” denotes alkyl having 1 to 6 carbon atoms. Alkyl group can be unsubstituted or substituted with at least one hydrogen being replaced by another chemical group.

Example alkyl groups include, but are not limited to, methyl (Me), ethyl (Et), propyl (e.g., n-propyl and isopropyl), butyl (e.g., n-butyl, isobutyl, t-butyl), and pentyl (e.g., n-pentyl, isopentyl, neopentyl). When “C₀ alkyl” or “C₀ alkylene” is used, it is intended to denote a direct bond. “Alkyl” also includes deuteroalkyl such as CD₃.

“Alkenyl” or “alkenylene” is intended to include hydrocarbon chains of either straight or branched configuration having one or more, preferably one to three, carbon-carbon double bonds that may occur in any stable point along the chain. For example, “C₂ to C₆ alkenyl” or “C₂₋₆ alkenyl” (or alkenylene), is intended to include C₂, C₃, C₄, C₅, and C₆ alkenyl groups; such as ethenyl, propenyl, butenyl, pentenyl, and hexenyl.

“Alkynyl” or “alkynylene” is intended to include hydrocarbon chains of either straight or branched configuration having one or more, preferably one to three, carbon-carbon triple bonds that may occur in any stable point along the chain. For example, “C₂ to C₆ alkynyl” or “C₂₋₆ alkynyl” (or alkynylene), is intended to include C₂, C₃, C₄, C₅, and C₆ alkynyl groups; such as ethynyl, propynyl, butynyl, pentynyl, and hexynyl.

“Carbocycle”, “carbocyclyl”, or “carbocyclic residue” is intended to mean any stable 3-, 4-, 5-, 6-, 7-, or 8-membered monocyclic or bicyclic or 7-, 8-, 9-, 10-, 11-, 12-, or 13-membered bicyclic or tricyclic hydrocarbon ring, any of which may be saturated, partially unsaturated, unsaturated or aromatic. Examples of such carbocyclyls include, but are not limited to, cyclopropyl, cyclobutyl, cyclobutenyl, cyclopentyl, cyclopentenyl, cyclohexyl, cycloheptenyl, cycloheptyl, cycloheptenyl, adamantyl, cyclooctyl, cyclooctenyl, cyclooctadienyl, [3.3.0]bicyclooctane, [4.3.0]bicyclononane, [4.4.0]bicyclodecane (decalin), [2.2.2]bicyclooctane, fluorenyl, phenyl, naphthyl, indanyl, adamantyl, anthracenyl, and tetrahydronaphthyl (tetralin). As shown above, bridged rings are also included in the definition of carbocyclyl (e.g., [2.2.2]bicyclooctane). A bridged ring occurs when one or more carbon atoms link two non-adjacent carbon atoms. Preferred bridges are one or two carbon atoms. It is noted that a bridge always converts a monocyclic ring into a tricyclic ring. When a ring is bridged, the substituents recited for the ring may also be present on the bridge. When the term “carbocyclyl” is used, it is intended to include “aryl,” “cycloalkyl,” and “spirocycloalkyl.” Preferred carbocyclyls, unless otherwise specified, are cyclopropyl, cyclobutyl, cyclopentyl, cyclohexyl, phenyl, and indanyl.

“Cycloalkyl” is intended to mean cyclized alkyl groups, including mono-, bi- or multicyclic ring systems. “C₁ to C₇ cycloalkyl” or “C₃₋₇ cycloalkyl” is intended to include C₃, C₄, C₅, C₆, and C₇ cycloalkyl groups. Non-limiting examples of monocyclic cycloalkyls include cyclopropyl, cyclobutyl, cyclopentyl, cyclohexyl, cycloheptyl and cyclooctyl. Non-limiting examples of multicyclic cycloalkyls include 1-decalinyl, norbornyl and adamantyl.

“Spirocycloalkyl” is intended to mean hydrocarbon bicyclic ring systems with both rings connected through a single atom. The ring can be different in size and nature, or identical in size and nature. Examples include spiropentane, spirohexane, spiroheptane, spirooctane, spirononane, or spirodecane.

“Bicyclic carbocyclyl” or “bicyclic carbocyclic group” is intended to mean a stable 9- or 10-membered carbocyclic ring system that contains two fused rings and consists of carbon atoms. Of the two fused rings, one ring is a benzo ring fused to a second ring; and the second ring is a 5- or 6-membered carbon ring which is saturated, partially unsaturated, or unsaturated. The bicyclic carbocyclic group may be attached to its pendant group at any carbon atom which results in a stable structure. The bicyclic carbocyclic group described herein may be substituted on any carbon if the resulting compound is stable. Examples of a bicyclic carbocyclic group are, but not limited to, naphthyl, 1,2-dihydronaphthyl, 1,2,3,4-tetrahydronaphthyl, and indanyl.

“Aryl” groups refer to monocyclic or polycyclic aromatic hydrocarbons, including, for example, phenyl, naphthyl, and phenanthranyl. Aryl moieties are well known and described, for example, in Lewis, R. J., ed., Hawley's Condensed Chemical Dictionary, 13th Edition, John Wiley & Sons, Inc., New York (1997).

“Benzyl” is intended to mean a methyl group on which one of the hydrogen atoms is replaced by a phenyl group, wherein said phenyl group may optionally be substituted with 1 to 5 groups, preferably 1 to 3 groups.

“Heterocycle”, “heterocyclyl” or “heterocyclic ring” is intended to mean a stable 3-, 4-, 5-, 6-, or 7-membered monocyclic or bicyclic or 7-, 8-, 9-, 10-, 11-, 12-, 13-, or 14-membered polycyclic heterocyclic ring that is saturated, partially unsaturated, or fully unsaturated, and that contains carbon atoms and 1, 2, 3 or 4 heteroatoms independently selected from the group consisting of N, O and S; and including any polycyclic group in which any of the above-defined heterocyclic rings is fused to a benzene ring. The nitrogen and sulfur heteroatoms may optionally be oxidized (i.e., N→O and S(O)ₚ, wherein p is 0, 1 or 2). The nitrogen atom may be substituted or unsubstituted (i.e., N or NR wherein R is H or another substituent, if defined). The heterocyclic ring may be attached to its pendant group at any heteroatom or carbon atom that results in a stable structure. The heterocyclic rings described herein may be substituted on carbon or on a nitrogen atom if the resulting compound is stable. A nitrogen in the heterocyclyl may optionally be quaternized. It is preferred that when the total number of S and O atoms in the heterocyclyl exceeds 1, then these heteroatoms are not adjacent to one another. It is preferred that the total number of S and O atoms in the heterocyclyl is not more than 1. Bridged rings are also included in the definition of heterocyclyl. When the term “heterocyclyl” is used, it is intended to include heteroaryl.

Examples of heterocyclyls include, but are not limited to, acridinyl, azetidinyl, azocinyl, benzimidazolyl, benzofuranyl, benzothiofuranyl, benzothiophenyl, benzoxazolyl, benzoxazolinyl, benzthiazolyl, benztriazolyl, benztetrazolyl, benzisoxazolyl, benzisothiazolyl, benzimidazolinyl, carbazolyl, 4aH-carbazolyl, carbolinyl, chromanyl, chromenyl, cinnolinyl, decahydroquinolinyl, 2H,6H-1,5,2-dithiazinyl, dihydrofuro[2,3-b]tetrahydrofuran, furanyl, furazanyl, imidazolidinyl, imidazolinyl, imidazolyl, 1H-indazolyl, imidazolopyridinyl, indolenyl, indolinyl, indolizinyl, indolyl, 3H-indolyl, isatinoyl, isobenzofuranyl, isochromanyl, isoindazolyl, isoindolinyl, isoindolyl, isoquinolinyl, isothiazolyl, isothiazolopyridinyl, isoxazolyl, isoxazolopyridinyl, methylenedioxyphenyl, morpholinyl, naphthyridinyl, octahydroisoquinolinyl, oxadiazolyl, 1,2,3-oxadiazolyl, 1,2,4-oxadiazolyl, 1,2,5-oxadiazolyl, 1,3,4-oxadiazolyl, oxazolidinyl, oxazolyl, oxazolopyridinyl, oxazolidinylperimidinyl, oxindolyl, pyrimidinyl, phenanthridinyl, phenanthrolinyl, phenazinyl, phenothiazinyl, phenoxathiinyl, phenoxazinyl, phthalazinyl, piperazinyl, piperidinyl, piperidonyl, 4-piperidonyl, piperonyl, pteridinyl, purinyl, pyranyl, pyrazinyl, pyrazolidinyl, pyrazolinyl, pyrazolopyridinyl, pyrazolyl, pyridazinyl, pyridooxazolyl, pyridoimidazolyl, pyridothiazolyl, pyridinyl, pyrimidinyl, pyrrolidinyl, pyrrolinyl, 2-pyrrolidonyl, 2H-pyrrolyl, pyrrolyl, quinazolinyl, quinolinyl, 4H-quinolizinyl, quinoxalinyl, quinuclidinyl, tetrazolyl, tetrahydrofuranyl, tetrahydroisoquinolinyl, tetrahydroquinolinyl, 6H-1,2,5-thiadiazinyl, 1,2,3-thiadiazolyl, 1,2,4-thiadiazolyl, 1,2,5-thiadiazolyl, 1,3,4-thiadiazolyl, thianthrenyl, thiazolyl, thienyl, thiazolopyridinyl, thienothiazolyl, thienooxazolyl, thienoimidazolyl, thiophenyl, triazinyl, 1,2,3-triazolyl, 1,2,4-triazolyl, 1,2,5-triazolyl, 1,3,4-triazolyl, and xanthenyl. Also included are fused ring and spiro compounds containing, for example, the above heterocyclyls.

“Bicyclic heterocyclyl” “bicyclic heterocyclyl” or “bicyclic heterocyclic group” is intended to mean a stable 9- or 10-membered heterocyclic ring system which contains two fused rings and consists of carbon atoms and 1, 2, 3, or 4 heteroatoms independently selected from the group consisting of N, O and S. Of the two fused rings, one ring is a 5- or 6-membered monocyclic aromatic ring comprising a 5-membered heteroaryl ring, a 6-membered heteroaryl ring or a benzo ring, each fused to a second ring. The second ring is a 5- or 6-membered monocyclic ring which is saturated, partially unsaturated, or unsaturated, and comprises a 5-membered heterocyclyl, a 6-membered heterocyclyl or a carbocyclyl (provided the first ring is not benzo when the second ring is a carbocyclyl).

The bicyclic heterocyclic group may be attached to its pendant group at any heteroatom or carbon atom which results in a stable structure. The bicyclic heterocyclic group described herein may be substituted on carbon or on a nitrogen atom if the resulting compound is stable. It is preferred that when the total number of S and O atoms in the heterocyclyl exceeds 1, then these heteroatoms are not adjacent to one another. It is preferred that the total number of S and O atoms in the heterocyclyl is not more than 1.

Examples of a bicyclic heterocyclic group are, but not limited to, quinolinyl, isoquinolinyl, phthalazinyl, quinazolinyl, indolyl, isoindolyl, indolinyl, 1H-indazolyl, benzimidazolyl, 1,2,3,4-tetrahydroquinolinyl, 1,2,3,4-tetrahydroisoquinolinyl, 5,6,7,8-tetrahydroquinolinyl, 2,3-dihydrobenzofuranyl, chromanyl, 1,2,3,4-tetrahydroquinoxalinyl, and 1,2,3,4-tetrahydroquinazolinyl.

“Heteroaryl” is intended to mean stable monocyclic and polycyclic aromatic hydrocarbons that include at least one heteroatom ring member such as sulfur, oxygen, or nitrogen, Heteroaryl groups include, without limitation, pyridyl, pyrimidinyl, pyrazinyl, pyridazinyl, triazinyl, furyl, quinolyl, isoquinolyl, thienyl, imidazolyl, thiazolyl, indolyl, pyrroyl, oxazolyl, benzofuryl, benzothienyl, benzthiazolyl, isoxazolyl, pyrazolyl, triazolyl, tetrazolyl, indazolyl, 1,2,4-thiadiazolyl, isothiazolyl, purinyl, carbazolyl, benzimidazolyl, indolinyl, benzodioxolanyl, and benzodioxane. Heteroaryl groups are substituted or unsubstituted. The nitrogen atom is substituted or unsubstituted (i.e., N or NR wherein R is H or another substituent, if defined). The nitrogen and sulfur heteroatoms may optionally be oxidized (i.e., N→O and S(O)ₚ, wherein p is 0, 1 or 2).

As referred to herein, the term “substituted” means that at least one hydrogen atom is replaced with a non-hydrogen group, provided that normal valencies are maintained and that the substitution results in a stable compound. When a substituent is keto (i.e., ═O), then 2 hydrogens on the atom are replaced. Keto substituents are not present on aromatic moieties. When a ring system (e.g., carbocyclic or heterocyclic) is said to be substituted with a carbonyl group or a double bond, it is intended that the carbonyl group or double bond be part (i.e., within) of the ring. Ring double bonds, as used herein, are double bonds that are formed between two adjacent ring atoms (e.g., C═C, C═N, or N═N).

In cases wherein there are nitrogen atoms (e.g., amines) on compounds of the present invention, these may be converted to N-oxides by treatment with an oxidizing agent (e.g., mCPBA and/or hydrogen peroxides) to afford other compounds of this invention. Thus, shown and claimed nitrogen atoms are considered to cover both the shown nitrogen and its N-oxide (N→O) derivative.

When any variable occurs more than one time in any constituent or formula for a compound, its definition at each occurrence is independent of its definition at every other occurrence. Thus, for example, if a group is shown to be substituted with 0-3 R groups, then said group may optionally be substituted with up to three R groups, and at each occurrence R is selected independently from the definition of R. Also, combinations of substituents and/or variables are permissible only if such combinations result in stable compounds.

When a bond to a substituent is shown to cross a bond connecting two atoms in a ring, then such substituent may be bonded to any atom on the ring. When a substituent is listed without indicating the atom in which such substituent is bonded to the rest of the compound of a given formula, then such substituent may be bonded via any atom in such substituent. Combinations of substituents and/or variables are permissible only if such combinations result in stable compounds.

The invention includes all pharmaceutically acceptable salt forms of the compounds. Pharmaceutically acceptable salts are those in which the counter ions do not contribute significantly to the physiological activity or toxicity of the compounds and as such function as pharmacological equivalents. These salts can be made according to common organic techniques employing commercially available reagents. Some anionic salt forms include acetate, acistrate, besylate, bromide, chloride, citrate, fumarate, glucouronate, hydrobromide, hydrochloride, hydroiodide, iodide, lactate, maleate, mesylate, nitrate, pamoate, phosphate, succinate, sulfate, tartrate, tosylate, and xinofoate. Some cationic salt forms include ammonium, aluminum, benzathine, bismuth, calcium, choline, diethylamine, diethanolamine, lithium, magnesium, meglumine, 4-phenylcyclohexylamine, piperazine, potassium, sodium, tromethamine, and zinc.

Throughout the specification and the appended claims, a given chemical formula or name shall encompass all stereo and optical isomers and racemates thereof where such isomers exist. Unless otherwise indicated, all chiral (enantiomeric and diastereomeric) and racemic forms are within the scope of the invention. Enantiomers and diastereomers are examples of stereoisomers. The term “enantiomer” refers to one of a pair of molecular species that are mirror images of each other and are not superimposable. The term “diastereomer” refers to stereoisomers that are not mirror images. The term “racemate” or “racemic mixture” refers to a composition composed of equimolar quantities of two enantiomeric species, wherein the composition is devoid of optical activity.

The invention includes all tautomeric forms of the compounds, atropisomers and rotational isomers.

All processes used to prepare compounds of the present invention and intermediates made therein are considered to be part of the present invention.

The symbols “R” and “S” represent the configuration of substituents around a chiral carbon atom(s). The isomeric descriptors “R” and “S” are used as described herein for indicating atom configuration(s) relative to a core molecule and are intended to be used as defined in the literature (IUPAC Recommendations 1996, Pure and Applied Chemistry, 68:2193-2222 (1996)).

The term “chiral” refers to the structural characteristic of a molecule that makes it impossible to superimpose it on its mirror image. The term “homochiral” refers to a state of enantiomeric purity. The term “optical activity” refers to the degree to which a homochiral molecule or nonracemic mixture of chiral molecules rotates a plane of polarized light.

The invention is intended to include all isotopes of atoms occurring in the compounds. Isotopes include those atoms having the same atomic number but different mass numbers. By way of general example and without limitation, isotopes of hydrogen include deuterium and tritium. Isotopes of carbon include ¹³C and ¹⁴C. Isotopically-labeled compounds of the invention can generally be prepared by conventional techniques known to those skilled in the art or by processes analogous to those described herein, using an appropriate isotopically-labeled reagent in place of the non-labeled reagent otherwise employed. Such compounds may have a variety of potential uses, for example as standards and reagents in determining biological activity. In the case of stable isotopes, such compounds may have the potential to favorably modify biological, pharmacological, or pharmacokinetic properties.

Throughout the specification and the appended claims, a given chemical formula or name shall encompass all stereo and optical isomers and racemates thereof where such isomers exist. Unless otherwise indicated, all chiral (enantiomeric and diastereomeric) and racemic forms are within the scope of the invention. Many geometric isomers of C═C double bonds, C═N double bonds, ring systems, and the like can also be present in the compounds, and all such stable isomers are contemplated in the present invention. Cis- and trans-(or E- and Z-) geometric isomers of the compounds of the present invention are described and may be isolated as a mixture of isomers or as separated isomeric forms. The present compounds can be isolated in optically active or racemic forms. Optically active forms may be prepared by resolution of racemic forms or by synthesis from optically active starting materials. All processes used to prepare compounds of the present invention and intermediates made therein are considered to be part of the present invention. When enantiomeric or diastereomeric products are prepared, they may be separated by conventional methods, for example, by chromatography or fractional crystallization. Depending on the process conditions the end products of the present invention are obtained either in free (neutral) or salt form. Both the free form and the salts of these end products are within the scope of the invention. If so desired, one form of a compound may be converted into another form. A free base or acid may be converted into a salt; a salt may be converted into the free compound or another salt; a mixture of isomeric compounds of the present invention may be separated into the individual isomers. Compounds of the present invention, free form and salts thereof, may exist in multiple tautomeric forms, in which hydrogen atoms are transposed to other parts of the molecules and the chemical bonds between the atoms of the molecules are consequently rearranged. It should be understood that all tautomeric forms, insofar as they may exist, are included within the invention.

The term “stereoisomer” refers to isomers of identical constitution that differ in the arrangement of their atoms in space. Enantiomers and diastereomers are examples of stereoisomers. The term “enantiomer” refers to one of a pair of molecular species that are mirror images of each other and are not superimposable. The term “diastereomer” refers to stereoisomers that are not mirror images. The term “racemate” or “racemic mixture” refers to a composition composed of equimolar quantities of two enantiomeric species, wherein the composition is devoid of optical activity.

### Biological Methods

RXFP1 Cyclic Adenosine Monophosphate (cAMP) Assays. Human embryonic kidney cells 293 (HEK293) cells and HEK293 cells stably expressing human RXFP1, were cultured in MEM medium supplemented with 10% qualified FBS, and 300 μg/ml hygromycin (Life Technologies). Cells were dissociated and suspended in assay buffer. The assay buffer was HBSS buffer (with calcium and magnesium) containing 20 mM HEPES, 0.05% BSA, and 0.5 mM IBMX. Cells (3000 cells per well, except 1500 cell per well for HEK293 cells stably expressing human RXFP1) were added to 384-well Proxiplates (Perkin-Elmer). Cells were immediately treated with test compounds in DMSO (2% final) at final concentrations in the range of 0.010 nM to 50 μM. Cells were incubated for 30 min at room temperature. The level of intracellular cAMP was determined using the HTRF HiRange cAMP assay reagent kit (Cisbio) according to manufacturer's instructions. Solutions of cryptate conjugated anti-cAMP and d2 fluorophore-labelled cAMP were made in a supplied lysis buffer separately. Upon completion of the reaction, the cells were lysed with equal volume of the d2-cAMP solution and anti-cAMP solution. After a 1 h room temperature incubation, time-resolved fluorescence intensity was measured using the Envision (Perkin-Elmer) at 400 nm excitation and dual emission at 590 nm and 665 nm. A calibration curve was constructed with an external cAMP standard at concentrations ranging from 2.7 μM to 0.1 μM by plotting the fluorescent intensity ratio from 665 nm emission to the intensity from the 590 nm emission against cAMP concentrations. The potency and activity of a compound to inhibit cAMP production was then determined by fitting to a 4-parametric logistic equation from a plot of cAMP level versus compound concentrations.

The examples disclosed below were tested in the human RXFP1 (hRXFP1) HEK293 cAMP assay described above and found to have agonist activity. Tables 1-3, lists EC₅₀ values in the hRXFP1 HEK293 cAMP assay measured for the examples.

Table 1 lists EC₅₀ values in the hRXFP1 HEK293 cAMP assay measured for the phenylcyclohexyl examples.

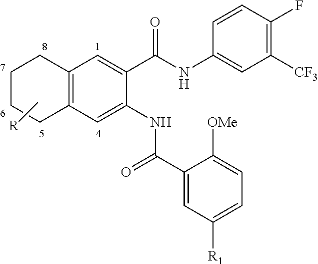

TABLE 2

| | Coupling | | | CAMP hRXFP1 |
|---|---|---|---|---|
| | Inter- | | | HEK293 Assay |
| Example # | mediates | R | R1 | EC₅₀ (nM) |
| 1 | 7-6/1-1 | H | 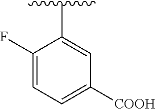 [SMILES: *c1cc(C(=O)O)ccc1F] | 390 |
| 2 | 13-2/ tetrahydro-2H- pyran-4- carbonyl chloride | H | 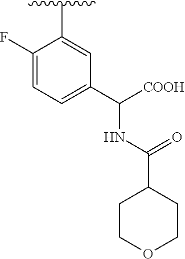 [SMILES: *c1cc(C(NC(=O)C2CCOCC2)C(=O)O)ccc1F] | 1.8 |
| 3 | 13-2/TFA deprotection | H | 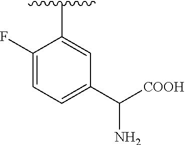 [SMILES: *c1cc(C(N)C(=O)O)ccc1F] | 2,300 |
| 4 | 14-1/15-1 | H | 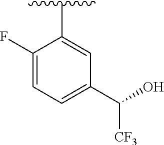 [SMILES: *c1cc([C@H](O)C(F)(F)F)ccc1F] | 860 |
| 5 | Example 4/phenyl isocyanate | H | 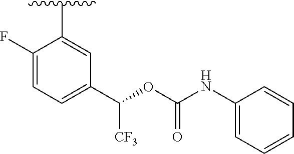 [SMILES: *c1cc([C@H](OC(=O)Nc2ccccc2)C(F)(F)F)ccc1F] | 33 |
| 6 | 4/cyclobutylamine | H | 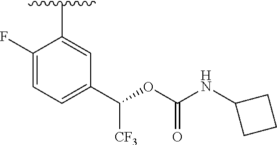 [SMILES: *c1cc([C@H](OC(=O)NC2CCC2)C(F)(F)F)ccc1F] | 15 |
| 7 | Example 4/3,3- difluorocyclobutan- 1-amine | H | 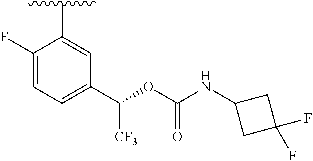 [SMILES: *c1cc([C@H](OC(=O)NC2CC(F)(F)C2)C(F)(F)F)ccc1F] | 62 |
| 8 | 15-1/16-1 | H | 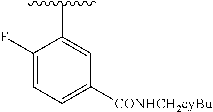 | 4,200 |
| 9 | 15-1/23-1 | H | 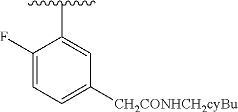 | 22 |
| 12 | 7-6/6-2 | H | 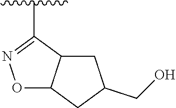 [SMILES: *C1=NOC2CC(CO)CC12] | 290 |
| 13 | 7-6/6-8 | H | 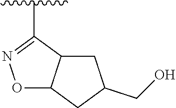 [SMILES: *C1=NOC2CC(CO)CC12] | 19 |
| 14 | 7-6/6-10 | H | 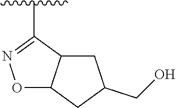 [SMILES: *C1=NOC2CC(CO)CC12] | 82 |
| 15 | 7-6/12-2 | H | 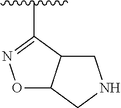 [SMILES: *C1=NOC2CNCC12] | 3,700 |
| 16 | 7-6/19-2 | H | 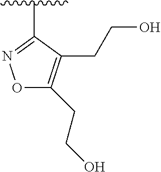 [SMILES: *c1noc(CCO)c1CCO] | 5,000 |
| 17 | 8-5/20-2 | 5- CO | 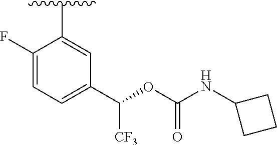 [SMILES: *c1cc([C@H](OC(=O)NC2CCC2)C(F)(F)F)ccc1F] | 22 |
| 18 | 8-5/1-1 | 5- CO | 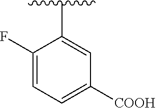 [SMILES: *c1cc(C(=O)O)ccc1F] | 1,100 |
| 19 | 15-1/21-2 | H | 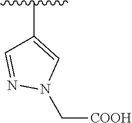 [SMILES: *c1cnn(CC(=O)O)c1] | 1,300 |
| 20 | 7-6/22-2 | H | 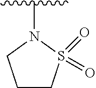 [SMILES: *N1CCCS1(=O)=O] | 2,200 |

lists EC50 values in the hRXFP1 HEK293 cAMP assay measured for the phenylcyclobutane examples.

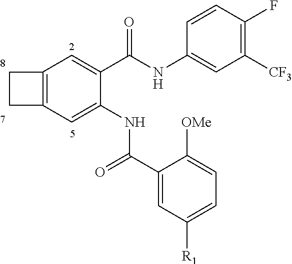

TABLE 3

| | Coupling | | cAMP hRXFP1 HEK293 |
|---|---|---|---|
| Example | Inter- | | Assay |
| # | mediates | R1 | EC₅₀ (nM) |
| 21 | 9-4/1-1 | 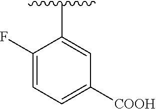 [SMILES: *c1cc(C(=O)O)ccc1F] | 2,400 |
| 22 | 9-4/3-6 | 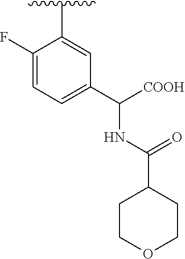 [SMILES: *c1cc(C(NC(=O)C2CCOCC2)C(=O)O)ccc1F] | 8.0 |
| 23 | 9-4/2-6 | 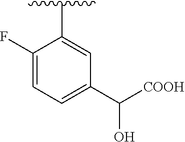 [SMILES: *c1cc(C(O)C(=O)O)ccc1F] | 660 |

lists EC50 values in the hRXFPI HEK293 cAMP assay measured for the Phenylcyclopentyl examples.

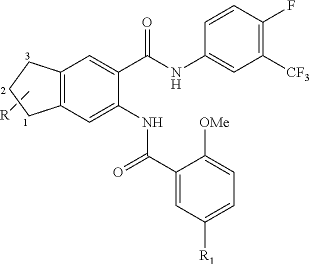

| | | | | cAMP hRXFP1 |
|---|---|---|---|---|
| Example | Coupling | | | HEK293 Assay |
| # | Intermediates | R | R/R1 | EC₅₀ (nM) |
| 24 | 10-4/1-1 | H | 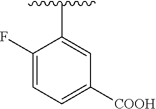 [SMILES: *c1cc(C(=O)O)ccc1F] | 3437 |
| 25 | 10-4/3-6 | H | 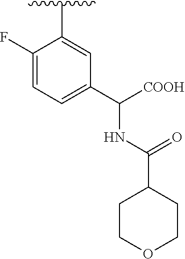 [SMILES: *c1cc(C(NC(=O)C2CCOCC2)C(=O)O)ccc1F] | 11 |
| 26 | 11-3/1-1 | 1-CO | 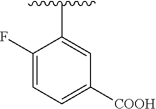 [SMILES: *c1cc(C(=O)O)ccc1F] | 230 |
| 27 | 11-3/ | 1-CO | 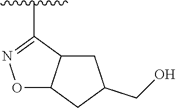 [SMILES: *C1=NOC2CC(CO)CC12] | 34 |
| 28 | 11-3/ | 1-CO | 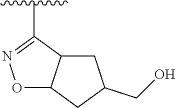 [SMILES: *C1=NOC2CC(CO)CC12] | 160 |
| 29 | Example 26/1-1 | 1- COH | 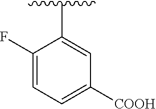 [SMILES: *c1cc(C(=O)O)ccc1F] | 2700 |

### Pharmaceutical Compositions and Methods of Use

The compounds of Formula (I) are RXFP1 receptor agonists and may find use in the treatment of medical indications such as heart failure, fibrotic diseases, and related diseases such as lung disease (e.g., idiopathic pulmonary fibrosis), kidney disease (e.g., chronic kidney disease), or hepatic disease (e.g., non-alcoholic steatohepatitis and portal hypertension).

Another aspect of the invention is a pharmaceutical composition comprising a compound of Formula (I) and a pharmaceutically acceptable carrier.

Another aspect of the invention is a pharmaceutical composition comprising a compound of Formula (I) for the treatment of a relaxin-associated disorder and a pharmaceutically acceptable carrier.

Another aspect of the invention is a method of treating a disease associated with relaxin comprising administering an effective amount of a compound of Formula (I).

Another aspect of the invention is a method of treating a cardiovascular disease comprising administering an effective amount of a compound of Formula (I) to a patient in need thereof.

Another aspect of the invention is a method of treating heart failure comprising administering an effective amount of a compound of Formula (I) to a patient in need thereof.

Another aspect of the invention is a method of treating fibrosis comprising administering a therapeutically effective amount of a compound of Formula (I) to a patient in need thereof.

Another aspect of the invention is a method of treating a disease associated with fibrosis comprising administering a therapeutically effective amount of a compound of Formula (I) to a patient in need thereof.

Another aspect of the invention is a method of treating or preventing kidney failure, comprising administering a therapeutically effective amount of a compound of Formula (I) to a patient in need thereof.

Another aspect of the invention is a method of improving, stabilizing or restoring renal function in a patient in need thereof, comprising administering a therapeutically effective amount of a compound of Formula (I) to the patient.

Unless otherwise specified, the following terms have the stated meanings.

The term “patient” or “subject” refers to any human or non-human organism that could potentially benefit from treatment with a RXFP1 agonist as understood by practioners in this field. Exemplary subjects include human beings of any age with risk factors for cardiovascular disease. Common risk factors include, but are not limited to, age, sex, weight, family history, sleep apnea, alcohol or tobacco use, physical inactivity, arrhythmia, or signs of insulin resistance such as acanthosis nigricans, hypertension, dyslipidemia, or polycystic ovary syndrome (PCOS).

“Treating” or “treatment” cover the treatment of a disease-state as understood by practitioners in this field and include the following: (a) inhibiting the disease-state, i.e., arresting it development; (b) relieving the disease-state, i.e., causing regression of the disease state; and/or (c) preventing the disease-state from occurring in a mammal, in particular, when such mammal is predisposed to the disease-state but has not yet been diagnosed as having it.

“Preventing” or “prevention” cover the preventive treatment (i.e., prophylaxis and/or risk reduction) of a subclinical disease-state aimed at reducing the probability of the occurrence of a clinical disease-state as understood by practitioners in this field. Patients are selected for preventative therapy based on factors that are known to increase risk of suffering a clinical disease state compared to the general population. “Prophylaxis” therapies can be divided into (a) primary prevention and (b) secondary prevention. Primary prevention is defined as treatment in a subject that has not yet presented with a clinical disease state, whereas secondary prevention is defined as preventing a second occurrence of the same or similar clinical disease state. “Risk reduction” or “reducing risk” covers therapies that lower the incidence of development of a clinical disease state. As such, primary and secondary prevention therapies are examples of risk reduction.

“Therapeutically effective amount” is intended to include an amount of a compound of the present invention that is effective when administered alone or in combination with other agents to treat disorders as understood by practitioners in this field. When applied to a combination, the term refers to combined amounts of the active ingredients that result in the preventive or therapeutic effect, whether administered in combination, serially, or simultaneously.

“Disorders of the cardiovascular system” or “cardiovascular disorders” include for example the following disorders: hypertension (high blood pressure), peripheral and cardiac vascular disorders, coronary heart disease, stable and unstable angina pectoris, heart attack, myocardial insufficiency, abnormal heart rhythms (or arrhythmias), persistent ischemic dysfunction (“hibernating myocardium”), temporary postischemic dysfunction (“stunned myocardium”), heart failure, disturbances of peripheral blood flow, acute coronary syndrome, heart failure, heart muscle disease (cardiomyopathy), myocardial infarction and vascular disease (blood vessel disease).

“Heart failure” includes both acute and chronic manifestations of heart failure, as well as more specific or related types of disease, such as advanced heart failure, post-acute heart failure, cardio-renal syndrome, heart failure with impaired kidney function, chronic heart failure, chronic heart failure with mid-range ejection fraction (HFmEF), compensated heart failure, decompensated heart failure, right heart failure, left heart failure, global failure, ischemic cardiomyopathy, dilated cardiomyopathy, heart failure associated with congenital heart defects, heart valve defects, heart failure associated with heart valve defects, mitral stenosis, mitral insufficiency, aortic stenosis, aortic insufficiency, tricuspid stenosis, tricuspid insufficiency, pulmonary stenosis, pulmonary valve insufficiency, heart failure associated with combined heart valve defects, myocardial inflammation (myocarditis), chronic myocarditis, acute myocarditis, viral myocarditis, diabetic heart failure, alcoholic cardiomyopathy, heart failure associated with cardiac storage disorders, diastolic heart failure, systolic heart failure, acute phases of worsening heart failure, heart failure with preserved ejection fraction (HFpEF), heart failure with reduced ejection fraction (HFrEF), chronic heart failure with reduced ejection fraction (HFrEF), chronic heart failure with preserved ejection fraction (HFpEF), post myocardial remodeling, angina, hypertension, pulmonary hypertension and pulmonary artery hypertension.

“Fibrotic disorders” encompasses diseases and disorders characterized by fibrosis, including among others the following diseases and disorders: hepatic fibrosis, cirrhosis of the liver. NASH, pulmonary fibrosis or lung fibrosis, cardiac fibrosis, endomyocardial fibrosis, nephropathy, glomerulonephritis, interstitial renal fibrosis, fibrotic damage resulting from diabetes, bone marrow fibrosis and similar fibrotic disorders, scleroderma, morphea, keloids, hypertrophic scarring (also following surgical procedures), naevi, diabetic retinopathy, proliferative vitreoretinopathy and disorders of the connective tissue (for example sarcoidosis).

Relaxin-associated disorders include but are not limited to disorders of the cardiovascular system and fibrotic disorders.

The compounds of this invention can be administered by any suitable means, for example, orally, such as tablets, capsules (each of which includes sustained release or timed release formulations), pills, powders, granules, elixirs, tinctures, suspensions (including nanosuspensions, microsuspensions, spray-dried dispersions), syrups, and emulsions; sublingually; bucally; parenterally, such as by subcutaneous, intravenous, intramuscular, or intrasternal injection, or infusion techniques (e.g., as sterile injectable aqueous or non-aqueous solutions or suspensions); nasally, including administration to the nasal membranes, such as by inhalation spray; topically, such as in the form of a cream or ointment; or rectally such as in the form of suppositories. They can be administered alone, but generally will be administered with a pharmaceutical carrier selected on the basis of the chosen route of administration and standard pharmaceutical practice.

“Pharmaceutical composition” means a composition comprising a compound of the invention in combination with at least one additional pharmaceutically acceptable carrier. A “pharmaceutically acceptable carrier” refers to media generally accepted in the art for the delivery of biologically active agents to animals, in particular, mammals, including, i.e., adjuvant, excipient or vehicle, such as diluents, preserving agents, fillers, flow regulating agents, disintegrating agents, wetting agents, emulsifying agents, suspending agents, sweetening agents, flavoring agents, perfuming agents, anti-bacterial agents, anti-fungal agents, lubricating agents and dispensing agents, depending on the nature of the mode of administration and dosage forms.

Pharmaceutically acceptable carriers are formulated according to a number of factors well within the purview of those of ordinary skill in the art. These include, without limitation: the type and nature of the active agent being formulated; the subject to which the agent-containing composition is to be administered; the intended route of administration of the composition; and the therapeutic indication being targeted. Pharmaceutically acceptable carriers include both aqueous and non-aqueous liquid media, as well as a variety of solid and semi-solid dosage forms. Such carriers can include a number of different ingredients and additives in addition to the active agent, such additional ingredients being included in the formulation for a variety of reasons, e.g., stabilization of the active agent, binders, etc., well known to those of ordinary skill in the art. Descriptions of suitable pharmaceutically acceptable carriers, and factors involved in their selection, are found in a variety of readily available sources such as, for example, Allen, L. V., Jr. et al., Remington: The Science and Practice of Pharmacy (2 Volumes), 22nd Edition, Pharmaceutical Press (2012).

The dosage regimen for the compounds of the present invention will, of course, vary depending upon known factors, such as the pharmacodynamic characteristics of the particular agent and its mode and route of administration: the species, age, sex, health, medical condition, and weight of the recipient; the nature and extent of the symptoms; the kind of concurrent treatment; the frequency of treatment; the route of administration, the renal and hepatic function of the patient, and the effect desired.

By way of general guidance, the daily oral dosage of each active ingredient, when used for the indicated effects, will range between about 0.01 to about 5000 mg per day, preferably between about 0.1 to about 1000 mg per day, and most preferably between about 0.1 to about 250 mg per day. Intravenously, the most preferred doses will range from about 0.01 to about 10 mg/kg/minute during a constant rate infusion. Compounds of this invention may be administered in a single daily dose, or the total daily dosage may be administered in divided doses of two, three, or four times daily.

The compounds are typically administered in admixture with suitable pharmaceutical diluents, excipients, or carriers (collectively referred to herein as pharmaceutical carriers) suitably selected with respect to the intended form of administration, e.g., oral tablets, capsules, elixirs, and syrups, and consistent with conventional pharmaceutical practices.

Dosage forms (pharmaceutical compositions) suitable for administration may contain from about 1 milligram to about 2000 milligrams of active ingredient per dosage unit. In these pharmaceutical compositions the active ingredient will ordinarily be present in an amount of about 0.1-95% by weight based on the total weight of the composition. A typical capsule for oral administration contains at least one of the compounds of the present invention (250 mg), lactose (75 mg), and magnesium stearate (15 mg). The mixture is passed through a 60 mesh sieve and packed into a No. 1 gelatin capsule. A typical injectable preparation is produced by aseptically placing at least one of the compounds of the present invention (250 mg) into a vial, aseptically freeze-drying and sealing. For use, the contents of the vial are mixed with 2 mL of physiological saline, to produce an injectable preparation.

The compounds may be employed in combination with other suitable therapeutic agents useful in the treatment of diseases or disorders including: anti-atherosclerotic agents, anti-dyslipidemic agents, anti-diabetic agents, anti-hyperglycemic agents, anti-hyperinsulinemic agents, anti-thrombotic agents, anti-retinopathic agents, anti-neuropathic agents, anti-nephropathic agents, anti-ischemic agents, anti-hypertensive agents, anti-obesity agents, anti-hyperlipidemic agents, anti-hypertriglyceridemic agents, anti-hypercholesterolemic agents, anti-restenotic agents, anti-pancreatic agents, lipid lowering agents, anorectic agents, memory enhancing agents, anti-dementia agents, cognition promoting agents, appetite suppressants, agents for treating heart failure, agents for treating peripheral arterial disease, agents for treating malignant tumors, and anti-inflammatory agents.

The additional therapeutic agents may include ACE inhibitors, β-blockers, diuretics, mineralocorticoid receptor antagonists, ryanodine receptor modulators, SERCA2a activators, renin inhibitors, calcium channel blockers, adenosine A1 receptor agonists, partial adenosine A1 receptor, dopamine P-hydroxylase inhibitors, angiotensin II receptor antagonists, angiotensin II receptor antagonists with biased agonism for select cell signaling pathways, combinations of angiotensin II receptor antagonists and neprilysin enzyme inhibitors, neprilysin enzyme inhibitors, soluble guanylate cyclase activators, myosin ATPase activators, rho-kinase 1 inhibitors, rho-kinase 2 inhibitors, apelin receptor agonists, nitroxyl donating compounds, calcium-dependent kinase II inhibitors, antifibrotic agents, galectin-3 inhibitors, vasopressin receptor antagonists, FPR2 receptor modulators, natriuretic peptide receptor agonists, transient receptor potential vanilloid-4 channel blockers, anti-arrhythmic agents, I_{f} “funny current” channel blockers, nitrates, digitalis compounds, inotropic agents and β-receptor agonists, cell membrane resealing agents for example Poloxamer 188, anti-hyperlipidemic agents, plasma HDL-raising agents, anti-hypercholesterolemic agents, cholesterol biosynthesis inhibitors (such as HMG CoA reductase inhibitors), LXR agonist, FXR agonist, probucol, raloxifene, nicotinic acid, niacinamide, cholesterol absorption inhibitors, bile acid sequestrants, anion exchange resins, quaternary amines, cholestyramine, colestipol, low density lipoprotein receptor inducers, clofibrate, fenofibrate, bezafibrate, ciprofibrate, gemfibrizol, vitamin B6, vitamin B12, anti-oxidant vitamins, anti-diabetes agents, platelet aggregation inhibitors, fibrinogen receptor antagonists, aspirin and fibric acid derivatives, PCSK9 inhibitors, aspirin, and P2Y12 Inhibitors such as Clopidogrel.

The additional therapeutic agents may also include nintedanib, Pirfenidone, LPA1 antagonists, LPA1 receptor antagonists, GLP1 analogs, tralokinumab (IL-13, AstraZeneca), vismodegib (hedgehog antagonist, Roche), PRM-151 (pentraxin-2, TGF beta-1, Promedior), SAR-156597 (bispecific Mab IL-4&IL-13, Sanofi), simtuzumab ((anti-lysyl oxidase-like 2 (anti-LOXL2) antibody, Gilead), CKD-942, PTL-202 (PDE inh./pentoxify.lline/NAC oral control, release, Pacific Ther.), omipalisib (oral PI3K/mTOR inhibitor, GSK), IW-001 (oral sol. bovine type V collagen mod., ImmuneWorks), STX-100 (integrin alpha V/beta-6 ant, Stromedix/Biogen), Actimmune (IFN gamma), PC-SOD (midismase; inhaled, LTT Bio-Pharma/CKD Pharm), lebrikizumab (anti-IL-13 SC humanized mAb, Roche), AQX-1125 (SHIP1 activator, Aquinox), CC-539 (JNK inhibitor, Celgene), FG-3019 (FibroGen), SAR-100842 (Sanofi), and obeticholic acid (OCA or INT-747, Intercept).

The above other therapeutic agents, when employed in combination with the compounds of the present invention may be used, for example, in those amounts indicated in the Physicians' Desk Reference, as in the patents set out above, or as otherwise determined by practitioners in the art.

Particularly when provided as a single dosage unit, the potential exists for a chemical interaction between the combined active ingredients. For this reason, when the compound of the present invention and a second therapeutic agent are combined in a single dosage unit they are formulated such that although the active ingredients are combined in a single dosage unit, the physical contact between the active ingredients is minimized (that is, reduced). For example, one active ingredient may be enteric coated. By enteric coating one of the active ingredients, it is possible not only to minimize the contact between the combined active ingredients, but also, it is possible to control the release of one of these components in the gastrointestinal tract such that one of these components is not released in the stomach but rather is released in the intestines. One of the active ingredients may also be coated with a material that affects a sustained-release throughout the gastrointestinal tract and also serves to minimize physical contact between the combined active ingredients. Furthermore, the sustained-released component can be additionally enteric coated such that the release of this component occurs only in the intestine. Still another approach would involve the formulation of a combination product in which the one component is coated with a sustained and/or enteric release polymer, and the other component is also coated with a polymer such as a low viscosity grade of hydroxypropyl methylcellulose (HPMC) or other appropriate materials as known in the art, in order to further separate the active components. The polymer coating serves to form an additional barrier to interaction with the other component.

The compounds of the present invention are also useful as standard or reference compounds, for example as a quality standard or control, in tests or assays involving RXFP1. Such compounds may be provided in a commercial kit, for example, for use in pharmaceutical research involving RXFP1. For example, a compound of the present invention could be used as a reference in an assay to compare its known activity to a compound with an unknown activity. This would ensure the experimenter that the assay was being performed properly and provide a basis for comparison, especially if the test compound was a derivative of the reference compound. When developing new assays or protocols, compounds according to the present invention could be used to test their effectiveness. The compounds of the present invention may also be used in diagnostic assays involving RXFP1.

The present invention also encompasses an article of manufacture. As used herein, article of manufacture is intended to include, but not be limited to, kits and packages. The article of manufacture of the present invention, comprises: (a) a first container; (b) a pharmaceutical composition located within the first container, wherein the composition, comprises a first therapeutic agent, comprising a compound of the present invention or a pharmaceutically acceptable salt form thereof, and, (c) a package insert stating that the pharmaceutical composition can be used for the treatment of dyslipidemias and the sequelae thereof. In another embodiment, the package insert states that the pharmaceutical composition can be used in combination (as defined previously) with a second therapeutic agent for the treatment of dyslipidemias and the sequelae thereof. The article of manufacture can further comprise: (d) a second container, wherein components (a) and (b) are located within the second container and component (c) is located within or outside of the second container. Located within the first and second containers means that the respective container holds the item within its boundaries.

The first container is a receptacle used to hold a pharmaceutical composition. This container can be for manufacturing, storing, shipping, and/or individual/bulk selling. First container is intended to cover a bottle, jar, vial, flask, syringe, tube (e.g., for a cream preparation), or any other container used to manufacture, hold, store, or distribute a pharmaceutical product.

The second container is one used to hold the first container and, optionally, the package insert. Examples of the second container include, but are not limited to, boxes (e.g., cardboard or plastic), crates, cartons, bags (e.g., paper or plastic bags), pouches, and sacks. The package insert can be physically attached to the outside of the first container via tape, glue, staple, or another method of attachment, or it can rest inside the second container without any physical means of attachment to the first container. Alternatively, the package insert is located on the outside of the second container. When located on the outside of the second container, it is preferable that the package insert is physically attached via tape, glue, staple, or another method of attachment. Alternatively, it can be adjacent to or touching the outside of the second container without being physically attached.

The package insert is a label, tag, marker, etc. that recites information relating to the pharmaceutical composition located within the first container. The information recited will usually be determined by the regulatory agency governing the area in which the article of manufacture is to be sold (e.g., the United States Food and Drug Administration). Preferably, the package insert specifically recites the indications for which the pharmaceutical composition has been approved. The package insert may be made of any material on which a person can read information contained therein or thereon. Preferably, the package insert is a printable material (e.g., paper, plastic, cardboard, foil, adhesive-backed paper or plastic, etc.) on which the desired information has been formed (e.g., printed or applied).

### Synthesis Schemes

The compounds of this invention can be made by various methods known in the art including those of the following schemes and in the specific embodiments section. The structure numbering and variable numbering shown in the synthetic schemes are distinct from, and should not be confused with, the structure or variable numbering in the claims or the rest of the specification. The variables in the schemes are meant only to illustrate how to make some of the compounds of this invention.

It will also be recognized that another major consideration in the planning of any synthetic route in this field is the judicious choice of the protecting group used for protection of the reactive functional groups present in the compounds described in this invention. An authoritative account describing the many alternatives to the trained practitioner is Greene, T. W. et al., Protecting Groups in Organic Synthesis, 4th Edition, Wiley (2007)).

Common cyclobutane intermediates of this invention can be accessed via readily available bicyclo[4.2.0]octa-1(6),2,4-triene-3-carboxylic acid. Nitration using KNO₃ in cold sulfuric acid affords the nitro intermediate 1-1 which can be coupled with amines of this invention to afford the amide intermediate I-II. Reduction of the nitro group followed by standard peptide coupling techniques or alternative methods known in the art with acid derivatives of this invention should afford compounds of this invention.

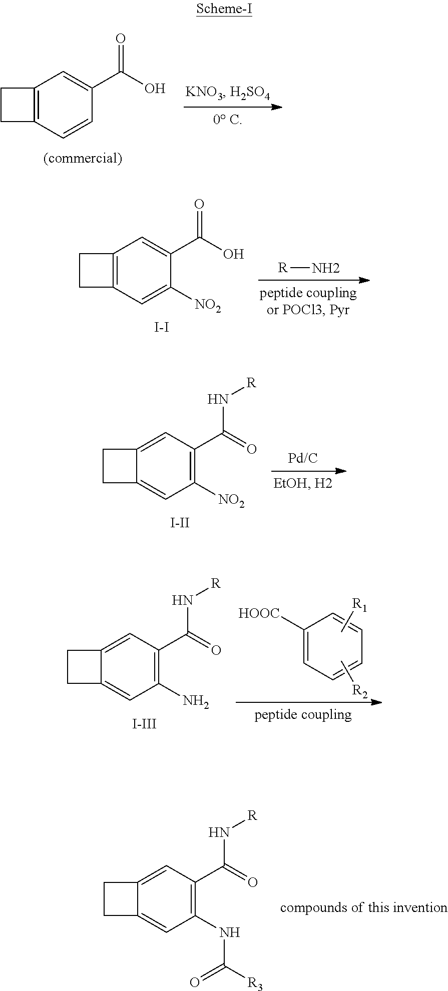

Alternatively, bicyclo[4.2.0]octa-1(6),2,4-triene-3-carboxylic acid (scheme-II) can be brominated via NBS or oxidized with SeO₂ to afford either broom or keto intermediates which were appropriately functionalized to afford substituted phenylcyclobutane intermediates of this invention.

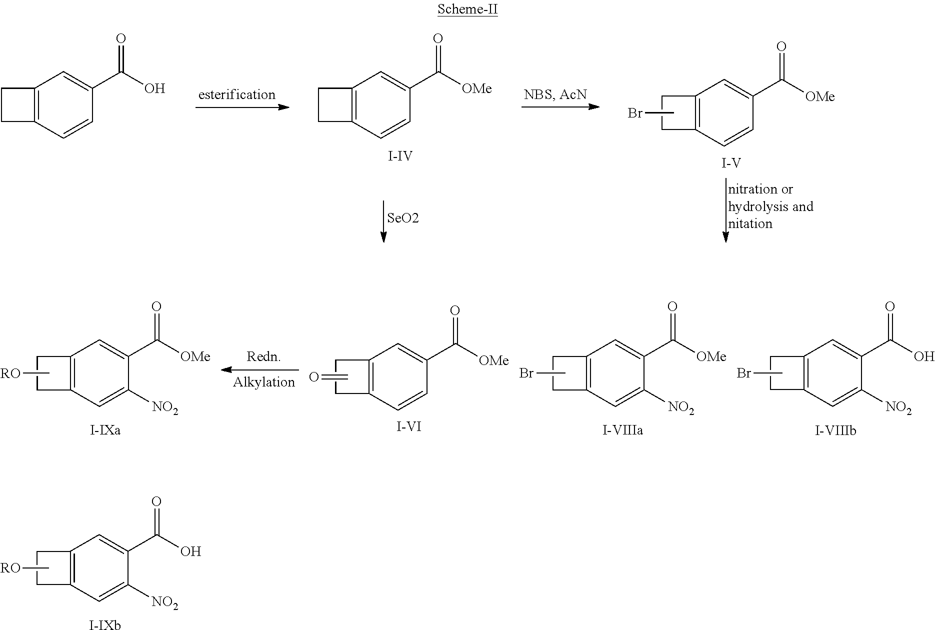

The halogen and the keto intermediates can be converted to other substituted compounds of this invention. The acid and nitro moieties can then be coupled with amines, reduced and re-coupled with acids to afford compounds of this invention.

Alternatively, phenylcyclobutane (scheme-III) can be nitrated (J. Org. Chem., Vol. 44, No. 18, 1979) and bromination with NBS to afford the bromo-aminophenylcyclobutane intermediate I-X. Palladium catalyzed carbonylation of the intermediate can lead to the requisite cyclobutylbenzoate derivative I-XI. This intermediate can then be converted to the compounds of this invention as shown in scheme-I.

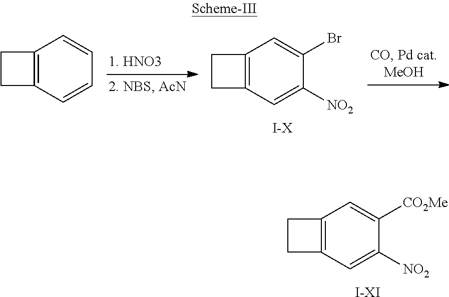

In a similar sequence, phenylcyclohexyl derivatives can be synthesized from readily available 5,6,7,8-tetrahydronaphthalene-2-carboxylic acid as shown in scheme-IV.

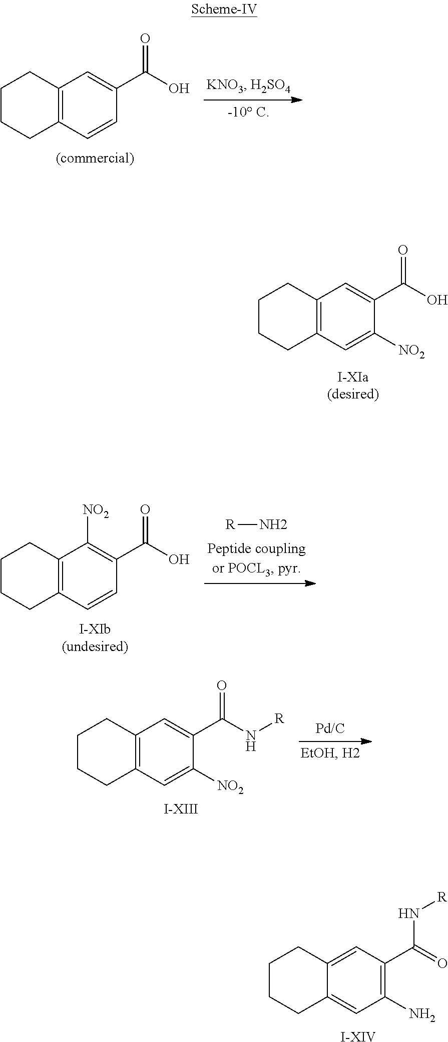

Alternatively, substituted phenylcyclohexyl compounds can be obtained from commercially available 6-bromo-3,4-dihydronaphthalen-1(2H)-one (scheme-V). Nitration followed palladium catalyzed carbonylation afford the desired nitro-ester derivative which can be further functionalized as outlined in Scheme-1 to afford compounds of this invention.

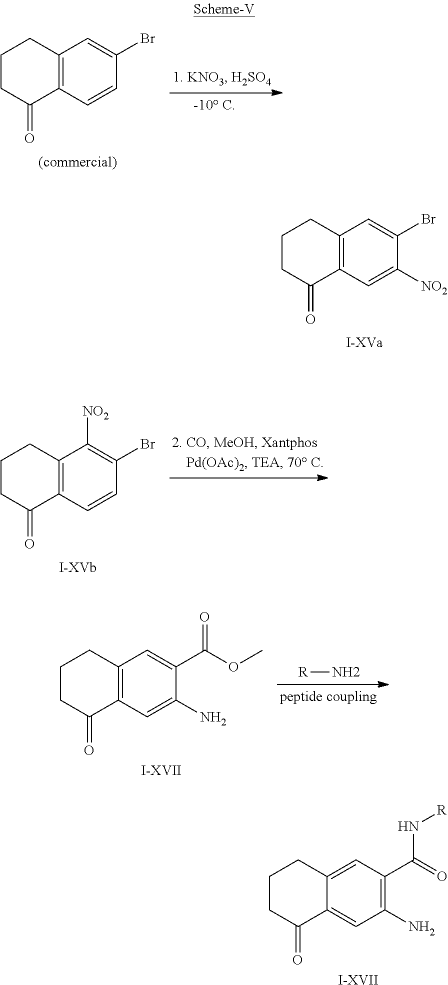

The carbonyl functionality can be reduced with NaBH₄ and subsequently alkylated or displaced with other nucleophiles via Mitsunobu conditions or via a tosylate or can be directly reduced to the methylene compounds of this invention. Alternatively via protection of the amide and aniline groups, the ketone can be alkylated via LDA and a suitable electrophile to afford additional compounds of this invention. The intermediate ketone can be treated with Grignard and zinc reagents to afford additional Examples of this invention.

Phenylcyclopentyl analogs can also be accessed as shown in the general Scheme-VI from commercially available 2,3-dihydro-1H-indene-5-carboxylic acid. Following the sequence of reactions outlined in Scheme-I the phenylcyclopentyl intermediates of this invention can be readily accessed.

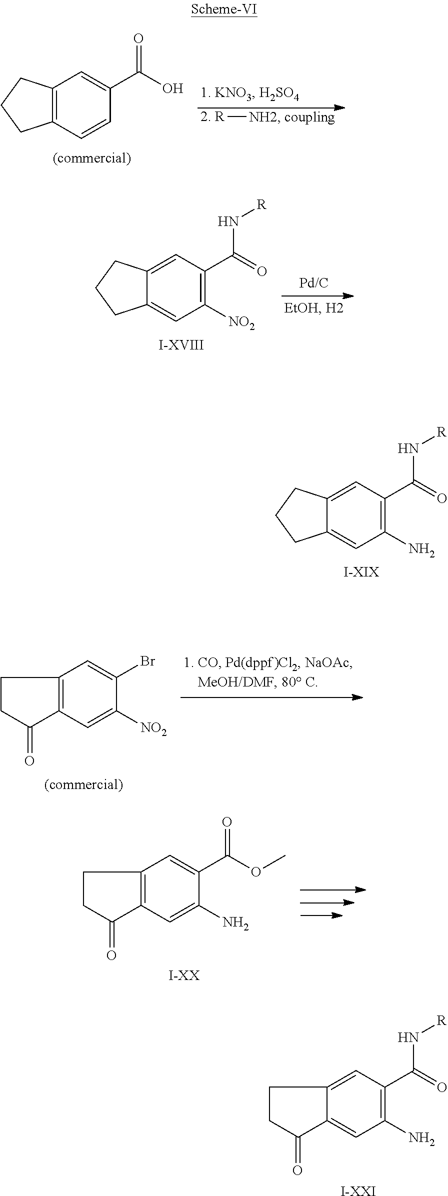

Substituted benzoic acids or heterocyclic acid intermediates of this invention can be synthesized as shown in scheme-VII. In each case, the aryl group can also be substituted with a heteroaryl group and the same sequence of reactions can also afford the corresponding heteroaryl compounds of this invention.

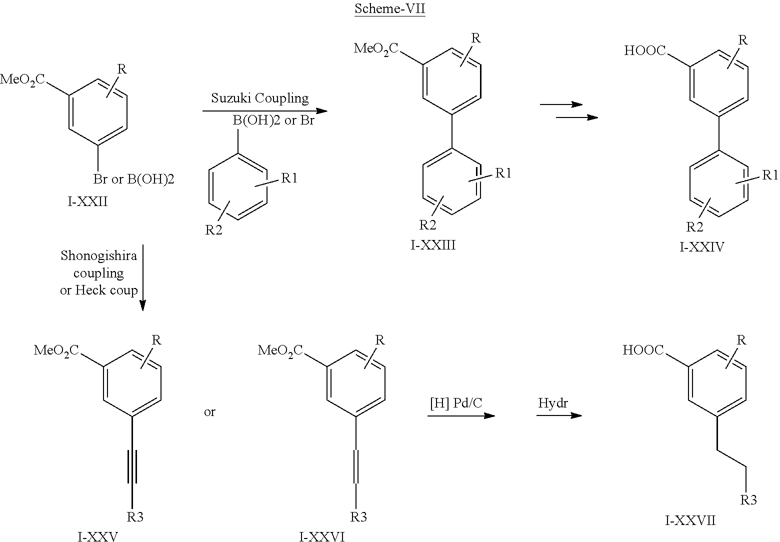

Isoxazoline intermediates of this invention can be obtained from commercially available aryl or heteroaryl carboxaldehydes as shown in scheme-VIII. Conversion of the aldehyde to the oxime, followed by treatment with NCS should provide the requisite phenylchlorooxime or heteroarylchlorooxime. Treatment of the chlorooxime derivatives of this invention with an appropriate olefin should afford isoxazoline intermediates shown in scheme-VIII.

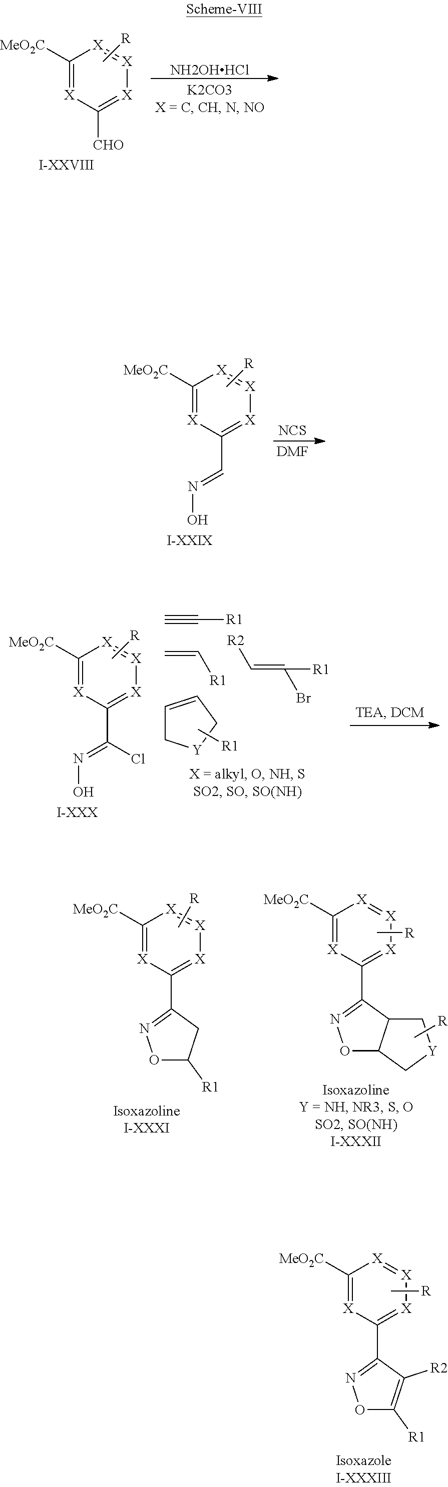

Conversely treatment of the chlorooximes of this invention with an acetylenic or bromoolefine intermediates afforded isoxazole derivatives of this invention. Heteroaryl carboxaldehydes can also be subjected to similar conditions to afford appropriate heteroaryl isoxazoline or isoxazole intermediates of this invention. Mixtures of enantiomers can be separated via chiral SFC. Other intermediates of this invention are described below.

### Chemical Methods and Synthesis

Abbreviations are defined as follows: “1×” for once, “2×” for twice, “3×” for thrice, “° C.” for degrees Celsius, “aq” for aqueous, “eq” or “equiv.” for equivalent or equivalents, “g” for gram or grams, “mg” for milligram or milligrams, “L” for liter or liters, “mL” for milliliter or milliliters, “L” for microliter or microliters, “N” for normal, “M” for molar, “nM” for nanomolar, “pM” for picomolar, “mol” for mole or moles, “mmol” for millimole or millimoles, “min” for minute or minutes, “h” for hour or hours, “rt” for mom temperature, “RT” for retention time, “atm” for atmosphere, “psi” for pounds per square inch, “conc.” for concentrate, “aq” for “aqueous”, “sat.” for saturated, “MW” for molecular weight, “MS” or “Mass Spec” for mass spectrometry, “ESI” for electrospray ionization mass spectroscopy, “LC-MS” for liquid chromatography mass spectrometry. “HPLC” for high pressure liquid chromatography. “RP HPLC” for reverse phase HPLC. “NMR” for nuclear magnetic resonance spectroscopy. “SFC” for super critical fluid chromatography. “1H” for proton. “δ” for delta, “s” for singlet, “d” for doublet, “t” for triplet, “q” for quartet, “in” for multiplet. “br” for broad. “Hz” for hertz. “MHz” for megahertz, and “α” “β”, “R”, “S”, “E”, and “Z” are stereochemical designations familiar to one skilled in the art.

| | |
|---|---|
| AcCl | acetyl chloride |
| AcOH | acetic acid |
| AIBN | Azobisisobutyronitrile |
| BHFFT | bis(tetramethylene)fluoroformadmidinium |
| | hexafluorophosphate |
| Boc | tert-butyloxycarbonyl |
| BuLi | butyl lithium |
| DAST | Diethylaminosulfur trifluoroide |
| DCE | Dichloroethane |
| DCM | Dichloromethane |
| DIEA | diispropyl ethylamine |
| DMAP | 4-dimethylamino pyridine |
| DMF | Dimethylformamide |
| DPPA | Diphenyl phosphorylazide |
| Et₂O | diethyl ether |
| EtOAc | Ethyl acetate |
| EtOH | Ethanol |
| HATU | (1-[Bis(dimethylamino)methylene]-1H-1,2,3-triazolo[4,5- |
| | b]pyridinium 3-oxid hexafluorophosphate) |
| HMPA | hexamethylphosphoramide |
| IPA | isopropanol |
| i-Pr | Isopropyl |
| KHMDS | potassium bis(trimethylsilyl)amide |
| LDA | lithium diisopropyl amide |
| MeCN | Acetonitrile |
| MeOH | Methanol |
| Me | Methyl |
| NBS | N-bromosuccinimide |
| Pd/C | palladium on carbon |
| pTsOH | p-toluenesulfonic acid |
| PyBroP | Bromotripyrrolidinophosphonium hexafluorophosphate |
| T3P | 2,4,6-Tripropyl-1,3,5,2,4,6-trioxatriphosphorinane- |
| | 2,4,6-trioxide |
| TBAF | tetra-n-butyl ammonium fluoride |
| t-Bu | tert-butyl |
| Teoc | 2-(trimethylsilyl)ethyl carboxylate |
| TFA | trifluoro acetic acid |
| TFAA | trifluoro acetic anhydride |
| THF | Tetrahydrofuran |
| TsOH | Tolulenesulfonic acid |
| XPhos-Pd-G2 | 2nd generation XPhos precatalyst CAS no. 1310584-14-5 |

The following methods were used in the exemplified examples, except where noted otherwise. Purification of intermediates and final products was carried out via either normal or reverse phase chromatography. Normal phase chromatography was carried out using prepacked SiO₂ cartridges eluting with either gradients of hexanes and ethyl acetate or DCM and MeOH unless otherwise indicated. Reverse phase preparative HPLC was carried out using C18 columns with UV 220 nm or prep LCMS detection eluting with gradients of Solvent A (90% water, 10% MeOH, 0.1% TFA) and Solvent B (10% water, 90% MeOH, 0.1% TFA) or with gradients of Solvent A (95% water, 5% ACN, 0.1% TFA) and Solvent B (5% water, 95% ACN, 0.1% TFA) or with gradients of Solvent A (95% water, 2% ACN, 0.1% HCOOH) and Solvent B (98% ACN, 2% water, 0.1% HCOOH) or with gradients of Solvent A (95% water, 5% ACN, 10 mM NH₄OAc) and Solvent B (98% ACN, 2% water, 10 mM NH₄OAc) or with gradients of Solvent A (98% water, 2% ACN, 0.1% NH₄OH) and Solvent B (98% ACN, 2% water, 0.1% NH₄OH). LC/MS methods employed in characterization of examples are listed below.

### Method A:

Instrument: Waters Acquity coupled with a Waters MICROMASS® ZQ Mass Spectrometer
Linear gradient of 2 to 98% B over 1 min, with 0.5 min hold time at 98% B
UV visualization at 220 nm
Column: Waters BEH C18, 2.1×50 mm
Flow rate: 0.8 mL/min (Method A)
Mobile Phase A: 0.05% TFA, 100% water
Mobile Phase B: 0.05% TFA, 100% acetonitrile

### Method B:

Instrument: Shimadzu Prominence HPLC coupled with a Shimadzu LCMS-2020 Mass Spectrometer
Linear gradient of 0 to 100% B over 3 min, with 0.75 min hold time at 100% B
UV visualization at 220 nm
Column: Waters Xbridge C18, 2.1×50 mm, 1.7 um particles
Flow rate: 1 ml/min
Mobile Phase A: 10 mM ammonium acetate, 95:5 water:acetonitrile
Mobile Phase B: 10 mM ammonium acetate, 5:95 water:acetonitrile

### Method C:

Instrument: Shimadzu Prominence HPLC coupled with a Shimadzu LCMS-2020 Mass Spectrometer
Linear gradient of 0 to 100% B over 3 min, with 0.75 min hold time at 100% B
UV visualization at 220 nm
Column: Waters Xbridge C18, 2.1×50 mm, 1.7 um particles
Flow rate: 1 ml/min
Mobile Phase A: 0.1% TFA, 95:5 water:acetonitrile
Mobile Phase B: 0.1% TFA, 5:95 water:acetonitrile

### Method D:

Instrument: Waters Acquity coupled with a Waters MICROMASS® ZQ Mass Spectrometer
Linear gradient of 10% B to 98% B over 1 min, with 0.5 min hold time at 98% B
UV visualization at 220 nm
Column: Waters Acquity GEN C18, 2.1×50 mm, 1.7 um particles
Flow rate: 1 mL/min
Mobile Phase A: 0.05% TFA, 100% water
Mobile Phase B: 0.05% TFA, 100% acetonitrile

NMR Employed in Characterization of Examples. 1H NMR spectra were obtained with Bruker or JEOL® Fourier transform spectrometers operating at frequencies as follows: 1H NMR: 400 MHz (Bruker or JEOL-1) or 500 MHz (Bruker or JEOL®). Spectra data are reported in the format: chemical shift (multiplicity, coupling constants, number of hydrogens). Chemical shifts are specified in ppm downfield of a tetramethylsilane internal standard (d units, tetramethylsilane=0 ppm) and/or referenced to solvent peaks, which in 1H NMR spectra appear at 2.51 ppm for DMSO-d6, 3.30 ppm for CD₃OD, 1.94 ppm for CD₃CN, and 7.24 ppm for CDCl₃.

### Preparation of Intermediates:

### Intermediate 1-1: 5′-(tert-butoxycarbonyl)-2′-fluoro-4-methoxy-[1,1′-biphenyl]-3-carboxylic acid

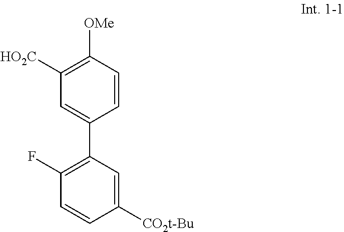 [SMILES: COc1ccc(-c2cc(C(=O)OC(C)(C)C)ccc2F)cc1C(=O)O]

To a vial was added 5-borono-2-methoxybenzoic acid (0.50 g, 2.6 mmol), tert-butyl 3-bromo-4-fluorobenzoate (0.84 g, 3.1 mmol), K₂CO₃ (1.76 g, 12.8 mmol), PdCl₂(dppf)-CH₂Cl₂ adduct (0.31 g, 0.38 mmol), and THF (22 mL). The reaction mixture was degassed for 2 min with nitrogen, then heated at 80° C. for 18 h. After allowing to cool to rt, the reaction mixture was diluted with IN HCl (25 mL) and the solution extracted with EtOAc (3×25 mL). The combined organic portions were dried over Na₂SO₄, filtered, concentrated under reduced pressure, and the resulting residue was dissolved in DMF and purified by preparative RP-HPLC to afford intermediate 1-1 (586 mg, 66.0% yield). LC-MS: RT=1.02 min; (M+H)⁺=347.1; [Method A].

Intermediate 2-6: 5′-(2-(tert-butoxy)-1-hydroxy-2-oxoethyl)-2′-fluoro-4-methoxy-[1,1′-biphenyl]-3-carboxylic acid. Intermediate 2-6 was prepared following the method outlined in the scheme below.

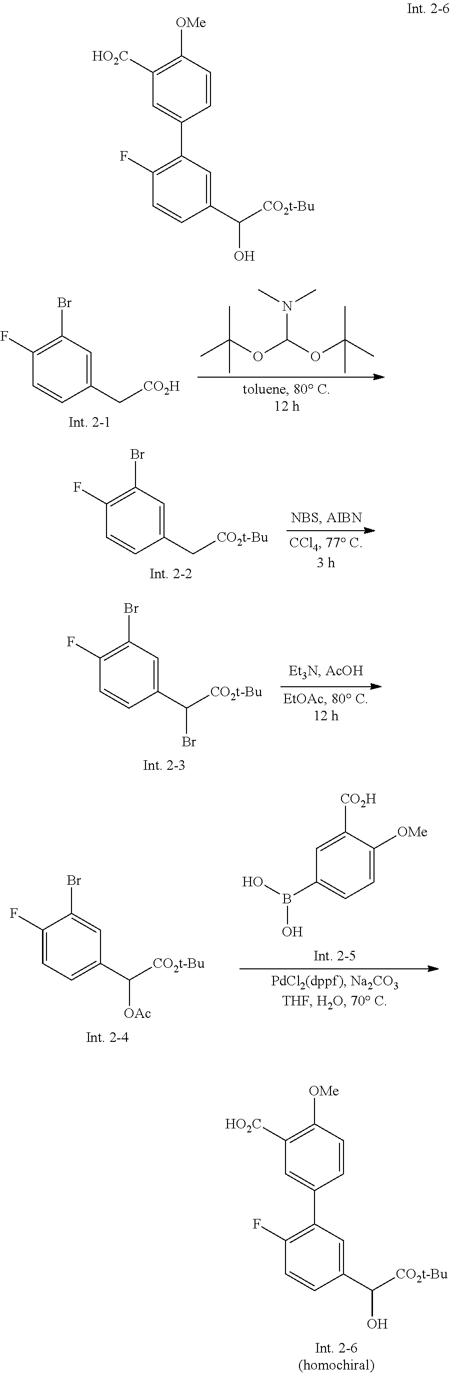

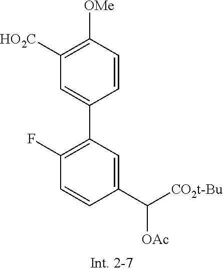 [SMILES: COc1ccc(-c2cc(C(OC(C)=O)C(=O)OC(C)(C)C)ccc2F)cc1C(=O)O]

Intermediate 2-2: Intermediate 2-2 was prepared employing known conditions for analogous substrates (Ludwig, J., Lehr, M. Syn. Comm. 2004, 34, 3691-3695), except the reaction temperature was maintained at 80° C. for 12 h. ¹H NMR (500 MHz, CDCl₃) δ 7.49 (dd, J=6.6, 2.2 Hz, 1H), 7.20 (ddd, J=8.3, 4.6, 2.2 Hz, 1H), 7.13-7.03 (m, 1H), 3.49 (s, 2H), 1.46 (s, 9H).

Intermediate 2-3: To a 20 mL reaction vial charged with intermediate 2-2 (0.27 g, 0.92 mmol) was added NBS (0.20 g, 1.1 mmol), CCL (10 mL), and AIBN (15 mg, 0.090 mmol). The solution was stirred at 77° C. for 3 h. The solution was concentrated under reduced pressure and purified by normal phase silica gel chromatographyto give intermediate 2-3 (310 mg, 0.84 mmol, 91% yield). ¹H NMR (500 MHz, CDCl₃) S 7.79 (dd, J=6.5, 2.3 Hz, 1H), 7.55-7.46 (m, 1H), 7.18-7.10 (m, 11H), 5.18 (s, 1H), 1.50 (s, 9H).

Intermediate 2-4: To a 2 dram vial charged with intermediate 2-3 was added EtOAc (2 mL), TEA (0.27 mL, 2.0 mmol), and acetic acid (0.1 mL, 2 mmol). The reaction mixture was stirred at 80° C. for 12 h. The reaction mixture was concentrated under reduced pressure and purified by normal phase silica gel chromatography to give intermediate 2-4 which was used without further purification. ¹H NMR (500 MHz, CDCl₃) δ 7.70 (dd, J=6.6, 2.2 Hz, 1H), 7.41 (ddd, J=8.4, 4.7, 2.1 Hz, 1H), 7.15 (t, J=8.4 Hz, 1H), 5.77 (s, 1H), 2.22 (s, 3H), 1.43 (s, 9H).

Intermediate 2-6: Intermediate 2-6 was prepared from intermediate 2-4, employing 5-borono-2-methoxybenzoic acid 2-5 using similar conditions to those described for intermediate 1-1. After reverse phase HPLC (using Phenomenex Luna C18 5u 30×100 mm column, 10-minute gradient; Solvent A: 10% ACN-90% H₂O-0.1% TFA; Solvent B: 90% ACN-10% H₂O-0.1% TFA), half of the material was isolated as intermediate 2-7 (85 mg, 0.60 mmol, 34% yield); ¹H NMR (500 MHz, CDCl₃) δ 8.43-8.36 (m, 1H), 7.81 (dt, J=8.7, 2.0 Hz, 1H), 7.56 (dd, J=7.3, 2.3 Hz, 1H), 7.45 (ddd, J=8.5, 4.6, 2.3 Hz, 1H), 7.23-7.16 (m, 2H), 5.84 (s, 1H), 4.17 (s, 3H), 2.23 (s, 3H), 1.45 (s, 9H) while the other half was isolated as the alcohol intermediate 2-6 (70 mg, 0.19 mmol, 31% yield); ¹H NMR (500 MHz, CDCl₃) δ 8.40 (d, J=2.2 Hz, 1H), 7.82 (dt, J=8.6, 2.2 Hz, 1H), 7.54 (dd, J=7.4, 2.5 Hz, 1H), 7.41 (ddd, J=8.4, 4.8, 2.2 Hz, 1H), 7.19-7.14 (m, 2H), 5.09 (s, 1H), 4.16 (s, 3H), 1.47 (s, 9H). Intermediate 2-6 was separated into individual enantiomers using chiral SFC. Preparative chromatographic conditions: Instrument: Berger MG II; Column: Chiralpak ID, 21×250 mm, 5 micron; Mobile phase: 25% IPA/75% CO₂; Flow conditions; 45 mL/min, 120 Bar, 40° C.; Detector wavelength: 220 nm; Injection details: 8 injections of 0.36 mL of ~20 mg/mL in IPA. Analytical chromatographic conditions: Instrument: Waters UPC2 analytical SFC; Column: Chiralpak ID 4.6×100 mm, 3 micron; Mobile phase: 25% IPA/75% CO₂: Flow conditions: 2 mL/min, 150 Bar, 40° C.: Detector wavelength: 220 nm. Peak 1, RT=3.89 min, >99.5% ee: Peak 2, RT=5.44 min, >99.5% ee. as intermediate 2-6.

Intermediate 3-2: 5′-(2-(tert-butoxy)-1-((tert-butoxycarbonyl)amino)-2-oxoethyl)-2′-fluoro-4-methoxy-[1,1-biphenyl]-3-carboxylic acid. The titled compound was prepared following the method outlined in the scheme below.

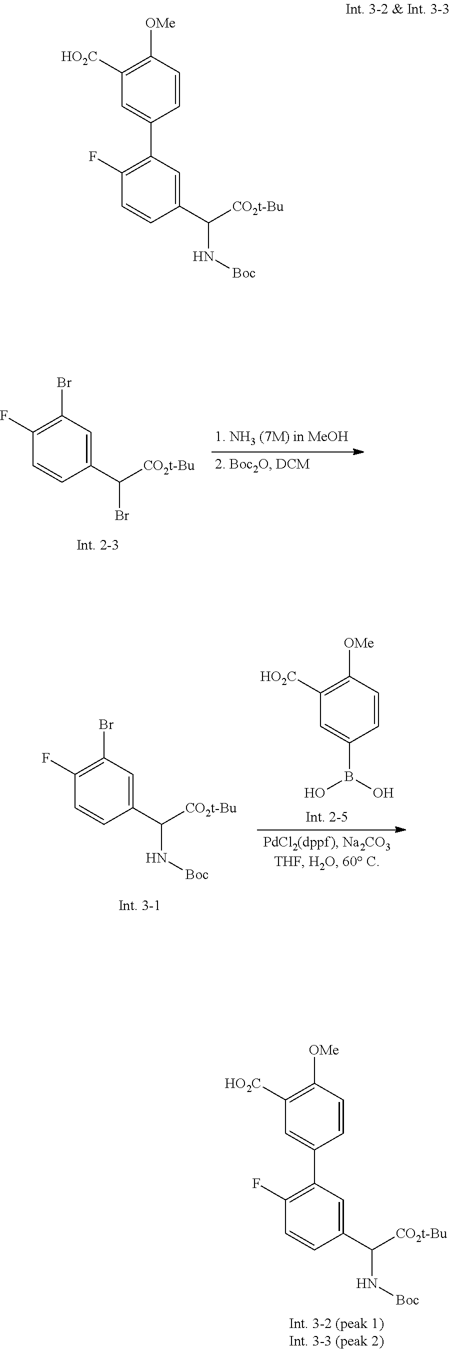 [SMILES: COc1ccc(-c2cc(C(NC(=O)OC(C)(C)C)C(=O)OC(C)(C)C)ccc2F)cc1C(=O)O ; CC(C)(C)OC(=O)C(Br)c1ccc(F)c(Br)c1 ; CC(C)(C)OC(=O)NC(C(=O)OC(C)(C)C)c1ccc(F)c(Br)c1 ; COc1ccc(B(O)O)cc1C(=O)O ; COc1ccc(-c2cc(C(NC(=O)OC(C)(C)C)C(=O)OC(C)(C)C)ccc2F)cc1C(=O)O]

Intermediate 3-1: To 2-3 (60 mg, 0.16 mmol) was added ammonia (0.5 mL, 3.5 mmol, in MeOH). After stirring at rt for 12 h, the mixture was concentrated under vacuum. To the amine in DCM (1 mL) was added BOC-anhydride (0.11 mL, 0.49 mmol) and DIEA (57 μL, 0.33 mmol) and the reaction mixture was stirred at rt for 1 h. The mixture was concentrated under vacuum and silica gel chromatography purification produced 3-1 (42 mg, 0.1 mmol, 63% yield). LC-MS: RT=1.14 min: MS (EST) m/z=406.0 (M+H)⁺; [Method A].

Intermediates 3-2 and 3-3: Intermediates 3-2 and 3-3 were prepared employing that similar Suzuki cross coupling conditions that were used for intermediate 1-1, except at a temperature of 60° C. for 18 h. After allowing to cool to rt, the reaction mixture was diluted with 1N HCl (25 mL) and the solution extracted with EtOAc (3×25 mL). The combined organic portions were dried over Na₂SO₄, filtered, concentrated under reduced pressure, and purified by preparative RP-HPLC. ¹H NMR (500 MHz, CDCl₃) δ 8.38 (d, J=1.9 Hz, 1H), 7.80 (dt, J=8.7, 2.0 Hz, 1H), 7.46 (dd, J=7.4, 2.5 Hz, 1H), 7.36 (dddd, J=8.8, 4.4, 2.2, 1.1 Hz, 1H), 7.19-7.13 (m, 2H), 5.67 (br d, J=5.2 Hz, 1H), 5.25 (br d, J=6.3 Hz, 1H), 4.16 (s, 3H), 1.46 (br s, 9H), 1.44 (s, 9H). The residue was separated into individual enantiomers using chiral SFC. Preparative chromatographic conditions: Instrument: Berger MG II; Column: Chiralpak 1D, 21×250 mm, 5 micron; Mobile phase: 20% MeOH/80% CO₂: Flow conditions: 45 mL/min, 120 Bar, 40° C.: Detector wavelength: 209 nm: Injection details: 49 injections in MeOH. Analytical chromatographic conditions: Instrument: Waters UPC2 analytical SFC; Column: Chiralpak 1C, 4.6×100 mm, 3 micron; Mobile phase: 25% MeOH/75% CO₂; Flow conditions: 2 mL/min, 150 Bar, 40° C.: Detector wavelength: 220 nm. 3-2, Peak 1, RT=4.22 min, 95.7% ee; 3-3, Peak 2, RT=5.11 min, >99% ee.

Intermediate 4-4: 2′-fluoro-4-methoxy-5′-(2,2,2-trifluoro-1-hydroxyethyl)-[1,1′-biphenyl]-3-carboxylic acid. The titled compound was prepared following the scheme outlined below.

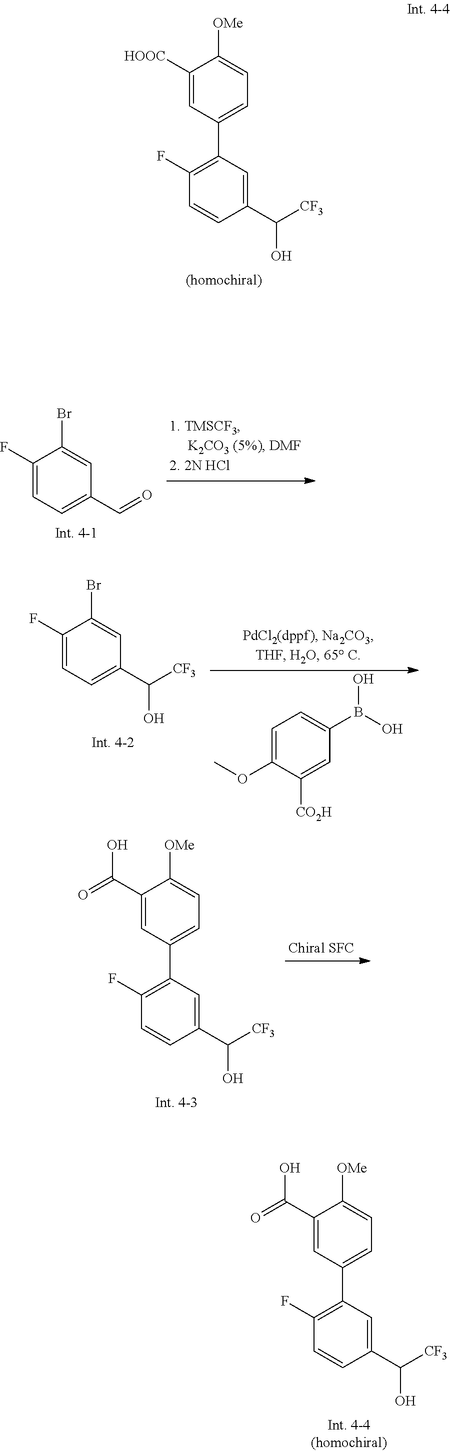 [SMILES: COc1ccc(-c2cc(C(O)C(F)(F)F)ccc2F)cc1C(=O)O ; O=Cc1ccc(F)c(Br)c1 ; OC(c1ccc(F)c(Br)c1)C(F)(F)F ; COc1ccc(B(O)O)cc1C(=O)O ; COc1ccc(-c2cc(C(O)C(F)(F)F)ccc2F)cc1C(=O)O ; COc1ccc(-c2cc(C(O)C(F)(F)F)ccc2F)cc1C(=O)O]

Intermediate 4-2: Into the reaction vessel was added 3-bromo-4-fluorobenzaldehyde (4-1, 235 mg, 1.15 mmol), DMF (3.5 mL), (trifluoromethyl)trimethylsilane (0.34 mL, 2.3 mmol), and K₂CO₃ (8 mg, 0.06 mmol). The reaction mixture was stirred at rt for 60 min, the reaction mixture allowed to cool to it and 2N HCl (3 mL) was added. After stirring at rt for an additional 1 h, the reaction mixture was diluted with EtOAc (15 mL), and the solution washed with sat NH₄Cl. The aqueous phase was extracted with EtOAc (2×10 mL). The combined organic portions were dried over Na₂SO₄, filtered, concentrated under reduced pressure, and purified by silica gel chromatography (0-35% EtOAc in hexanes) to produce 4-2 (205 mg, 0.75 mmol, 65% yield). ¹H NMR (500 MHz, CDCl₃) d 7.74 (dd, J=6.5, 2.1 Hz, 1H), 7.43 (ddd, J=8.4, 4.8, 2.2 Hz, 1H), 7.19 (t, J=8.4 Hz, 1H), 5.11-4.98 (m, 1H), 2.69 (d, J=4.4 Hz, 1H).

Intermediate 4-3: Into the reaction vessel containing 4-2 (100 mg, 0.37 mmol) was added 5-borono-2-methoxybenzoic acid (93 mg, 0.48 mmol), PdCl₂(dppf)-CH₂Cl₂ adduct (50 mg, 0.06 mmol), Na₂CO₃ (155 mg, 1.46 mmol), and H₂O (1 mL). The reaction mixture was degassed by bubbling N₂ for 10 min, scaled, and stirred at 65° C. for 3 h. After allowing to cool to rt, the reaction mixture was quenched by the addition of IN HCl, the solution extracted with EtOAc, dried over Na₂SO₄, filtered, concentrated under reduced pressure and purified by HPLC to produce 4-3 (51 mg, 0.15 mmol, 40% yield). ¹H NMR (500 MHz, CDCl₃) δ 8.39 (d, J=1.9 Hz, 1H), 7.83 (dt, J=8.7, 2.1 Hz, 1H), 7.59 (dd, J=7.3, 2.1 Hz, 1H), 7.53-7.45 (m, 1H), 7.23 (dd, J=10.2, 8.8 Hz, 1H), 7.18 (d, J=8.5 Hz, 1H), 5.11 (q, J=6.6 Hz, 1H), 4.17 (s, 3H). MS (ESI) m/z=345.1 (M+H)⁺.

Intermediate 4-4: Intermediate 4-3 was separated into individual enantiomers using chiral SFC. Preparative chromatographic conditions: Instrument: Berger MG II; Column: Kromasil 5-CelluCoat, 21×250 mm, 5 micron: Mobile phase: 15% IPA-ACN (0.1% DEA)/85% CO₂; Flow conditions: 45 mL/min, 120 Bar, 40° C.; Detector wavelength: 220 nm: Injection details: 0.4 mL of ~5 mg/mL in ACN-IPA (1:1). Peak 2 was collected to afford intermediate 4-4. Analytical chromatographic conditions: Instrument: Aurora Infinity Analytical SFC; Column: Kromasil 5-CelluCoat, 4.6×250 mm, 5 micron; Mobile phase: 20% IPA-ACN (0.1% DEA)/80% CO₂; Flow conditions: 2 mL/min, 150 Bar, 40° C.; Detector wavelength: 220 nm. Peak 1, RT=9.12 min, 99% ee; Peak 2, RT=10.19 min, 98% ee.

Intermediate 5-2: 5-(5-hydroxy-3a,5,6,6a-tetrahydro-4H-cyclopenta[d]isoxazol-3-yl)-2-methoxybenzoic acid. The titled compound was prepared following the scheme outlined below.

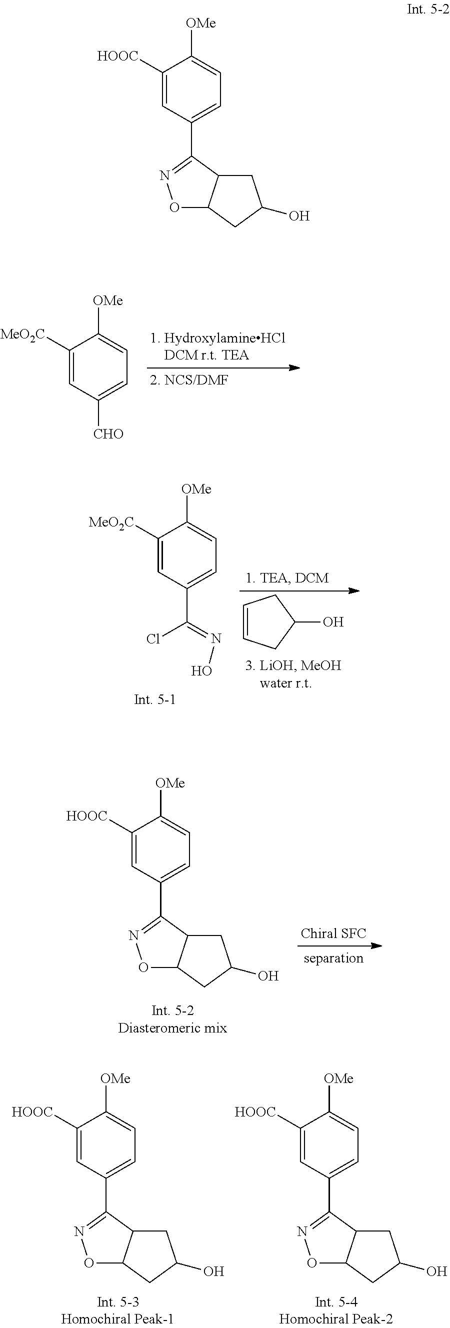

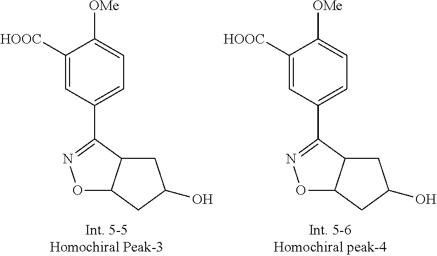 [SMILES: COc1ccc(C2=NOC3CC(O)CC23)cc1C(=O)O ; COc1ccc(C2=NOC3CC(O)CC23)cc1C(=O)O]

Intermediate 5-1. Methyl 5-formyl-2-methoxybenzoate (24.9 g, 128 mmol) was dissolved in DCM (500 mL). To the solution was added triethylamine (17.9 mL, 128 mmol) followed by hydroxylamine hydrochloride (8.91 g, 128 mmol). The reaction mixture was stirred for 14 h at rt and concentrated under reduced pressure to yield a white solid. The solid was dissolved in water (200 mL) and the aqueous portion extracted with EtOAc (2×100 mL). The combined organic portion was, dried (MgSO₄), filtered and concentrated under reduced pressure to yield a white solid (27.1 g, 100% yield). The solid was re-dissolved in DMF (200 mL) and to the solution was added NCS (17.2 g, 128 mmol) and stirred at rt for 14 h overnight. The reaction mixture was quenched by the addition of excess water and a white solid precipitated. The solid was separated by filtration and washed with excess water and dried under vacuum to afford a white solid (28.7 g, 89% yield) for intermediate 5-1. ¹H NMR (500 MHz, CDCl₃) δ 8.32-8.30 (m, 1H), 7.99-7.96 (m, 1H), 7.80-7.78 (m, 1H), 7.05-7.02 (d, 1H), 3.98 (s, 3H), 3.94 (s, 3H).

Intermediate 5-2 (diastereomeric mixture): Alternatively, (F)-5-((hydroxyimino)methyl)-2-methoxybenzoic acid (620 mg, 3.18 mmol) was dissolved in DMF (5 mL), to the solution was added NCS (424 mg, 3.18 mmol) and the reaction mixture was stirred at rt for 4 h. The reaction mixture was quenched with the addition of water (100 mL) and the solution extracted with EtOAc (2×25 mL), dried (MgSO₄) and evaporated under reduced pressure to an oil. The oil obtained was re-dissolved in DCM (10 mL) and cyclopent-3-ene-1-ol (2.67 g, 31.8 mmol) was added, followed by the addition of TEA (0.44 mL) and the reaction mixture stirred at rt for 14 h The resulting solution was filtered through a plug of silica gel and concentrated under reduced pressure to afford the diasteromeric mixture of intermediate 5-2. ¹H NMR (600 MHz, CDCl₃) o 8.04 (d, J=2.3 Hz, 1H), 7.85 (dd, J=8.8, 2.3 Hz, 1H), 7.03 (d, J=8.8 Hz, 1H), 5.30 (ddd, J=9.4, 6.2, 2.9 Hz, 1H), 4.50 (quin, J=5.9 Hz, 1H), 4.19 (td, J=9.3, 4.7 Hz, 1H), 3.92 (s, 3H), 2.33-2.27 (m, 1H), 2.18-2.06 (m, 3H). LC-MS: RT=0.83 min; MS (ESI) m/z=278.1 (M+H)⁺; [Method A].

The chiral intermediates of 5-2 were separated by chiral SFC by the following preparative chromatographic methods: Instrument: Berger SFC; Column: IC 25×3 cm ID, 5 μm, Temperature: 40C, Flow rate: 85 mL/min, Mobile Phase: gradient 75/25 CO₂/MeOH for 12 min then to 45% MeOH, Detector Wavelength: 235 nm, Injection Volume: 1000 μL to afford chiral 5-3 Peak-1, >99% ee, Analytical RT=8.80 min), chiral 5-4 (Peak-2, >95% ee, Analytical RT=9.86 min), chiral 5-5 (Peak-3, >99% cc, Analytical RT=13.53 min), chiral 5-6 (Peak-4, >99% cc, Analytical RT=16.67 min). Analytical Chromatographic Conditions: Instrument: Agilent SFC (LVL-L4021 Lab), Column: IC 250×4.6 mm ID, 5 μm, Temperature: Ambient, Flow rate: 2.0 mL/min, Mobile Phase: gradient 75/25 CO₂/MeOH 12 min then to 45% MeOH. Analytical data for peak-1-4: ¹H NMR (600 MHz, CD₃OD) δ 8.07 (d, J=2.2 Hz, 1H), 7.82 (dd, J=8.7, 2.1 Hz, 1H), 7.18 (d, J=8.8 Hz, 1H), 5.21 (ddd, J=9.2, 6.2, 2.5 Hz, 1H), 4.27 (m, 11H), 4.24 (td, J=9.4, 4.0 Hz, 1H), 3.94 (s, 3H), 2.16 (m, 1H), 2.05 (m, 1H), 2.00 (m, 1H), 1.99 (m, 11-).

Intermediate 6-2: Preparation of 5-(5-(hydroxymethyl)-3a,5,6,6a-tetrahydro-4H-cyclopenta[d]-isoxazol-3-yl)-2-methoxybenzoic acid.

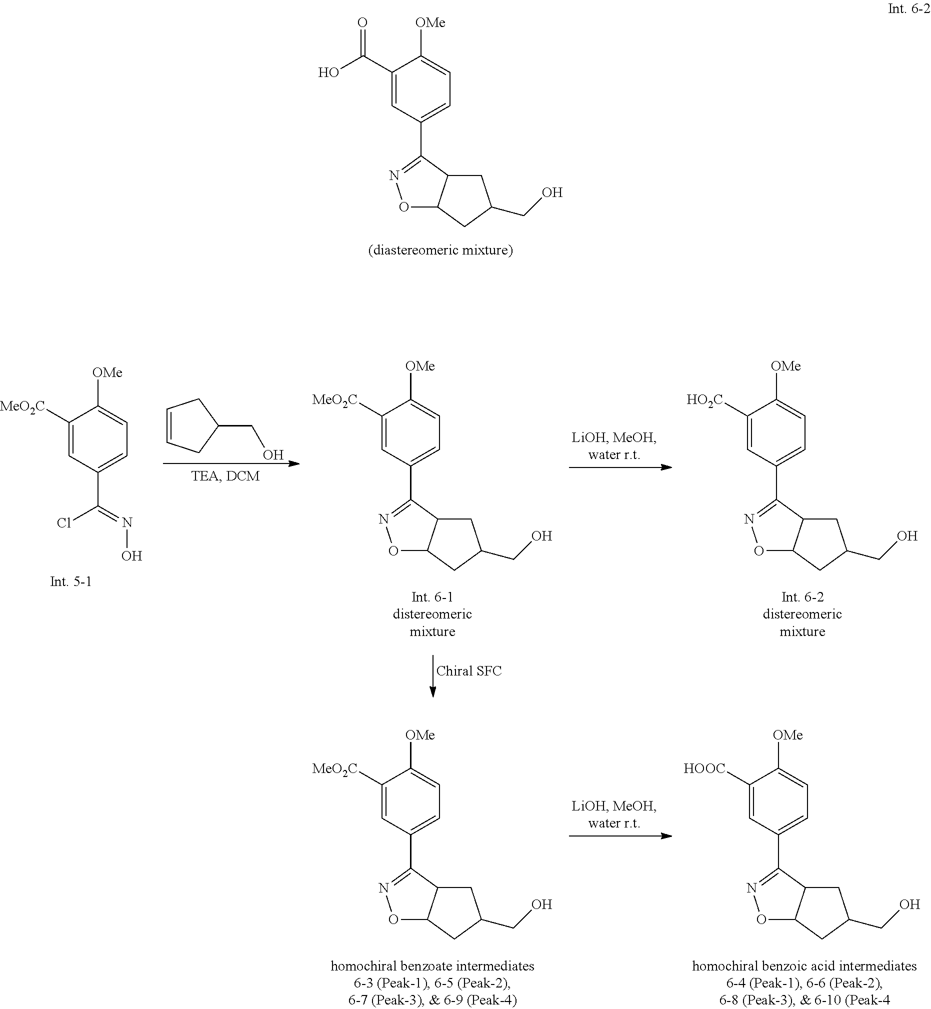

Intermediate 6-1: Intermediate 5-1 (3.0 g, 12.3 mmol) was dissolved in DCM (123.13 mL) and to this was added cyclopent-3-en-1-ylmethanol (4.8 g, 49.3 mmol) followed by the addition of TEA (5.15 mL, 36.9 mmol) and the reaction mixture was stirred at rt. After stirring 14 h, the reaction mixture was concentrated under reduced pressure and the residue purified by normal phase chromatography by eluting with hexanes/EtOAc gave 6-1 (2.8 g, 9.2 mmol, 75% yield) as an oily mass. LC-MS: RT=0.95 min; MS (ESI) m/z=306.3 (M+H)⁺; [Method A]

Diasteromeric intermediate 6-2: Intermediate 6-1 (88 mg, 0.29 mmol) was dissolved in THF (1 mL)/MeOH (1 mL) was treated with LiOH monohydrate (36 mg, 0.86 mmol) in H₂O (1 mL) at rt. After 3 h, the reaction mixture was diluted with H₂O (5 mL) and the pH of the aq. layer was adjusted to pH 7 with 1M HCl solution and extracted with EtOAc (2×25 mL), washed with brine, dried (Na₂SO₄), filtered, and evaporated under reduced pressure to give 6-2 (62 mg, 74% yield). The carboxylic acid 6-2 was carried forward to the next reaction without further purification. LC-MS: RT=0.85 min; MS (ESI) m/z=292.3 (M+H)⁺; [Method A].

### Homochiral Intermediates 6-3 Through 6-10

Individual chiral diastereomer ester intermediates 6-3, 6-5, 6-7, and 6-9 were obtained by chiral SFC separation of the diastereomeric mixture intermediate 6-1 (525 mg, 1.72 mmol). Chiral SFC Preparative chromatographic conditions: Instrument: Berger MG II (SFC); Column: Chiralpak AD-H, 21×250 mm, 5 micron: Mobile phase: 15% MeOH/85% CO₂: Flow conditions: 45 mL/min, 150 Bar, 40° C.; Detector wavelength: 210 nm; Injections details: 0.5 mL of −35 mg/mL in MeOH. Analytical chromatographic conditions: Instrument: Shimadzu Nexera SFC: Column: Chiralpak AD-H, 4.6×100 mm, 3 micron; Mobile phase: 15% MeOH/85% CO₂; Flow conditions: 2.0 mL/min, 150 Bar, 40° C.; Detector wavelength: 220 nm: Injection details: 5 μL of ~1 mg/mL in MeOH.

Homochiral methylbenzoate intermediate 6-3 (Peak-1, RT=4.07 min; >99% ee) was obtained as a film (150 mg, 29% yield). ¹H NMR (600 MHz, CDCl₃) δ 8.04 (d, J=2.3 Hz, 1H), 7.87 (dd, J=8.7, 2.3 Hz, 1H), 7.01 (d, J=8.8 Hz, 1H), 5.23 (dd, J=8.8, 5.1 Hz, 1H), 4.10 (t, J=8.7 Hz, 1H), 3.94 (s, 3H), 3.90 (s, 3H), 3.72-3.66 (m, 1H), 3.61 (dt, J=10.5, 5.2 Hz, 1H), 2.30-2.16 (m, 2H), 2.05 (dd, J=13.0, 6.1 Hz, 1H), 1.76 (ddd, J=12.9, 11.5, 9.4 Hz, 1H), 1.68-1.62 (m, 1H), 1.39 (br t, J=4.8 Hz, 1H).

Homochiral benzoic acid intermediate 6-4 (Peak-1): Preparation of 5-(5-(hydroxymethyl)-3a,5,6,6a-tetrahydro-4H-cyclopenta[d]isoxazol-3-yl)-2-methoxybenzoic acid. Intermediate 6-4 (100 mg, 78% yield) was prepared in a similar manner as intermediate 6-2 with the hydrolysis of homochiral intermediate 6-3 (Peak-1). LC-MS: RT=0.85 min: MS (EST) m/z=292.3 (M+H)⁺; [Method A].

Homochiral methylbenzoate intermediate 6-5 (Peak-2, RT=4.55 min; >99% ee) was obtained as a film (33.2 mg, 6.3% yield). ¹H NMR (600 MHz, CDCl₃) δ 8.05 (d, J=2.3 Hz, 1H), 7.87 (dd, J=8.8, 2.3 Hz, 1H), 7.02 (d, J=8.8 Hz, 1H), 5.25 (ddd, J=10.1, 6.2, 4.2 Hz, 1H), 4.04-3.98 (m, 1H), 3.94 (s, 3H), 3.90 (s, 3H), 3.63-3.57 (m, 1H), 3.56-3.50 (m, 1H), 2.38-2.26 (m, 3H), 1.92-1.85 (m, 1H), 1.73-1.66 (m, 11H), 1.51 (t, J=5.3 Hz, 1H).

Homochiral benzoic acid intermediate 6-6 (Peak-2): Preparation of 5-(5-(hydroxymethyl)-3a,5,6,6a-tetrahydro-4H-cyclopenta[d]isoxazol-3-yl)-2-methoxybenzoic acid. Intermediate 6-6 (20.2 mg, 92% yield) was prepared in a similar manner as intermediate 6-2 with the hydrolysis of intermediate 6-5 (Peak-2). LC-MS: RT=0.83 min; MS (ESI) m/z=292.3 (M+H)⁺; [Method A].

Homochiral methylbenzoate intermediate 6-7 (Peak-3, RT=5.66 min; >99% ee) was obtained as a film (161 mg, 30.6% yield). ¹H NMR: (600 MHz, CDCl₃) δ 8.05-8.03 (m, 1H), 7.86 (dd, J=8.7, 2.3 Hz, 1H), 7.01 (d, J=8.8 Hz, 1H), 5.23 (dd, J=8.7, 5.2 Hz, 1H), 4.10 (t, J=8.7 Hz, 1H), 3.94 (s, 3H), 3.90 (s, 3H), 3.69 (br dd, J=10.6, 5.2 Hz, 1H), 3.63-3.58 (m, 1H), 2.28-2.17 (m, 2H), 2.05 (br dd, J=12.9, 6.2 Hz, 1H), 1.76 (ddd, J=13.0, 11.5, 9.4 Hz, 1H), 1.64-1.60 (m, 1H), 1.49 (br s, 1H).

Homochiral benzoic acid intermediate 6-8 (Peak-3): Preparation of 5-(5-(hydroxymethyl)-3a,5,6,6a-tetrahydro-4H-cyclopenta[d]isoxazol-3-yl)-2-methoxybenzoic acid. Intermediate 6-8 (120 mg, 85% yield) was prepared in a similar manner as intermediate 6-2 with the hydrolysis of intermediate 6-7 (Peak-3). LC-MS: RT=0.83 min; MS (ESI) m/z=292.3 (M+H)⁺; [Method A].

Homochiral methylbenzoate intermediate 6-9 (Peak-4, RT=9.81 min; >99% ee) was obtained as a film (47 mg, 9.0% yield). ¹H NMR: (600 MHz, CDCl₃) δ 8.04 (d, J=2.3 Hz, 1H), 7.87 (dd, J=8.7, 2.3 Hz, 1H), 7.02 (d, J=8.8 Hz, 1H), 5.24 (ddd, J=10.1, 6.2, 4.2 Hz, 1H), 4.03-3.98 (m, 11H), 3.94 (s, 3H), 3.90 (s, 3H), 3.63-3.57 (m, 1H), 3.56-3.49 (m, 1H), 2.38-2.25 (m, 3H), 1.91-1.85 (m, 1H), 1.72-1.66 (m, 1H), 1.55 (br s, 1H).

Homochiral benzoic acid intermediate 6-10 (Peak-4). Preparation of 5-(5-(hydroxymethyl)-3a,5,6,6a-tetrahydro-4H-cyclopenta[d]isoxazol-3-yl)-2-methoxybenzoic acid. Intermediate 6-10 (18.2 mg, 52% yield) was prepared in a similar manner as intermediate 6-2 with the hydrolysis of intermediate 6-9 (Peak-4). LC-MS: RT=0.84 min; MS (ESI) m/z=292.3 (M+H)⁺; [Method A].

Intermediate 7-6: 3-amino-N-(4-fluoro-3-(trifluoromethyl)phenyl)-5,6,7,8-tetrahydro-naphthalene-2-carboxamide. The tided compound was prepared following the method outlined in the scheme below.

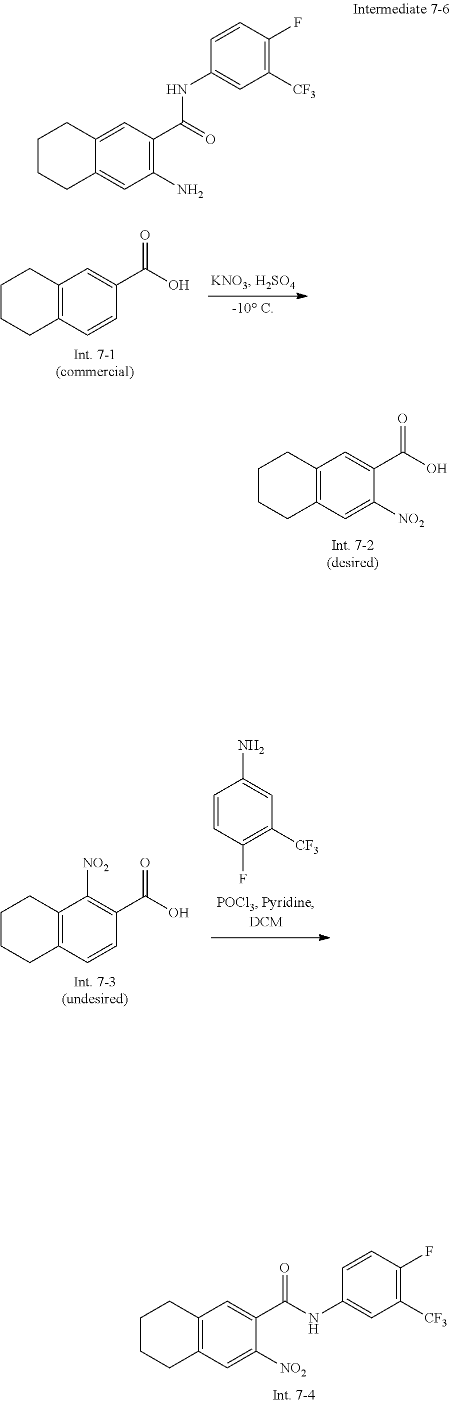 [SMILES: Nc1cc2c(cc1C(=O)Nc1ccc(F)c(C(F)(F)F)c1)CCCC2 ; O=C(O)c1ccc2c(c1)CCCC2 ; O=C(O)c1cc2c(cc1[N+](=O)[O-])CCCC2 ; O=C(O)c1ccc2c(c1[N+](=O)[O-])CCCC2 ; Nc1ccc(F)c(C(F)(F)F)c1 ; O=C(Nc1ccc(F)c(C(F)(F)F)c1)c1cc2c(cc1[N+](=O)[O-])CCCC2]

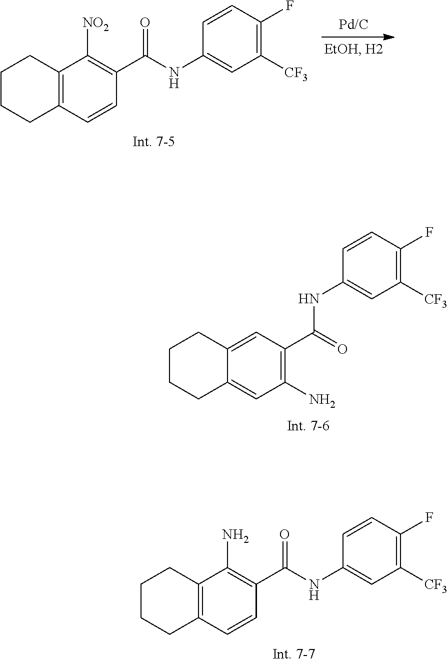 [SMILES: O=C(Nc1ccc(F)c(C(F)(F)F)c1)c1ccc2c(c1[N+](=O)[O-])CCCC2 ; Nc1cc2c(cc1C(=O)Nc1ccc(F)c(C(F)(F)F)c1)CCCC2 ; Nc1c(C(=O)Nc2ccc(F)c(C(F)(F)F)c2)ccc2c1CCCC2]

Intermediate 7-2 & 7-3: A solution of 5,6,7,8-tetrahydronaphthalene-2-carboxylic acid (2.0 g 11 mmol) in H₂SO₄ (20 mL) at 0° C. was treated dropwise with KNO₃ (1.38 g, 13.6 mmol) in H₂SO₄ (10 mL). After 12 h, the reaction mixture was quenched with ice and extracted with DCM (2×25 mL). The organic layer was washed with water, brine, dried over sodium sulfate, filtered, and carried forward to the next reaction without further purification as a mixture of regiosomers. LC-MS: RT=0.99 min: MS (ESI) m/z=221.9 (M+H)⁺; [Method A].

Intermediates 7-4 & 7-5: POCl₃ (1.05 mL, 11.3 mmol) in DCM (10 mL) was added via syringe to a solution of 7-2 and 7-3 (2.5 g, 11 mmol) and 4-fluoro-3-(trifluoromethyl) aniline (2.02 g, 22.6 mmol), and pyridine (7.3 mL, 90 mmol) in DCM (75 mL) at 0° C. After 4 h, the reaction mixture was quenched with 1.0 M HCl, the organic layer was separated and washed with 1.0 M HCl, water, brine, dried over sodium sulfate, filtered, concentrated under reduced pressure and purified by a normal phase chromatography by eluting with hexanes/EtOAc to give a mixture of two regioisomers 7-4 and 7-5 (2.88 g, 7.51 mmol, 66% yield) as an off white solid. LC-MS: RT=1.13 min: MS (ESI) m/z=382.9 (M+H)⁺; [Method A].

Intermediates 7-6 & 7-7: Pd—C (wet, Degussa type, 10%) (160 mg, 1.50 mmol) was added to a solution of 7-4 and 7-5 (2.88 g, 7.52 mmol) in EtOH (75.2 mL) and subjected to a hydrogen atmosphere (55 psi). After 3 h, the catalyst was filtered through a plug of Celite and the filtrate concentrated under reduced pressure. The two regioisomers were purified by SFC: Instrument: Column (Chiralpak AD-H, 21×250 mm, 5 micron); Mobile Phase (25% MeOH/75% CO₂): Flow Conditions (45 mL/min, 150 Bar, 40° C.); Detector Wavelength (220 nm): Injection Details (0.5 mL of ~80 mg/mL in MeOH).

Intermediate 7-6 (Peak 2, RT=5.68 min): 3-amino-N-(4-fluoro-3-(trifluoromethyl)phenyl)-5,6,7,8-tetrahydronaphthalene-2-carboxamide (580 mg, 22% yield). ¹H NMR (500 MHz, DMSO-d6) δ 10.19 (s, 1H), 8.18 (dd, J=6.6, 2.4 Hz, 1H), 8.01 (dt, J=8.0, 3.9 Hz, 1H), 7.48 (t. J=9.8 Hz, 1H), 7.36 (s, 1H), 6.46 (s, 1H), 6.07 (s, 2H), 2.68-2.58 (m, 4H), 1.77-1.64 (m, 4H). LCMS?

Intermediate 7-7 (Peak 1, RT=4.27 min): 1-amino-N-(4-fluoro-3-(trifluoromethyl)phenyl)-5,6,7,8-tetrahydronaphthalene-2-carboxamide (725 mg, 27% yield). ¹H NMR (500 MHz, DMSO-d6) δ 10.18 (s, 1H), 8.19 (dd, J=6.6, 2.7 Hz, 1H), 8.03 (ddd, J=8.9, 4.3, 2.9 Hz, 1H), 7.51-7.45 (m, 2H), 6.39 (d, J=8.3 Hz, 1H), 6.29 (s, 2H), 2.67 (t, J=6.1 Hz, 2H), 2.38 (t, J=6.4 Hz, 2H), 1.84-1.74 (m, 2H), 1.72-1.63 (m, 2H).

Intermediate 8-5: Preparation of 3-amino-N-(4-fluoro-3-(trifluoromethyl)phenyl)-5-oxo-5,6,7,8-tetrahydronaphthalene-2-carboxamide. The titled compound was prepared following the method outlined in the scheme below.

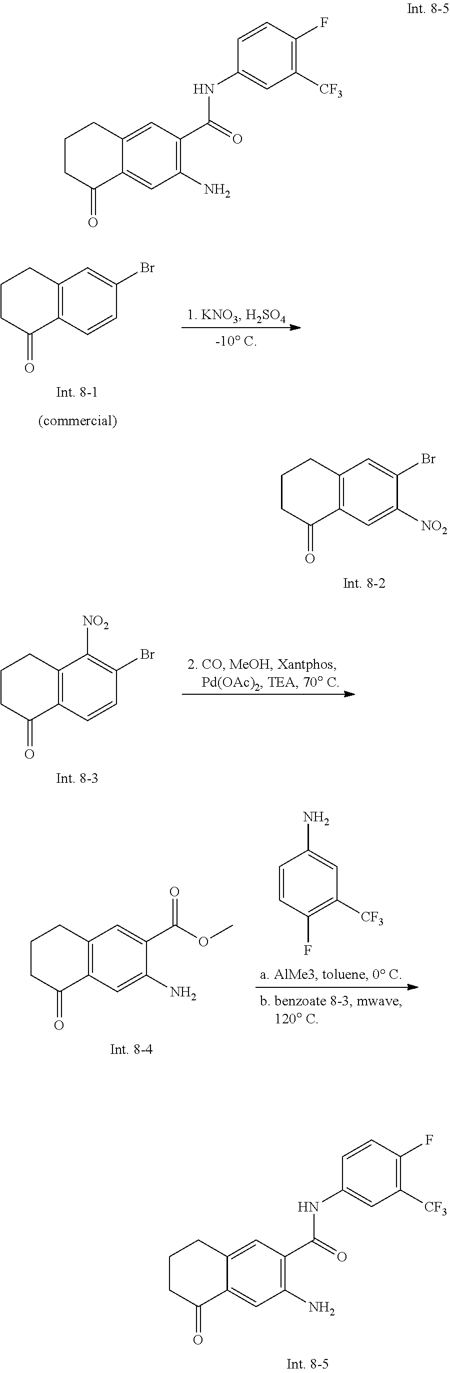

Intermediate 8-2: 6-bromo-3,4-dihydronaphthalen-1(2H)-one (Intermediate 8-1) (0.5 g, 2 mmol) was dissolved in H₂SO₄ (5 mL) at 0° C. After stirring for 5 min, KNO₃ (0.23 g, 2.2 mmol) in H₂SO₄ (1 mL) was introduced dropwise while maintaining the reaction mixture temperature below 15° C. and then allowed to gradually come to rt. After 12 h, the reaction mixture was quenched with ice, neutralized with saturated sodium bicarbonate solution and extracted with DCM (2×25 mL). The organic layer was washed with water, brine, dried over sodium sulfate, filtered, and concentrated under reduced pressure, purified by normal phase chromatography by eluting with hexanes/EtOAc to give a mixture of major regioisomer 6-bromo-7-nitro-3,4-dihydronaphthalen-1(2H)-one (Intermediate 8-2) (70% by ¹H NMR) and minor regioisomer 6-bromo-5-nitro-3,4-dihydronaphthalen-1(2H)-one (Intermediate 8-3) (30% by ¹H NMR) (540 mg, 90% yield). Intermediate 8-2 (major): ¹H NMR (600 MHz, DMSO-d6) δ: 8.32 (s, 1H), 8.02 (s, 1H), 3.02 (t, J=6.2 Hz, 2H), 2.70-2.64 (m, 4H), 2.13-2.04 (m, 4H). Intermediate 8-3 (minor): ¹H NMR (600 MHz, DMSO-d6) δ: 7.97 (d, J=8.4 Hz, 1H), 7.90 (d, J=8.5 Hz, 1H), 2.85 (t, J=6.1 Hz, 2H), 2.70-2.64 (m, 4H), 2.13-2.04 (m, 4H).

Intermediate 8-4: A solution of 6-bromo-7-nitro-3,4-dihydronaphthalen-1(2H)-one (intermediate 8-2) (500 mg, 1.85 mmol) in TEA (3.88 mL, 27.8 mmol), MeOH (0.90 mL, 22 mmol), and DMF (2 mL) was degassed with nitrogen and Pd(OAc)₂ (10.39 mg, 0.05000 mmol) and Xantphos (53.6 mg, 0.0900 mmol) were added. The solution was degassed and CO gas was bubbled through the solution. The reaction vessel was fitted with a reflux condenser and a CO balloon and then heated to 70° C. After 12 h, the reaction mixture was quenched by the addition of water and extracted with EtOAc (2×25 mL). Of note, the nitro group was reduced to the aniline during the carbonylation. The organic layer was washed with water, brine, dried over sodium sulfate, filtered, concentrated under reduced pressure and purified by normal phase chromatography by eluting with hexanes/EtOAc to give methyl 3-amino-5-oxo-5,6,7,8-tetrahydronaphthalene-2-carboxylate 8-4 (150 mg, 37% yield). ¹H NMR (500 MHz, CHLOROFORM-d) δ 7.78 (s, 1H), 7.32 (s, 1H), 5.64 (br s, 2H), 3.91 (s, 3H), 2.86 (t, J=6.0 Hz, 2H), 2.66-2.63 (m, 2H), 2.13-2.08 (m, 2H). LC-MS: RT=0.81 min; MS (EST) m/z=220.0 (M+H)⁺; [Method A].

Intermediate 8-5: Me₃Al (0.479 ml, 0.960 mmol) was added to 4-fluoro-3-(trifluoromethyl)aniline (0.172 g, 0.958 mmol) in toluene (2 mL) at 0° C. After 15 min, the Me₃Al mixture was transferred to methyl 3-amino-5-oxo-5,6,7,8-tetrahydronaphthalene-2-carboxylate (Intermediate 8-4) (0.070 g, 0.319 mmol) in toluene (3 mL) and heated to 120° C. under microwave irradiation for 30 min. The reaction mixture was quenched by the addition of 1.0M HCl and extracted with EtOAc (2×25 ml). The organic portion was dried over sodium sulfate, filtered, concentrated under reduced pressure, and purified by normal phase chromatography by eluting with hexanes/EtOAc to give intermediate 8-5 (41 mg, 37% yield). LC-MS: RT=0.96 min; MS (ESI) m/z=367.0 (M+H)⁺; [Method A].

Intermediate 9-4: 4-amino-N-(4-fluoro-3-(trifluoromethyl)phenyl)bicyclo[4.2.0]octa-1,3,5-triene-3-carboxamide. The titled compound was prepared following the method outlined in the scheme below.

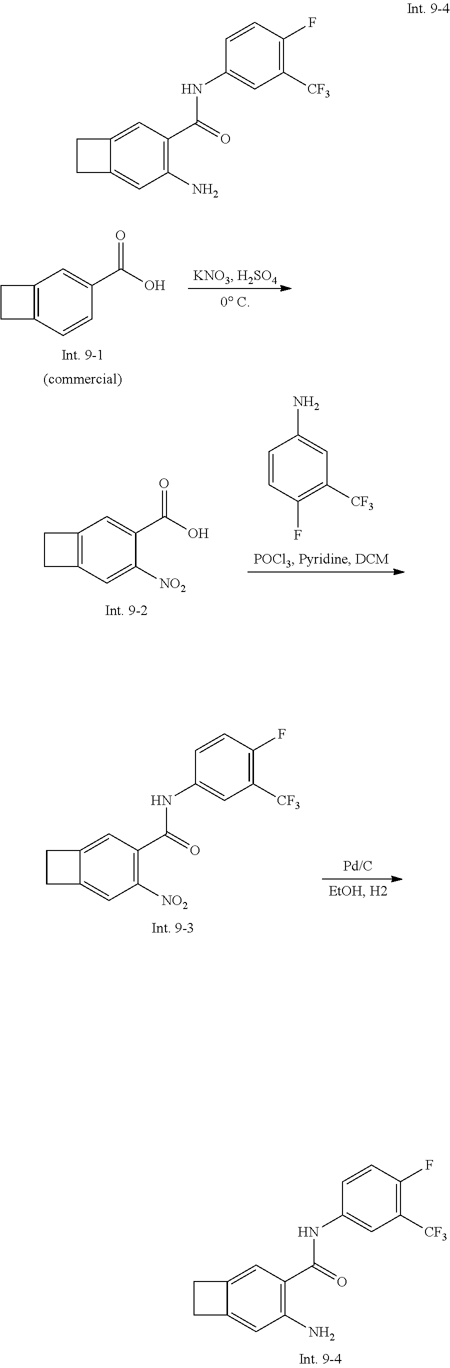 [SMILES: Nc1cc2c(cc1C(=O)Nc1ccc(F)c(C(F)(F)F)c1)CC2 ; O=C(O)c1ccc2c(c1)CC2 ; O=C(O)c1cc2c(cc1[N+](=O)[O-])CC2 ; Nc1ccc(F)c(C(F)(F)F)c1 ; O=C(Nc1ccc(F)c(C(F)(F)F)c1)c1cc2c(cc1[N+](=O)[O-])CC2 ; Nc1cc2c(cc1C(=O)Nc1ccc(F)c(C(F)(F)F)c1)CC2]

Intermediate 9-2: A solution of bicyclo[4.2.0]octa-1(6),2,4-triene-3-carboxylic acid (500 mg, 3.37 mmol) 9-1 in H₂SO₄ (3.0 mL, 56 mmol) at 0° C. was treated with KNO₃ (409 mg, 4.05 mmol) in H₂SO₄ (3.0 mL, 56 mmol). After 3 h, the reaction mixture was quenched with ice and extracted with DCM (2×25 mL). The organic layer was washed with water, brine, dried over sodium sulfate, filtered, and concentrated under reduced pressure to give 9-2 (216 mg, 33% yield) and carried forward to the next reaction without further purification. The regio-selectivity of nitration was confirmed after the next reaction step. LC-MS: RT=0.67 min; MS (ESI) m/z=194.1 (M+H)⁺; [Method A].

Intermediate 9-3: POCl₃ (167 mg, 1.09 mmol) in DCM (1 mL) was added to a solution of 4-nitrobicyclo[4.2.0]octa-1(6),2,4-triene-3-carboxylic acid (Intermediate 9-2) (210 mg, 1.09 mmol) and 4-fluoro-3-(trifluoromethyl)aniline (195 mg, 1.09 mmol) with pyridine (1 mL) DCM (5 mL) at 0° C. The reaction mixture was stirred cold for 1 h then quenched with 1M HCl and extracted with EtOAc (2×50 mL). The organic portion was dried (MgSO₄) and filtered. The residue was partitioned between EtOAc and water, The organic layer was washed with 1M HCl, water, brine, dried over sodium sulfate, filtered, and concentrated under reduced pressure. The residue was purified by normal phase chromatography by eluting with hexanes/EtOAc to give N-(4-fluoro-3-(trifluoromethyl)phenyl)-4-nitrobicyclo[4.2.0]octa-1(6),2,4-triene-3-carboxamide 9-3 (75 mg, 20% yield). ¹H NMR (600 MHz, DMSO-d6) δ 10.95 (s, 1H), 8.15 (dd, J=6.4, 2.6 Hz, 1H), 7.90 (dt, J=8.4, 3.7 Hz, 1H), 7.87 (s, 1H), 7.53 (t, J=9.5 Hz, 1H), 7.52 (s, 1H), 3.29 (s, 4H). LC-MS: RT=0.98 min; MS (ESI) m/z=354.9 (M+H)⁺; [Method A].

Intermediate 9-4: N-(4-fluoro-3-(trifluoromethyl)phenyl)-4-nitrobicyclo[4.2.0]octa-1(6),2,4-triene-3-carboxamide 9-3 (70 mg, 0.20 mmol) was dissolved in MeOH (5 mL) followed by Pd/C (10%, 0.1 g) and hydrogenated at 55 psi. After 2 h, the reaction mixture was filtered through a Celite® pad and concentrated under reduced pressure to give 4-amino-N-(4-fluoro-3-(trifluoromethyl)phenyl)bicyclo[4.2.0]octa-1(6),2,4-triene-3-carboxamide 9-4 (53 mg, 82% yield) as a brown oil. Intermediate 9-4 was used without further purification. LC-MS: RT=0.91 min; MS (ESI) m/z=324.9 (M+H)+; [Method A].

Intermediate 10-4: 6-amino-N-(4-fluoro-3-(trifluoromethyl)phenyl)-2,3-dihydro-1H-indene-5-carboxamide was prepared following the method outlined in the scheme below.

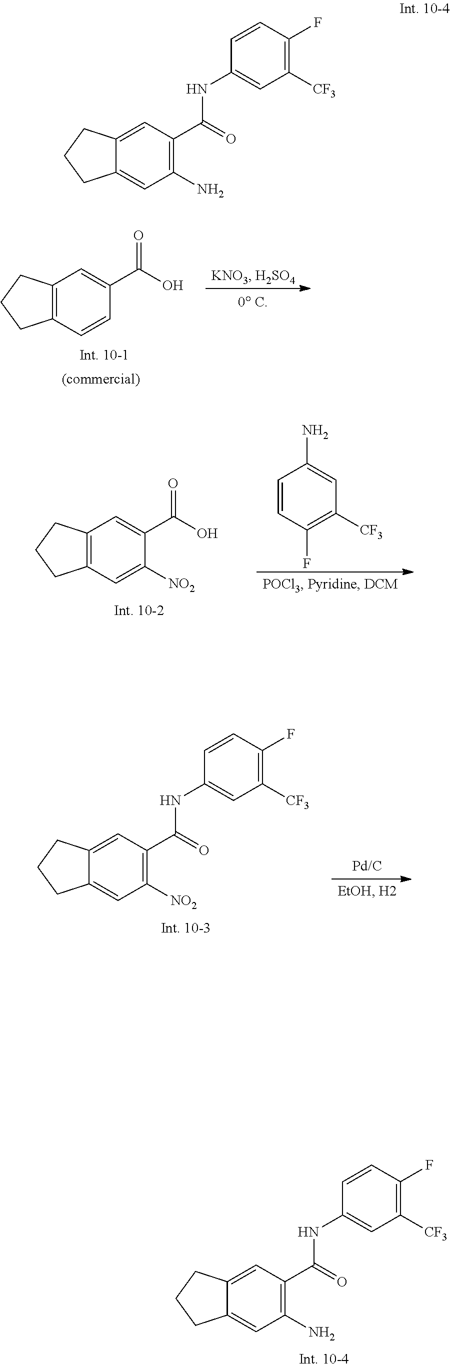 [SMILES: Nc1cc2c(cc1C(=O)Nc1ccc(F)c(C(F)(F)F)c1)CCC2 ; O=C(O)c1ccc2c(c1)CCC2 ; O=C(O)c1cc2c(cc1[N+](=O)[O-])CCC2 ; Nc1ccc(F)c(C(F)(F)F)c1 ; O=C(Nc1ccc(F)c(C(F)(F)F)c1)c1cc2c(cc1[N+](=O)[O-])CCC2 ; Nc1cc2c(cc1C(=O)Nc1ccc(F)c(C(F)(F)F)c1)CCC2]

Intermediate 10-2: A solution of 2,3-dihydro-1H-indene-5-carboxylic acid 10-1 (500 mg, 3.08 mmol) in H₂SO₄ (3.0 mL, 56 mmol) cooled at 0° C. was treated with KNO₃ (374 mg, 3.70 mmol) in H₂SO₄ (3.0 mL, 56 mmol). After 12 h, the reaction mixture was quenched with ice and extracted with DCM. The organic layer was washed with water, brine, dried over sodium sulfate, filtered, and concentrated under reduced pressure to give 10-2 (552 mg, 86% yield). The material was carried forward to the next reaction without further purification. LC-MS RT=0.93 min; MS (ESI) m/z=207.9 (M+H)⁺; [Method A].

Intermediate 10-3: POCl₃ (0.25 mL, 2.66 mmol) in DCM (1 mL) was added to a solution of 6-nitro-2,3-dihydro-1H-indene-5-carboxylic acid (Intermediate 10-2) (552 mg, 2.66 mmol), 4-fluoro-3-(trifluoromethyl)aniline (477 mg, 2.66 mmol), and pyridine (1.7 mL, 21 mmol) in DCM (17.8 mL) at 0° C. After 1 h, the reaction mixture was quenched with 1.0 M HCl, extracted with EtOAc (2×50 mL). The organic portion was dried over sodium sulfate, filtered, concentrated under reduced pressure, and purified by normal phase chromatography by eluting with hexanes/EtOAc to give N-(4-fluoro-3-(trifluoromethyl)phenyl)-6-nitro-2,3-dihydro-1H-indene-5-carboxamide 10-3 (182 mg, 0.490 mmol, 19% yield). ¹H NMR: (400 MHz, DMSO-d6) δ 10.93 (s, 1H), 8.15 (dd, J=6.5, 2.5 Hz, 1H), 8.01 (s, 1H), 7.93-7.88 (m, 1H), 7.63 (s, 1H), 7.56-7.50 (m, 1H), 3.03-2.97 (m, 4H), 2.16-2.09 (m, 2H). LC-MS: RT=1.09 min; MS (ESI) m/z=368.9 (M+H)⁺; [Method A].

Intermediate 10-4: Pd—C (9.82 mg, 0.0920 mmol) was added to a solution of N-(4-fluoro-3-(trifluoromethyl)phenyl)-6-nitro-2,3-dihydro-1H-indene-5-carboxamide (Intermediate 10-3) (170 mg, 0.462 mmol) in EtOH (5 mL) and hydrogenated at 55 psi. After 3 h, the catalyst was filtered through a plug of Celite® and the filtrate concentrated under reduced pressure to give 6-amino-N-(4-fluoro-3-(trifluoromethyl)phenyl)-2,3-dihydro-1H-indene-5-carboxamide (Intermediate 10-4) (136 mg, 87.0% yield). The product was sufficiently pure to carry forward to the next reaction. LC-MS: RT=1.06 min: MS (ESI) m/z=338.9 (M+H)⁺; [Method A].

Intermediate 11-3: 6-amino-N-(4-fluoro-3-(trifluoromethyl)phenyl)-1-oxo-2,3-dihydro-1H-indene-5-carboxamide was prepared following the method outlined in the scheme below.

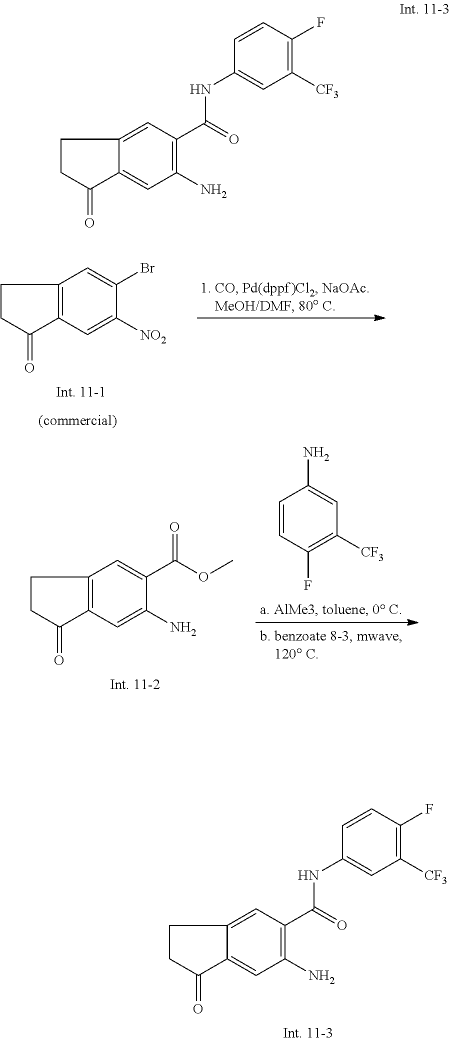

Intermediate 11-2: In a scaled vial 6-amino-5-bromo-2,3-dihydro-1H-inden-1-one (Intermediate 11-1) (0.87 g, 3.9 mmol), TEA (0.54 mL, 3.9 mmol), PdOAc₂ (0.17 g, 0.77 mmol), dppf (0.64 g, 1.2 mmol) were dissolved in a solution of DMSO (12.3 mL) and MeOH (8.2 mL), placed under CO atmosphere (70 psi), scaled and heated to 80° C. for 14 h. The reaction mixture was partitioned with water (50 mL) and ethyl acetate (50 mL). The aqueous layer was extracted with ethyl acetate (2×20 mL), washed with brine (25 mL), dried (MgSO₄), and purified by normal phase chromatography using hexane/ethyl acetate as eluents to give methyl 6-amino-1-oxo-2,3-dihydro-1H-indene-5-carboxylate (Intermediate 11-2) (284 mg, 1.38 mmol, 36.0% yield) as a pale yellow solid. LC-MS: RT=0.74 min: MS (ESI) m/z=206.08 (M+H)⁺; [Method A].

Intermediate 11-3: To 4-fluoro-3-(trifluoromethyl)aniline (262 mg, 1.46 mmol) in toluene (4 mL), cooled to 0° C. was added trimethylaluminum (0.73 mL, 1.5 mmol). After 10 min, methyl 6-amino-1-oxo-2,3-dihydro-1H-indene-5-carboxylate (Intermediate 11-2) (100 mg, 0.49 mmol) in toluene (2 mL) was added, and the resulting mixture heated under microwave irradiation at 120° C. for 30 min. The reaction mixture was quenched with 1M HCl, extracted with EtOAc (2×25 mL), dried over sodium sulfate, concentrated under reduced pressure, and purified by normal phase chromatography using hexane/ethyl acetate as eluents to give 6-amino-N-(4-fluoro-3-(trifluoromethyl)phenyl)-1-oxo-2,3-dihydro-1H-indene-5-carboxamide (Intermediate 11-3) (44 mg, 0.13 mmol, 26% yield) concentrated under reduced pressure to a yellow oil. ¹H NMR (400 MHz, CHLOROFORM-d) δ 8.04-8.01 (m, 1H), 7.89-7.82 (m, 2H), 7.55-7.52 (m, 1H), 7.20-7.17 (m, 1H), 7.12-7.08 (m, 1H), 5.38 (br s, 2H), 3.07-3.01 (m, 2H), 2.72-2.68 (m, 2H). LC-MS: RT=1.21 min; MS (ESI) m/z: =353.3 (M+H)⁺; [Method A].

Intermediate 12-2 (diastereomeric mixture): Preparation of 5-(5-(tert-butoxycarbonyl)-3a,5,6,6a-tetrahydro-4H-pyrrolo[3,4-d]isoxazol-3-yl)-2-methoxybenzoic acid.

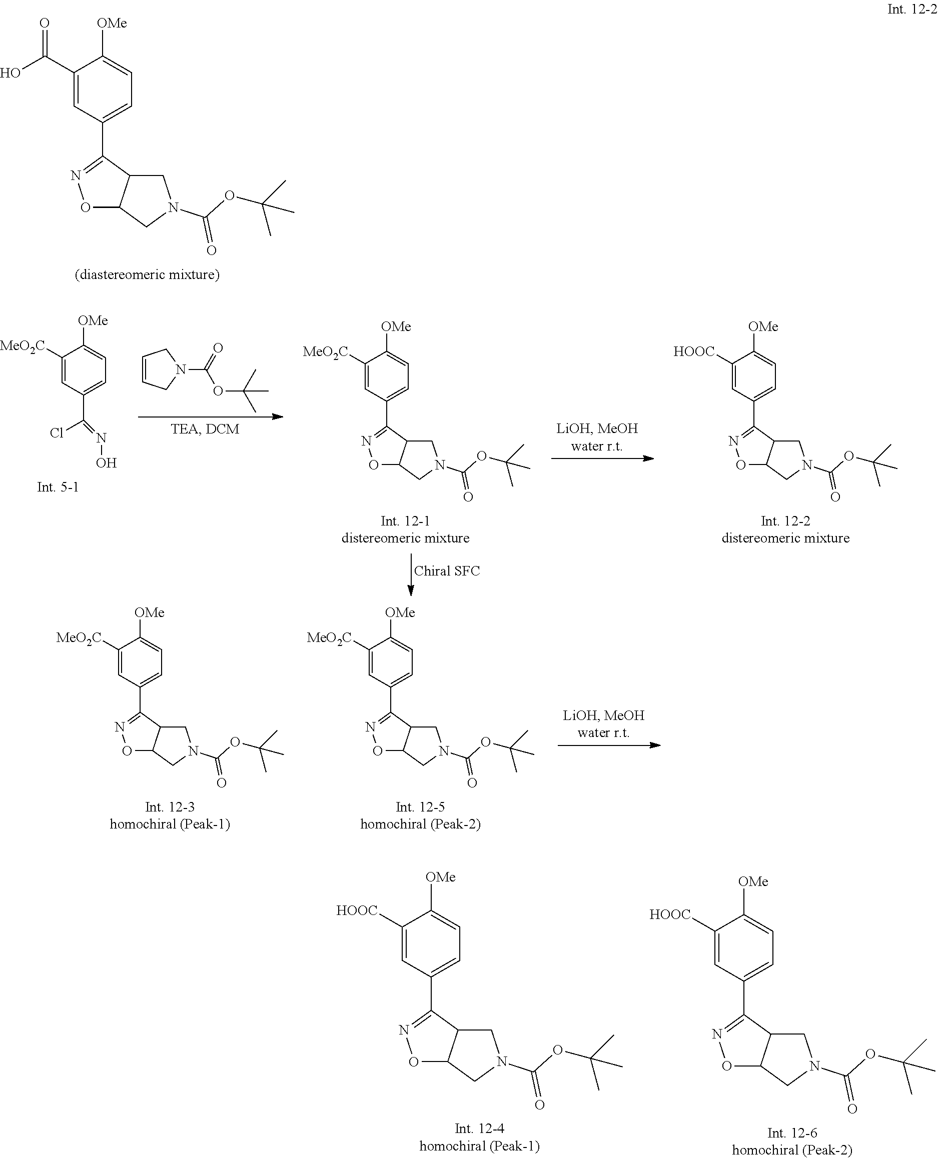

Intermediate 12-1: Preparation of tert-butyl 3-(4-methoxy-3-(methoxycarbonyl)phenyl)-3a,4,6,6a-tetrahydro-5H-pyrrolo[3,4-d]isoxazole-5-carboxylate. Intermediate 12-1 (500 mg, 46% yield) was prepared by the method described for intermediate 6-1 by substituting cyclopent-3-en-1-ylmethanol with tert-butyl 2,5-dihydro-1H-pyrrole-1-carboxylate. ¹H NMR: (400 MHz, CDCl₃) δ 7.99 (d, J=2.4 Hz, 1H), 7.84 (dd, J=8.7, 2.3 Hz, 1H), 7.04 (d, J=8.8 Hz, 1H), 5.31 (ddd, J=9.2, 5.4, 1.2 Hz, 1H), 4.21 (br dd, J=12.4, 9.1 Hz, 1H), 3.96 (s, 3H), 3.91 (s, 3H), 3.72-3.61 (m, 2H), 1.43 (s, 9H). MS (ESI) m/z=377.4 (M+H)⁺; [Method A].

Intermediate 12-2: Preparation of 5-(5-(tert-butoxycarbonyl)-3a,5,6,6a-tetrahydro-4H-pyrrolo[3,4-d]isoxazol-3-yl)-2-methoxybenzoic acid. 12-2 (150 mg, 44% yield) was prepared by the method described for intermediate 6-2 from intermediate 12-1. MS (ESI) m/z=363.4 (M+H)⁺; [Method A].

### Homochiral Intermediates 12-4 & 12-6

Homochiral intermediates 12-3 and 12-5 were obtained by chiral SFC of diastereomeric mixture intermediate 12-1 (499 mg, 1.33 mmol). Chiral SFC Preparative chromatographic conditions: Instrument: Berger MG II (SFC); Column: Regis Whelk-01, 21×250 mm, 5 micron: Mobile phase: 15% MeOH/85% CO₂; Flow conditions: 45 mL/min, 150 Bar, 40° C.; Detector wavelength: 220 nm; Injections details: 1.0 mL of ~31 mg/mL in MeOH-ACN. Analytical chromatographic conditions: Instrument: Shimadzu Nexera SFC; Column: Regis Whelk-01, 4.6×100 mm, 3 micron; Mobile phase: 15% MeOH/85% CO₂; Flow conditions: 2.0 mL/min, 150 Bar, 40° C.: Detector wavelength: 220 nm; Injection details: 5 μL of ~1 mg/mL in Acetonitrile.

Homochiral methylbenzoate intermediate 12-3 (Peak-1, >99% ee, analytical RT=4.02 min) was obtained as a white solid (96 mg, 19% yield). ¹H NMR: (600 MHz, CDCl₃) S 7.99 (d, J=2.3 Hz, 1H), 7.86-7.82 (m, 11H), 7.04 (br d, J=8.7 Hz, 1H), 5.31 (ddd, J=9.2, 5.4, 1.3 Hz, 1H), 4.24-4.18 (m, 1H), 4.01-3.93 (m, 4H), 3.91 (s, 3H), 3.83-3.76 (m, 1H), 3.71-3.67 (m, 1H), 3.63 (br s, 1H), 1.43 (br s, 9H).

Homochiral benzoic acid intermediate 12-4 (Peak-1). Preparation of 5-(5-(tert-butoxycarbonyl)-3a,5,6,6a-tetrahydro-4H-pyrrolo[3,4-d]isoxazol-3-yl)-2-methoxybenzoic acid. Intermediate 12-4 (52 mg, 68% yield) was prepared in a similar manner as intermediate 12-2 with the hydrolysis of intermediate 12-3. MS (EST) m/z=363.1 (M+H)⁺; [Method A].

Homochiral methylbenzoate intermediate 12-5 (Peak-2, 99.6% ee, analytical RT=4.56 min) was obtained as a white solid (96.7 mg, 19.4% yield). 1H NMR (600 MHz, CDCl₃) S 7.98 (d, J=2.3 Hz, 1H), 7.83 (dd, J=8.8, 2.2 Hz, 1H), 7.03 (d, J=8.7 Hz, 11H), 5.32-5.28 (m, 1H), 4.21 (td, J=8.8, 4.0 Hz, 1H), 4.01-3.94 (m, 1H), 3.95 (s, 3H), 3.90 (s, 3H), 3.83-3.73 (m, 1H), 3.68 (dd, J=11.4, 8.9 Hz, 1H), 3.65-3.58 (m, 1H), 1.43 (s, 9H).

Homochiral benzoic acid intermediate 12-6 (Peak-2). Preparation of 5-(5-(tert-butoxycarbonyl)-3a,5,6,6a-tetrahydro-4H-pyrrolo[3,4-d]isoxazol-3-yl)-2-methoxybenzoic acid. Intermediate 12-6 (48 mg, 62.3% yield) was prepared in a similar manner as intermediate 12-2 with the hydrolysis of intermediate 12-5. MS (EST) m/z=363.1 (M+H)⁺; [Method A].

Intermediate 13-2: tert-butyl 2-amino-2-(6-fluoro-3′-((3-((4-fluoro-3-(trifluoromethyl)phenyl)-carbamoyl)-5,6,7,8-tetrahydronaphthalen-2-yl)carbamoyl)-4′-methoxy-[1,1′-biphenyl]-3-yl)acetate was prepared following the method outlined in the scheme below.

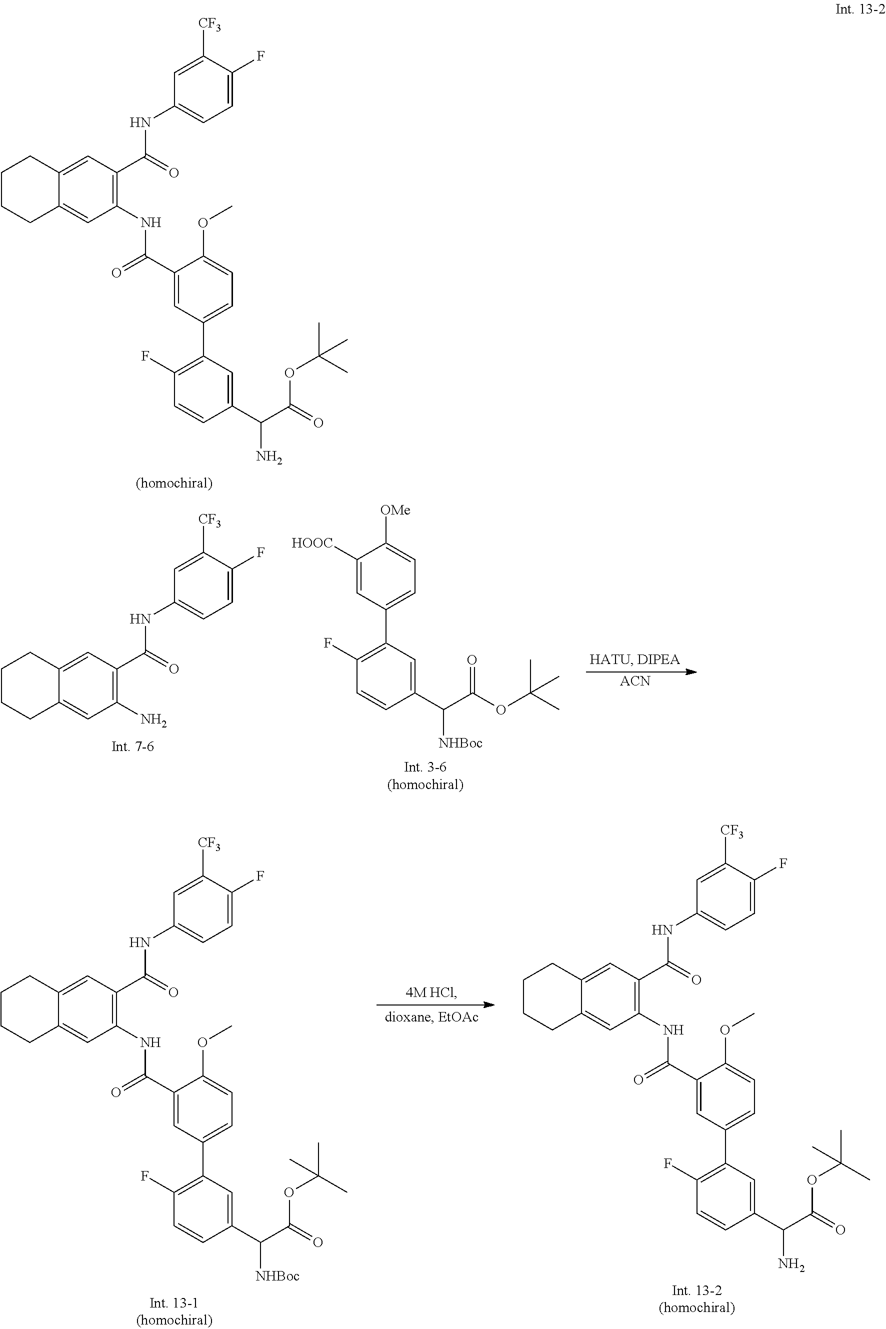

Intermediate 13-1: Intermediate 7-6 (40 mg, 0.11 mmol) was added to ACN (4.54 mL) followed by DIPEA (0.46 mL, 2.1 mmol), 5′-(2-(tert-butoxy)-1-((tert-butoxycarbonyl)amino)-2-oxoethyl)-2′-fluoro-4-methoxy-[1,1′-biphenyl]-3-carboxylic acid (Intermediate 3-6) (54.0 mg, 0.110 mmol) and HATU (51.8 mg, 0.140 mmol). After stirring for 12 h, the reaction mixture was concentrated under reduced pressure and purified directly by reverse phase chromatography HPLC (using gradients of mobile phase A: 20% ACN-80% H₂O-0.1% TFA; mobile phase B: 80% ACN-20% H₂O-0.1% TFA) to give a solid. ¹H NMR (400 MHz, CHLOROFORM-d) δ 11.65-11.61 (m, 1H), 9.25 (br s, 1H), 8.58-8.54 (m, 1H), 8.49-8.45 (m, 1H), 8.07 (s, 1H), 7.97-7.89 (m, 1H), 7.72-7.68 (m, 1H), 7.49 (dd, J=7.3, 2.2 Hz, 1H), 7.35-7.31 (m, 1H), 7.18-7.11 (m, 3H), 5.65 (br d, J=6.6 Hz, 1H), 5.25 (br d, J=7.0 Hz, 1H), 4.17 (s, 3H), 2.44-2.33 (m, 4H), 1.62-1.55 (m, 4H), 1.47-1.39 (m, 18H). LC-MS: RT=1.39 min; MS (ESI) m/z=810.2 (M+H)⁺; [Method A].

Intermediate 13-2: The BOC group was removed from Intermediate 13-1 by dissolving the residue in EtOAc (5 mL) followed by treatment with HCl (4.0 M in dioxane) (2 mL, 8.0 mmol). After stirring for 3 h, the reaction mixture was concentrated under reduced pressure to afford intermediate 13-2 which was used without further purification.

Analytical LC-MS: RT=1.14 min; MS (ESI) m/z=710.2 (M+H)⁺; HPLC purity 93%: [Method A].

Homochiral intermediate 14-1: (S)-1-(3-bromo-4-fluorophenyl)-2,2,2-trifluoroethan-1-ol was prepared following the method outlined in the scheme below.

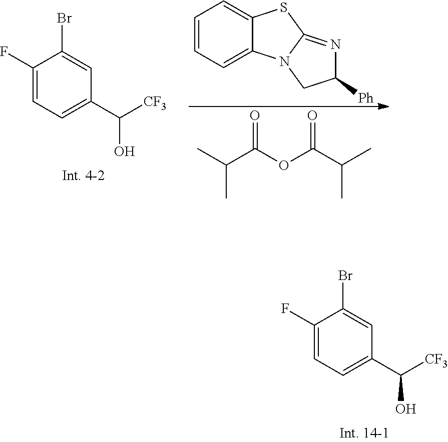

A solution of (S)-2-phenyl-2,3-dihydrobenzo[d]imidazo[2,1-b]thiazole (0.163 g, 0.645 mmol) and (racemic intermediate 4-2) (4.4 g, 16.12 mmol) in diisopropyl ether (54 mL) was chilled between 0° C. to −20° C. The solution was treated with isobutyric anhydride (1.6 mL, 9.67 mmol) and transferred to a freezer for 14 h. The reaction mixture was quenched by the addition of MeOH (~3 mL), extracted from phosphate buffer with EtOAc (2×25 mL). The organic portion was concentrated under reduced pressure and purified by normal phase chromatography using hexane/ethyl acetate as eluents to afford (S)-1-(3-bromo-4-fluorophenyl)-2,2,2-trifluoroethan-1-ol (chiral intermediate 14-1) (1.7 g, 39% yield, 99.9% ee) ¹H NMR (500 MHz, CHLOROFORM-d) δ 7.75-7.71 (m, 1H), 7.44-7.39 (m, 1H), 7.19-7.13 (m, 1H), 5.01 (q, J=6.6 Hz, 1H), 4.15-4.10 (m, 1H).

Intermediate 15-1: Preparation of (3-((3-((4-fluoro-3-(trifluoromethyl)phenyl)carbamoyl)-5,6,7,8-tetrahydronaphthalen-2-yl)carbamoyl)-4-methoxyphenyl)boronic acid

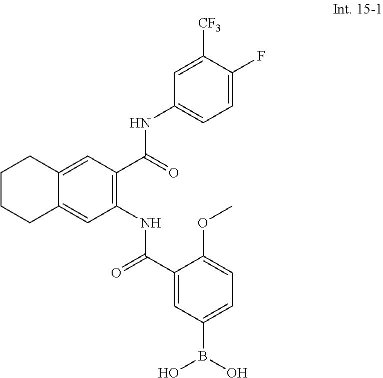 [SMILES: COc1ccc(B(O)O)cc1C(=O)Nc1cc2c(cc1C(=O)Nc1ccc(F)c(C(F)(F)F)c1)CCCC2]

Intermediate 7-6 (100 mg, 0.28 mmol) was added to ACN (11.4 mL) followed by DIPEA (1.14 mL, 6.53 mmol) and 2-methoxy-5-(4,4,5,5-tetramethyl-1,3,2-dioxaborolan-2-yl)benzoic acid (79 mg, 0.28 mmol) and HATU (130 mg, 0.34 mmol). After 4 h, the reaction mixture was extracted from water with EtOAc, the organic portion washed with brine, dried over sodium sulfate, filtered, concentrated under reduced pressure, and purified by reverse phase chromatography (using gradients of mobile phase A: 20% ACN-80% H₂O-0.1% TFA: mobile B: 80% ACN-20% H₂O-0.1% TFA) to give intermediate 15-1 (51.4 mg, 34% yield). ¹H NMR (400 MHz, DMSO-d6) δ 11.51-11.49 (m, 1H), 10.75 (s, 1H), 8.49-8.45 (m, 1H), 8.37-8.30 (m, 2H), 8.09-8.02 (m, 2H), 7.95 (dd, J=8.4, 2.0 Hz, 1H), 7.57-7.51 (m, 3H), 7.16 (d, J=8.4 Hz, 1H), 3.99 (s, 3H), 2.79 (br d, J=9.2 Hz, 4H), 1.79 (br d, J=2.4 Hz, 4H). Analytical LC-MS: RT=1.22 min, MS (ESI) m/z=530.8 (M+H)⁺; [Method A].

Intermediate 16-1: Preparation of 3-bromo-N-(cyclobutylmethyl)-4-fluorobenzamide.

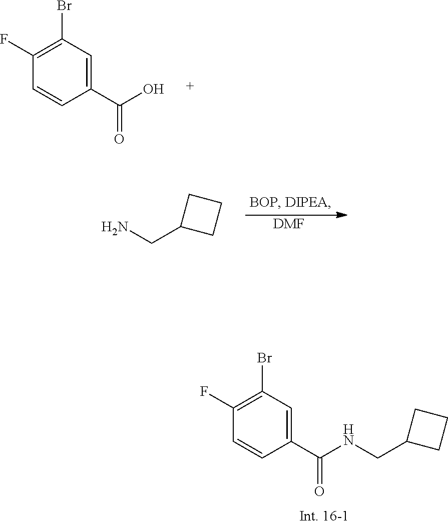

BOP (202 mg, 0.460 mmol) was added to a solution of 3-bromo-4-fluorobenzoic acid (100 mg, 0.46 mmol), cyclobutylmethanamine (58.3 mg, 0.690 mmol), and DIPEA (0.16 mL, 0.91 mmol) in DMF (3 mL). After 4 h, the reaction mixture was purified directly by reverse phase chromatography HPLC using gradients of mobile phase A: 20% ACN −80% H₂O-0.1% TFA; mobile phase B: 80% ACN-20% H₂O-0.1% TFA to give intermediate 16-1 (32 mg, 25% yield) as a solid. Analytical LC-MS: RT=1.18 min: MS (ESI) m/z=286.1 (M+H)+; [Method A].

Intermediate 17-3: 5-(3-hydroxypropyl)-2-methoxybenzoic acid was prepared following the method outlined in the scheme below.

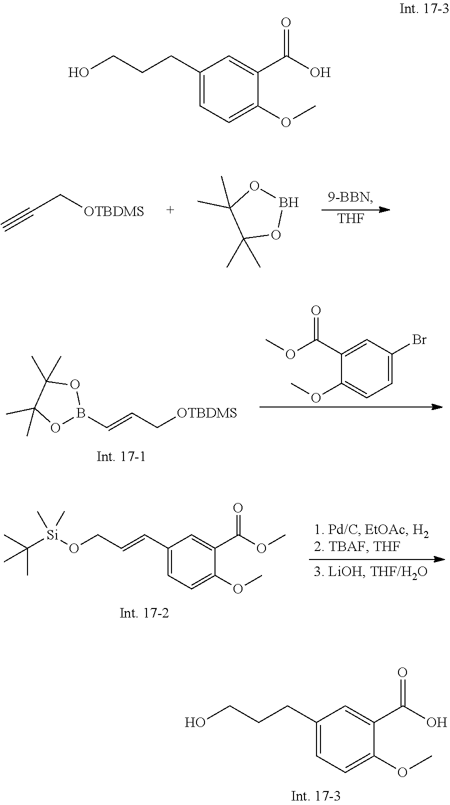

Intermediate 17-1: To tert-butyldimethyl(prop-2-ynyloxy)silane (2 g, 11.74 mmol) was added THF (8 mL), 4,4,5,5-tetramethyl-1,3,2-dioxaborolane (3.07 mL, 21.14 mmol) and 9-BBN, 0.5 N in THF (2.35 mL, 1.17 mmol) and stirred at 75° C. for 14 h. The reaction was carefully quenched with water (gas evolution), stirred at rt for 1 h, diluted with EtOAc (50 mL). The organic layer was separated, washed with brine, dried over MgSO₄, filtered, concentrated under reduced pressure, and purified by normal phase chromatography using hexane/ethyl acetate as eluents to give intermediate 17-1 (1.9 g, 53% yield) as a clear oil. ¹H NMR (400 MHz, CHLOROFORM-d) δ ppm 6.68 (1H, dt, J=17.9, 3.4 Hz), 5.75 (1H, d, J=17.9 Hz), 4.22-4.27 (2H, m), 1.27 (12H, s), 0.92 (9H, s), 0.07 (6H, s).

Intermediate 17-2: A mixture of intermediate 17-1 (0.84 g, 2.8 mmol) and methyl 5-bromo-2-methoxybenzoate (0.66 g, 2.7 mmol) in DMF (6 mL) were degassed with N₂, followed by the addition of XPhosPdG2 (0.106 g, 0.130 mmol). The reaction vessel was sealed and heated to 60° C. After 1.5 h, the cooled reaction mixture was diluted with EtOAc (50 mL), separated and washed with H₂O, brine, dried over sodium sulfate, filtered, concentrated under reduced pressure, and purified by normal phase chromatography using hexane/ethyl acetate as eluents to give intermediate 17-2 (752 mg, 83% yield). ¹H NMR (500 MHz, CDCl₃) δ 7.83 (d, J=2.3 Hz, 1H), 7.50 (dd, J=8.7, 2.3 Hz, 11H), 6.95 (d, J=8.7 Hz, 1H), 6.55 (dt, J=15.8, 1.6 Hz, 1H), 6.24 (t, J=5.0 Hz, 1H), 6.21 (t, J=5.0 Hz, 1H), 4.36 (dd, J=5.0, 1.7 Hz, 2H), 3.93 (s, 3H), 3.92 (s, 3H), 0.96 (s, 9H), 0.14-0.13 (m, 6H).

Intermediate 17-3: Methyl (E)-5-(3-((tert-butyldimethyl-silyl)oxy)prop-1-en-1-yl)-2-methoxybenzoate (Intermediate 17-2, 693 mg, 2.06 mmol) was dissolved in EtOAc (15 mL) and was hydrogenated at 55 psi for 3 h. The suspension was filtered through a plug of Celite® and the filtrate concentrated under reduced pressure. The residue was taken up in THF (20 mL), cooled to 0° C., and TBAF (2.059 mL, 2.06 mmol) was added. After 2 h, the reaction mixture was treated with water (20 mL), extracted with ethyl acetate (2×20 mL), and the combined organic portions washed with brine (15 mL), and concentrated under reduced pressure. The benzoate was hydrolyzed by dissolution in a solution in THF/MeOH (1:1, 10 mL) and LiOH (3.09 mL, 6.18 mmol) was added. After stirring for 14 h. the reaction was quenched with dilute HCl (1 N, 20 mL) and extracted with ethyl acetate (3×30 mL). The combined organic portion was washed with brine (15 mL), dried (MgSO₄), filtered, and concentrated under reduced pressure to give intermediate 17-3 (0.5 g, 115% yield) yellow oil. ¹H NMR (500 MHz, CDCl₃) δ 8.05 (d, J=2.3 Hz, 1H), 7.44 (dd, J=8.5, 2.4 Hz, 1H), 7.01 (d, J=8.4 Hz, 1H), 5.32 (s, 1H), 4.12-4.06 (m, 4H), 3.69 (t, J=6.3 Hz, 2H), 2.81-2.71 (m, 2H), 1.96-1.87 (m, 2H). Analytical LC-MS: RT=0.85 min; MS (ESI) m/z=211.2 (M+H); [Method A].

Intermediate 18-3: 5-(3-hydroxy-3-methylbutyl)-2-methoxybenzoic acid was prepared following the method outlined in the scheme below.

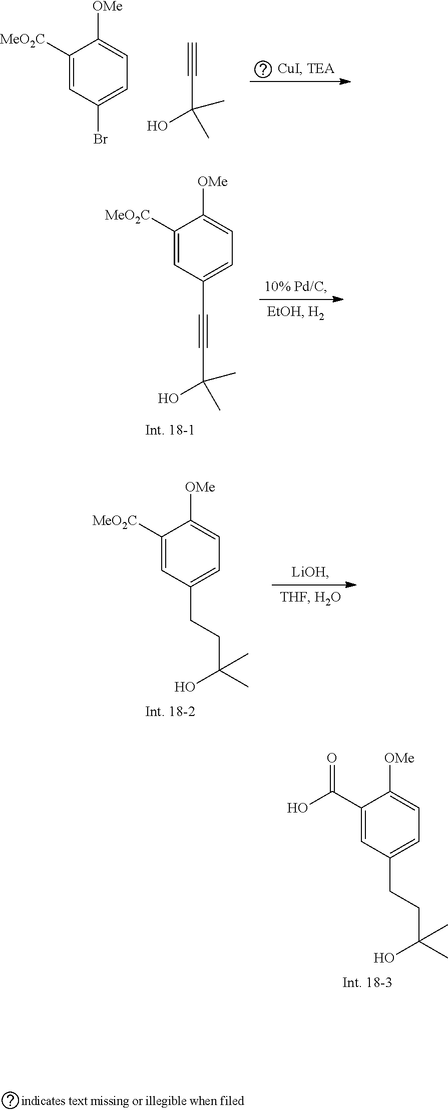

Intermediate 18-1: In a sealed vial methyl 5-bromo-2-methoxybenzoate (1.7 g, 6.94 mmol), 2-methylbut-3-yn-2-ol (0.584 g, 6.94 mmol), Pd(Ph₃P)₄ (0.401 g, 0.35 mmol), copper(I) iodide (0.013 g, 0.069 mmol) was added followed by TEA (15 mL). The reaction mixture was degassed, sealed, and heated at 80° C. The reaction was diluted with water (20 mL), extracted with ethyl acetate (50 mL), the organic portion was washed with brine (15 mL), dried (MgSO₄), filtered, concentrated under reduced pressure, and purified by normal phase chromatography using hexane/ethyl acetate as eluents to give intermediate 18-1 (1.2 g, 70% yield) as a yellow oil. ¹H NMR (400 MHz, CHLOROFORM-d) δ 7.88 (d, J=2.2 Hz, 1H), 7.52 (dd, J=8.8, 2.2 Hz, 1H), 6.89 (s, 1H), 3.92 (s, 3H), 3.90 (s, 3H), 1.62 (s, 6H). MS (ESI) m/z=249 (M+H)⁺. Intermediate 18-2: Methyl 5-(3-hydroxy-3-methylbut-1-yn-1-yl)-2-methoxybenzoate (1.2 g, 4.8 mmol) was dissolved in EtOH (25 mL) and to this solution was added wet 10% Pd/C (0.2 g) and the reaction mixture hydrogenated at 20 psi for 14 h. The suspension was filtered through a plug of Celite®, concentrated under reduced pressure, and purified by normal phase chromatography using hexane/ethyl acetate as eluents to give intermediate 18-2 (714 mg, 59.0% yield) as a pale yellow oil. ¹H NMR (400 MHz, CDCl₃) δ 7.67 (d, J=2.4 Hz, 1H), 7.33 (dd, J=8.4, 2.4 Hz, 1H), 6.94 (d, J=8.4 Hz, 1H), 3.92 (s, 3H), 3.92 (s, 3H), 2.88-2.59 (m, 2H), 1.86-1.69 (m, 2H), 1.32 (s, 6H), 1.25 (s, 1H). MS (ESI) m/z=253.3 (M+H)⁺.

Intermediate 18-3: To solution of intermediate 18-2 (0.714 g, 2.83 mmol) was dissolved in THF (21 mL)/Water (7 mL) and LiOH (1.4 mL, 2.83 mmol) was added. After stirring for 12 h, the reaction mixture was diluted with 0.1 N HCl, extracted with EtOAc (2×25 mL). The combined organic portions were washed with brine, dried (MgSO₄), filtered, and concentrated to give intermediate 18-3 (0.60 g, 89% yield) as a solid. Analytical LC-MS: RT=0.95 min: MS (EST) m/z=239.2 (M+H)⁺; Method A.

Intermediate 19-2: 5-(4,5-bis(2-hydroxyethyl)isoxazol-3-yl)-2-methoxybenzoic acid was prepared following the method outlined in the scheme below.

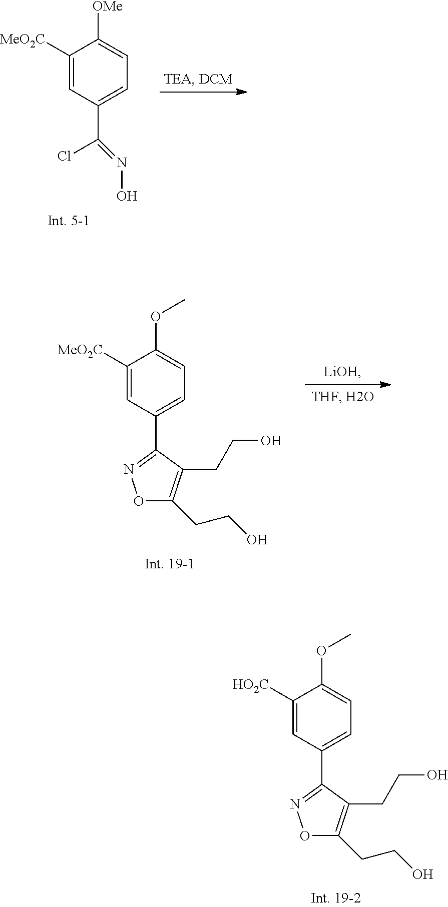 [SMILES: COC(=O)c1cc(/C(Cl)=N/O)ccc1OC ; COC(=O)c1cc(-c2noc(CCO)c2CCO)ccc1OC ; COc1ccc(-c2noc(CCO)c2CCO)cc1C(=O)O]

Intermediate 19-1: Intermediate 5-1 (1.0 g, 4.10 mmol) was dissolved in DCM (41 mL) and treated with hex-3-yne-1,6-diol (937 mg, 8.21 mmol) followed by TEA (1.7 mL, 12.31 mmol) at rt After 12 h, the reaction mixture was concentrated under reduced pressure and purified by normal phase chromatography using hexane/ethyl acetate as eluents to give intermediate 19-1. 1H NMR (500 MHz, DMSO-d6) δ 8.10 (d, J=2.3 Hz, 1H), 8.00 (dd, J=8.7, 2.3 Hz, 11-1), 7.32-7.28 (m, 1H), 4.63-4.59 (m, 1H), 3.89 (s, 3H), 3.82 (s, 3H), 3.76 (t, J=6.5 Hz, 2H), 3.53-3.46 (m, 4H), 3.04 (t, J=6.4 Hz, 2H). Analytical LC-MS: RT=1.01 min; MS (ESI) m/z=322.2 (M+H)⁺; [Method A]. Intermediate 19-2: The ester intermediate 19-1 was dissolved in MeOH/THF (1:1, 20 mL) and treated with LiOH monohydrate (517 mg, 12.3 mmol) dissolved in H₂O (3 mL). After 3 h, the reaction mixture was concentrated under reduced pressure, the remaining aqueous layer was acidified with 1.0 M HCl solution, extracted with EtOAc (2×25 mL). The organic portion was washed with H₂O, brine, dried over sodium sulfate, filtered, and concentrated under reduced pressure to give 5-(4,5-bis(2-hydroxyethyl)isoxazol-3-yl)-2-methoxybenzoic acid (640 mg, 51% yield) as a solid. Analytical LC-MS: RT=0.91 min; MS (ESI) m/z=308.2 (M+H)⁺; [Method A].

Intermediate 20-2: (S)-5′-(I-((cyclobutylcarbamoyl)oxy)-2,2,2-trifluoroethyl)-2′-fluoro-4-methoxy-[1,1′-biphenyl]-3-carboxylic acid was prepared following the method outlined in the scheme below.

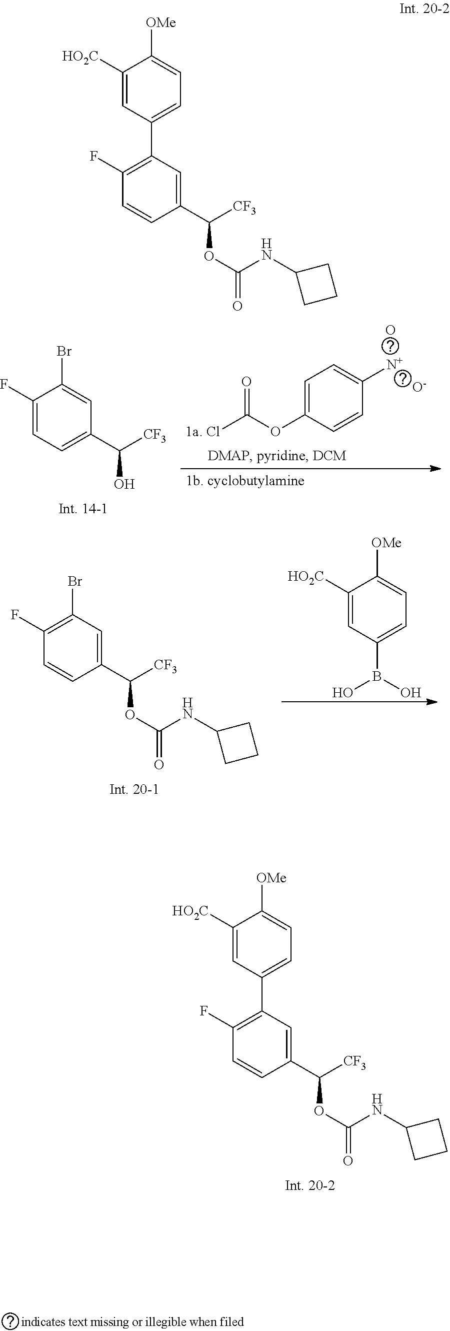

Intermediate 20-1: Preparation of(S)-1-(3-bromo-4-fluorophenyl)-2,2,2-trifluoroethyl cyclobutylcarbamate. A mixture of intermediate 14-1 (300 mg, 1.10 mmol), pyridine (0.44 mL, 5.49 mmol), and DMAP (13.42 mg, 0.11 mmol) was dissolved in DCM (20 mL) and 4-nitrophenyl carbonochloridate (1.1 g, 5.49 mmol) was added. The reaction mixture was allowed to stir for 1 h followed by the addition of cyclobutanamine (0.78 g, 10.99 mmol). After 2 h. the reaction was concentrated and purified by normal phase chromatography using hexan/ethyl acetate as eluents to afford intermediate 20-1 (347.5 mg, 0.94 mmol, 85% yield) as a white solid. ¹H NMR (400 MHz, CHLOROFORM-d) δ 7.68-7.64 (m, 1H), 7.40-7.35 (m, 1H), 7.18-7.13 (m, 1H), 6.02-5.95 (m, 1H), 4.19-4.09 (m, 1H), 2.41-2.28 (m, 2H), 1.97-1.84 (m, 2H), 1.77-1.63 (m, 2H), 1.55 (s, 1H).

Intermediate 20-2: Into a reaction vessel containing intermediate 20-1 (347 mg, 0.800 mmol) was added 5-borono-2-methoxybenzoic acid (203 mg, 1.04 mmol), PdCl₂(dppf)-CH₂Cl₂Adduct (98 mg, 0.12 mmol), Na₂CO₁ (338 mg, 3.19 mmol), THF (11.5 mL) and H₂O (3 mL). The reaction mixture was degassed by bubbling N₂ for 10 min, scaled, and stirred at 65° C. for 3 h. After cooling to rt, the reaction was quenched with 1N HCl, extracted with EtOAc (2×25 mL), the organic portions were dried over Na₂SO₄, concentrated under reduced pressure, purified by reverse phase chromatography, and lyophilized to afford intermediate 20-2 (72 mg, 21% yield). Analytical LC-MS: RT=0.94 min; MS (ESI) m/z=442.0 (M+H)⁺; [Method A].

Intermediate 21-2: 2-(4-bromo-1H-pyrazol-1-yl)acetic acid was prepared following the method outlined in the scheme below.

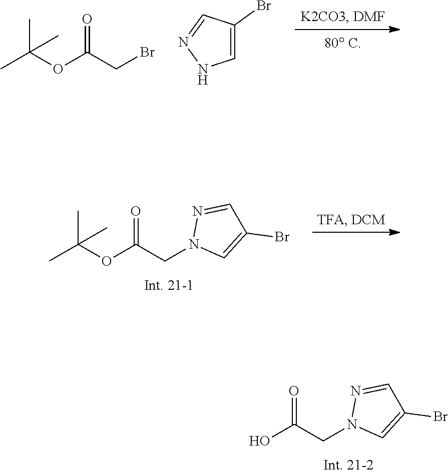

Intermediate 21-1: K₂CO₃ (2.82 g, 20.4 mmol) was added to a solution of 4-bromo-1H-pyrazole (1 g, 7 mmol) in DMF (27.2 ml) at 80° C. After 5 min, tert-butyl 2-bromoacetate (1.99 g, 10.2 mmol) was added and the reaction mixture stirred for 14 h before quenching water and extracted with DCM (2×25 mL). The organic layer was washed with water, brine, dried over sodium sulfate, filtered, concentrated under reduced pressure, and purified by normal phase chromatography to afford intermediate 21-1 (1.78 g, 6.81 mmol, 100% yield) as a clear, colorless oil. ¹H NMR (500 MHz, CHLOROFORM-d) δ 7.51-7.49 (m, 2H), 4.78 (s, 2H), 1.48 (s, 9H).

Intermediate 21-2: TFA (2.2 mL, 28.7 mmol) was added to a solution of intermediate 21-1 (500 mg, 1.91 mmol) in DCM (7.66 mL). After 2 h, the reaction mixture was concentrated in vacuo to dryness. The residue was dissolved in EtOAc (20 mL), neutralized with NaHCO₃ solution, re-acidified with 1.0 M HCl solution and extracted with EtOAc (2×25 mL). The organic portion was dried over sodium sulfate, filtered, and concentrated under reduced pressure to generate intermediate 21-2 (323 mg, 82%) which was used without further purification. ¹H NMR (500 MHz, CHLOROFORM-d) δ 7.51-7.49 (m, 2H), 4.78 (s, 2H), 1.48 (s, 9H). Analytical LC-MS: RT=0.81 min; MS (ESI) m/z=205.0 (M+H)⁺; [Method A].

Intermediate 22-2: 5-(1,1-dioxidoisothiazolidin-2-yl)-2-methoxybenzoic acid.

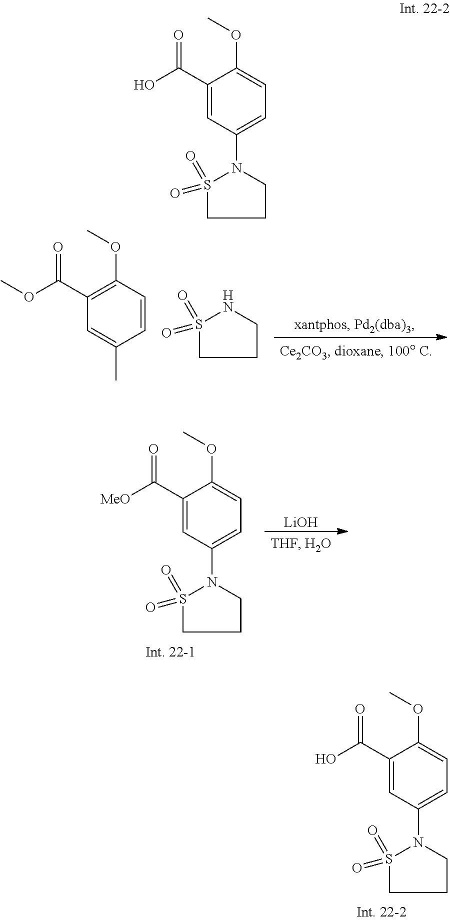

Intermediate 22-1: To a solution containing isothiazolidine 1,1-dioxide (41.5 mg, 0.340 mmol), in dioxane (1.8 mL) was added methyl 5-iodo-2-methoxybenzoate (100 mg, 0.342 mmol), Xantphos (20 mg, 0.034 mmol), cesium carbonate (223 mg, 0.685 mmol), and the reaction mixture was purged with nitrogen for 10 min followed by addition of Pd₂(dba)₃ (16 mg, 0.017 mmol). The reaction vessel was sealed and heated at 100° C. for 15 h. The reaction mixture was partitioned with water (10 mL) and ethyl acetate (30 mL). The aqueous layer was extracted with ethyl acetate (2×20 mL). The combined organic layers were washed with brine (15 mL), dried over MgSO₄, filtered, and concentrated under reduced pressure. Analytical LC-MS: RT=0.93 min; MS (ESI) m/z=286.1 (M+H)⁺; [Method A].

Intermediate 22-2: Benzoate intermediate 22-1 was dissolved in THF/MeOH/water (20 mL), cooled to 0° C., and LiOH solution (0.171 mL, 0.342 mmol) was added. After 3 h, the reaction mixture was partitioned between water (10 mL) and Et₂O (50 mL). The aqueous layer was acidified with 1 N HCl solution, extracted with EtOAc (3×20 mL) and the organic extract washed with brine (15 mL) and dried over MgSO₄, filtered, and concentrated under reduced pressure to give 5-(1,1-dioxidoisothiazolidin-2-yl)-2-methoxybenzoic acid (70 mg, 75% yield) as a brown oil. Analytical LC-MS: RT=0.83 min; MS (ESI) m/z=272.1 (M+H)⁺; [Method A].

Intermediate 23-1: Preparation of 2-(3-bromo-4-fluorophenyl)-N-(cyclobutylmethyl)acetamide.

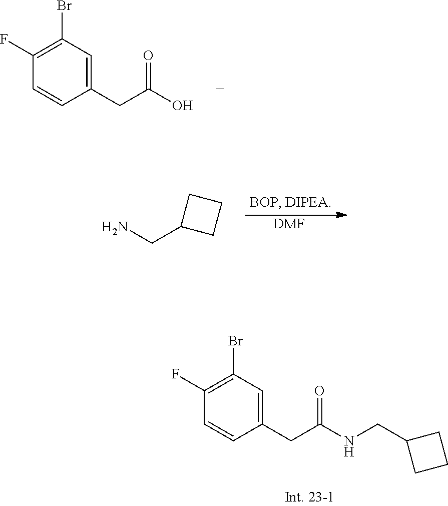

Intermediate 23-1 (44 mg, 34%) was prepared in a similar manner as Intermediate 16-1 replacing 3-bromo-4-fluorobenzoic acid with 2-(3-bromo-4-fluorophenyl)acetic acid (100 mg, 0.429 mmol)

### Example 1

### 6-fluoro-3′-((3-((4-fluoro-3-(trifluoromethyl)phenyl)carbamoyl)-5,6,7,8-tetrahydronaphthalen-2-yl)carbamoyl)-4′-methoxy-[1,1′-biphenyl]-3-carboxylic acid

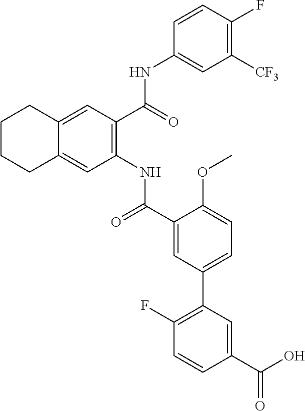 [SMILES: COc1ccc(-c2cc(C(=O)O)ccc2F)cc1C(=O)Nc1cc2c(cc1C(=O)Nc1ccc(F)c(C(F)(F)F)c1)CCCC2]

Example 1 was prepared by adding DIEA (0.342 mL, 1.96 mmol) and HATU (38.9 mg, 0.102 mmol), respectively, to a solution of 3-amino-N-(4-fluoro-3-(trifluoromethyl)phenyl)-5,6,7,8-tetrahydronaphthalene-2-carboxamide (Intermediate 7-6) (30 mg, 0.085 mmol) and 5′-(tert-butoxycarbonyl)-2′-fluoro-4-methoxy-[1,1′-biphenyl]-3-carboxylic acid, (Intermediate 1-1) (35.4 mg, 0.10 mmol) in ACN (3.4 mL). After 12 h, the reaction mixture was extracted with EtOAc (2×25 mL), washed with H₂O, brine, dried over Na₂SO₄, filtered and concentrated under reduced pressure. The residue was treated with 50% TFA/DCM (1 mL). After 3 h, the reaction mixture was concentrated under reduced pressure and purified by reverse phase preparative HPLC (using gradients of mobile phase A: 5:95 acetonitrile: water with 0.1% trifluoroacetic acid; mobile phase B: 95:5 acetonitrile: water with 0.1% trifluoroacetic acid) to give example 1 (3.3 mg, 6%). ¹H NMR (500 MHz, DMSO-d6) δ 11.62-11.58 (m, 1H), 10.81-10.75 (m, 1H), 8.36-8.29 (m, 2H), 8.21-8.18 (m, 1H), 8.05 (br d, J=6.2 Hz, 2H), 8.00-7.95 (m, 1H), 7.82-7.78 (m, 1H), 7.57-7.50 (m, 2H), 7.45 (br t, J=9.4 Hz, 11-1), 7.38-7.33 (m, 1H), 4.05 (s, 3H), 2.82-2.72 (m, 4H), 1.83-1.71 (m, 4H). Analytical LC-MS: RT=2.11 min; MS (ESI) m/z=623.1 (M+H)⁺; HPLC purity 88%: [Method B].

### Example 2

### 2-(6-fluoro-3′-((3-((4-fluoro-3-(trifluoromethyl)phenyl)carbamoyl)-5,6,7,8-tetrahydro-naphthalen-2-yl)carbamoyl)-4′-methoxy-[1,1′-biphenyl]-3-yl)-2-(tetrahydro-2H-pyran-4-carboxamido)acetic acid (homochiral)

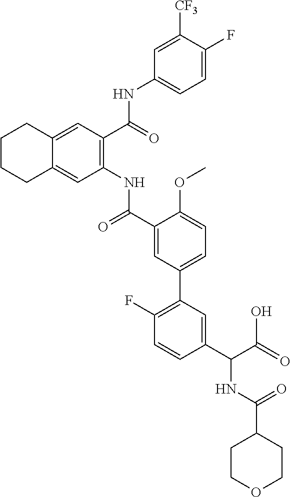 [SMILES: COc1ccc(-c2cc(C(NC(=O)C3CCOCC3)C(=O)O)ccc2F)cc1C(=O)Nc1cc2c(cc1C(=O)Nc1ccc(F)c(C(F)(F)F)c1)CCCC2]

Example 2 was prepared by adding tert-butyl 2-amino-2-(6-fluoro-3′-((3-((4-fluoro-3-(trifluoromethyl)phenyl)carbamoyl)-5,6,7,8-tetrahydronaphthalen-2-yl)carbamoyl)-4′-methoxy-[1,1′-biphenyl]-3-yl)acetate (Intermediate 13-2) (55.4 mg, 0.074 mmol) to DCM (1.5 mL), and treating with DIPEA (0.130 mL, 0.74 mmol) followed by the addition of tetrahydro-2H-pyran-4-carbonyl chloride (11.03 mg, 0.074 mmol). After stirring for 1 h, the solution was concentrated under reduced pressure and purified by reverse phase chromatography (using gradients of mobile phase A: 10% ACN/90% H₂O/0.1% TFA; mobile phase B: 90% ACN/10% H₂O/0.1% TFA) to generate tert-butyl 2-(6-fluoro-3′-((3-((4-fluoro-3-(trifluoromethyl)phenyl) carbamoyl)-5,6,7,8-tetrahydronaphthalen-2-yl)carbamoyl)-4′-methoxy-[1,1′-biphenyl]-3-yl)-2-(tetrahydro-2H-pyran-4-carboxamido)acetate. Analytical LC-MS: RT=1.30 min; MS (ESI) m/z=822.1 (M+H)⁺; [Method A].

The t-butyl group was removed by re-dissolving tert-butyl 2-(6-fluoro-3′-((3-((4-fluoro-3-(trifluoromethyl)phenyl)carbamoyl)-5,6,7,8-tetrahydronaphthalen-2-yl)carbamoyl)-4′-methoxy-[1,1′-biphenyl]-3-yl)-2-(tetrahydro-2H-pyran-4-carboxamido)acetate in DCM (1 mL) and treating with TFA (1 mL). After stirring for 2 h, the reaction mixture was concentrated under reduced pressure and purified by reverse phase chromatography (using gradients of mobile Phase A: 5:95 acetonitrile: water with 0.1% trifluoroacetic acid; mobile Phase B: 95:5 acetonitrile: water with 0.1% trifluoroacetic acid) to give example 2 (6.0 mg, 11% yield). ¹H NMR (500 MHz, DMSO-d6) δ 11.65 (s, 1H), 10.80 (s, 1H), 8.66 (d, J=7.7 Hz, 1H), 8.39-8.32 (m, 2H), 8.21 (s, 1H), 8.09-8.03 (m, 1H), 7.78-7.72 (m, 1H), 7.59-7.52 (m, 3H), 7.46-7.41 (m, 1H), 7.38-7.30 (m, 2H), 7.25-7.03 (m, 1H), 5.44-5.40 (m, 1H), 4.05 (s, 3H), 3.89-3.82 (m, 2H), 3.34-3.25 (m, 1H), 2.82-2.75 (m, 4H), 1.81-1.75 (m, 4H), 1.66-1.53 (m, 4H). Analytical LC-MS: RT=2.04 min; MS (ESI) m/z=766.2 (M+H): HPLC purity 93%: [Method B].

### Example 3

### 2-amino-2-(6-fluoro-3′-((3-((4-fluoro-3-(trifluoromethyl)phenyl)carbamoyl)-5,6,7,8-tetrahydronaphthalen-2-yl)carbamoyl)-4′-methoxy-[1,1′-biphenyl]-3-yl)acetic acid, TFA salt (homochiral)

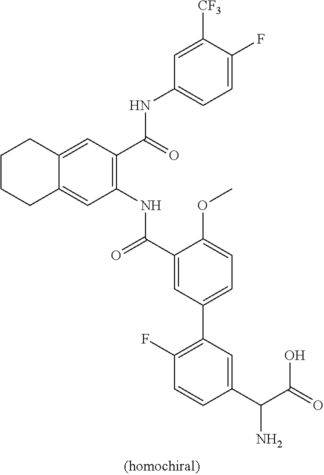 [SMILES: COc1ccc(-c2cc(C(N)C(=O)O)ccc2F)cc1C(=O)Nc1cc2c(cc1C(=O)Nc1ccc(F)c(C(F)(F)F)c1)CCCC2]

Example 3 (1.2 mg, 2% yield) was isolated as the more polar peak during purification of example 2 during reverse phase chromatography (using gradients of mobile Phase A: 5:95 acetonitrile: water with 0.1% trifluoroacetic acid; mobile Phase B: 95:5 acetonitrile: water with 0.1% trifluoroacetic acid). ¹H NMR (500 MHz, DMSO-d6) δ 11.64 (s, 1H), 10.79 (s, 1H), 8.38-8.32 (m, 2H), 8.24 (s, 1H), 8.08-8.04 (m, 1H), 7.78-7.73 (m, 1H), 7.68-7.63 (m, 1H), 7.57-7.52 (m, 2H), 7.46 (br d, J=3.1 Hz, 1H), 7.39-7.32 (m, 2H), 4.74-4.63 (m, 1H), 4.07-4.02 (m, 3H), 2.83-2.75 (m, 4H), 1.82-1.68 (m, 5H). Analytical LC-MS: RT=2.025 min: MS (ESI) m, z=654.1 (M+H)⁺; HPLC purity 99%: [Method B].

### Example 4

### (S)—N-(4-fluoro-3-(trifluoromethyl)phenyl)-3-(2′-fluoro-4-methoxy-5′-(2,2,2-trifluoro-1-hydroxyethyl)-[1,1′-biphenyl]-3-carboxamido)-5,6,7,8-tetrahydronaphthalene-2-carboxamide

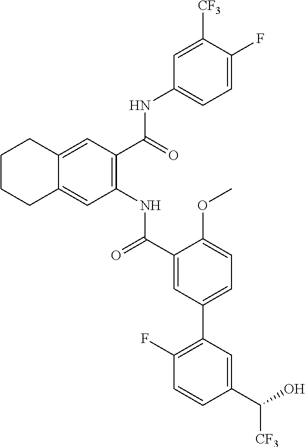 [SMILES: COc1ccc(-c2cc([C@H](O)C(F)(F)F)ccc2F)cc1C(=O)Nc1cc2c(cc1C(=O)Nc1ccc(F)c(C(F)(F)F)c1)CCCC2]

Example 4: Tetrakis(triphenylphosphine)palladium (0) (10.9 mg, 9.43 μmol) was added to a solution of (3-((3-((4-fluoro-3-(trifluoromethyl)phenyl)carbamoyl)-5,6,7,8-tetrahydronaphthalen-2-yl)carbamoyl)-4-methoxyphenyl)boronic acid (Intermediate 15-1) (50 mg, 0.094 mmol), (S)-1-(3-bromo-4-fluorophenyl)-2,2,2-trifluoroethan-1-ol, (Intermediate 14-1) (25.7 mg, 0.0940 mmol), potassium phosphate (60.0 mg, 0.280 mmol), toluene (0.943 mL) sealed and stirred at 80° C. for 14 h. The reaction mixture was allowed to cool to rt, and s extracted with EtOAc (2×25 mL). The organic portion was washed with water, brine, dried over sodium sulfate, filtered, concentrated under reduced pressure and purified by normal phase chromatography using hexanes/EtOAc as eluants to give example 4 (45.3 mg, 71% yield) as a solid. An analytical sample was obtained by further purification by reverse phase chromatography (using gradients of mobile phase A: 5:95 acetonitrile: water with 10 mM ammonium acetate; mobile Phase B: 95:5 acetonitrile: water with 10 mM ammonium acetate). ¹H NMR (500 MHz, DMSO-d6) δ 11.62 (s, 1H), 10.78 (s, 1H), 8.36-8.32 (m, 2H), 8.21-8.19 (m, 1H), 8.08-8.04 (m, 1H), 7.75 (br d, J=8.8 Hz, 1H), 7.66 (br d, J=7.5 Hz, 1H), 7.57-7.51 (m, 3H), 7.40-7.34 (m, 2H), 6.99 (br d, J=5.2 Hz, 1H), 5.31-5.25 (m, 1H), 4.05 (s, 3H), 2.82-2.74 (m, 4H), 1.82-1.74 (m, 4H). Analytical LC-MS: RT=2.75 min; MS (ESI) m, =679.14 (M+H)⁺; HPLC purity 100%; [Method B].

### Example 5

### (S)-2,2,2-trifluoro-1-(6-fluoro-3′-((3-((4-fluoro-3-(trifluoromethyl)phenyl)carbamoyl)-5,6,7,8-tetrahydronaphthalen-2-yl)carbamoyl)-4′-methoxy-[1,1′-biphenyl]-3-yl)ethyl phenylcarbamate

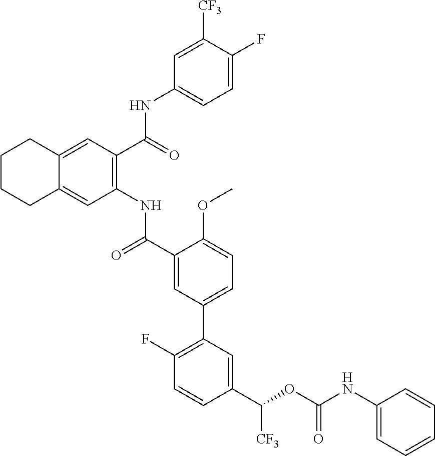 [SMILES: COc1ccc(-c2cc([C@H](OC(=O)Nc3ccccc3)C(F)(F)F)ccc2F)cc1C(=O)Nc1cc2c(cc1C(=O)Nc1ccc(F)c(C(F)(F)F)c1)CCCC2]

Example 5: Phenyl isocyanate (35.1 mg, 0.300 mmol) was added to (S)—N-(4-fluoro-3-(trifluoromethyl)phenyl)-3-(2′-fluoro-4-methoxy-5′-(2,2,2-trifluoro-1-hydroxyethyl)-[1,1′-biphenyl]-3-carboxamido)-5,6,7,8-tetrahydronaphthalene-2-carboxamide (example 4, 20 mg, 0.029 mmol) and pyridine (0.048 mL, 0.59 mmol) in DCM (2.0 mL) and stirred for 14 h. The reaction mixture was quenched with MeOH, concentrated under reduced pressure and purified by reverse phase chromatography (using gradients of mobile phase A: 5:95 acetonitrile: water with 10 mM ammonium acetate: mobile Phase B: 95:5 acetonitrile: water with 10 mM ammonium acetate) to give example 5 (14 mg, 58% yield) as a solid. ¹H NMR (500 MHz, DMSO-d6) δ 11.59-11.57 (m, 1H), 10.73-10.71 (m, 1H), 10.24 (br s, 1H), 8.32-8.27 (m, 2H), 8.19-8.15 (m, 1H), 8.01 (br dd, J=8.1, 3.8 Hz, 1H), 7.78-7.70 (m, 2H), 7.62-7.58 (m, 1H), 7.52-7.46 (m, 2H), 7.45-7.39 (m, 3H), 7.32 (d, J=8.9 Hz, 1H), 7.25 (t, J=7.8 Hz, 2H), 7.02-6.96 (m, 1H), 6.52 (q, J=7.0 Hz, 1H), 4.01 (s, 3H), 2.78-2.69 (m, 4H), 1.78-1.68 (m, 4H). Analytical LC-MS: RT=3.05 min; MS (ESI) m/z=798.3 (M+H)⁺; HPLC purity 99%; [Method B].

### Example 6

### (S)-2,2,2-trifluoro-1-(6-fluoro-3′-((3-((4-fluoro-3-(trifluoromethyl)phenyl)carbamoyl)-5,6,7,8-tetrahydronaphthalen-2-yl)carbamoyl)-4′-methoxy-[1,1′-biphenyl]-3-yl)ethyl cyclobutylcarbamate

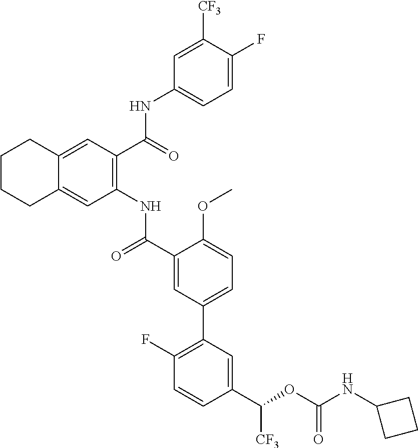 [SMILES: COc1ccc(-c2cc([C@H](OC(=O)NC3CCC3)C(F)(F)F)ccc2F)cc1C(=O)Nc1cc2c(cc1C(=O)Nc1ccc(F)c(C(F)(F)F)c1)CCCC2]

Example 6: A solution of (S)—N-(4-fluoro-3-(trifluoromethyl)phenyl)-3-(2′-fluoro-4-methoxy-5′-(2,2,2-trifluoro-1-hydroxyethyl)-[1,1′-biphenyl]-3-carboxamido)-5,6,7,8-tetrahydronaphthalene-2-carboxamide (example 4, 20 mg, 0.029 mmol) and pyridine (0.024 mL, 0.30 mmol) in DCM (2.0 mL) was treated with 4-nitrophenyl carbonochloridate (29.7 mg, 0.150 mmol) followed by DMAP (3.6 mg, 0.029 mmol) and stirred for 14 h. Cyclobutanamine (0.025 mL, 0.30 mmol) was added to the solution and the resulting reaction mixture allowed to stir 1 h before concentrating under reduced pressure and purifying by reverse phase chromatography (using gradients of mobile phase A: 5:95 acetonitrile: water with 0.05% trifluoroacetic acid; mobile phase B: 95:5 acetonitrile: water with 0.05% trifluoroacetic acid) to give example 6 (14 mg, 60% yield) as a solid. ¹H NMR (500 MHz, DMSO-d6) δ 11.63-11.61 (m, 1H), 10.78-10.75 (m, 1H), 8.37-8.31 (m, 2H), 8.21-8.18 (m, 1H), 8.17-8.13 (m, 1H), 8.08-8.03 (m, 1H), 7.76-7.69 (m, 2H), 7.57-7.51 (m, 3H), 7.46-7.42 (m, 1H), 7.38-7.33 (m, 1H), 6.41-6.34 (m, 1H), 4.06 (s, 3H), 3.94 (dq, J=16.4, 8.4 Hz, 1H), 2.83-2.74 (m, 4H), 2.18-2.07 (m, 2H), 2.01-1.85 (m, 2H), 1.78 (br s, 4H), 1.60-1.53 (m, 2H). Analytical LC-MS: 2.99 min; MS (ESI) m/z=888.15 (M+H); HPLC purity 99%. [Method B].

TABLE 4

| | | | MS | | LC |
|---|---|---|---|---|---|
| | | | (ESI) | | RT |
| Ex. | | | (M + | | Meth. |
| No. | Structure | Name | H)⁺ | ¹H NMR | (min) |
| 7 | 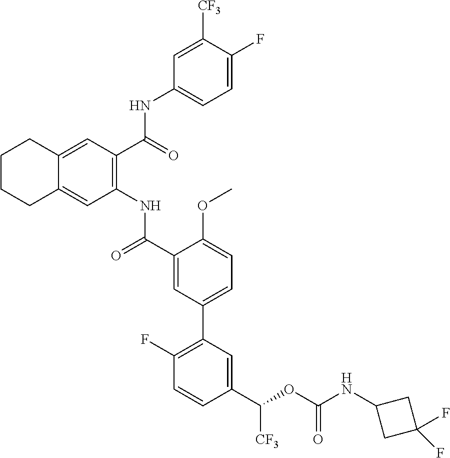 [SMILES: COc1ccc(-c2cc([C@H](OC(=O)NC3CC(F)(F)C3)C(F)(F)F)ccc2F)cc1C(=O)Nc1cc2c(cc1C(=O)Nc1ccc(F)c(C(F)(F)F)c1)CCCC2] | (S)-2,2,2-trifluoro- 1-(6-fluoro-3′-((3- ((4-fluoro-3- (trifluoro- methyl)phenyl) carbamoyl)-5,6,7,8- tetra- hydronaphthalen-2- yl)carbamoyl)-4′- methoxy-[1,1′- biphenyl]-3- yl)ethyl (3,3- difluorocyclo- butyl)carbamate | 812.1 | ¹H NMR (500 MHz, DMSO- d6) δ 11.63- 11.61 (m, 1H), 10.78-10.75 (m, 1H), 8.37- 8.31 (m, 2H), 8.21-8.18 (m, 1H), 8.17- 8.13 (m, 1H), 8.08-8.03 (m, 1H), 7.76- 7.69 (m, 2H), 7.57-7.51 (m, 3H), 7.46- 7.42 (m, 1H), 7.38-7.33 (m, 1H), 6.41- 6.34 (m, 1H), 4.06 (s, 3H), 3.94 (dq, J = 16.4, 8.4 Hz, 1H), 2.83- 2.74 (m, 4H), 2.18-2.07 (m, 2H), 2.01- 1.85 (m, 2H), 1.78 (br s, 4H), 1.60-1.53 (m, 2H) | 2.91, B |
| 8 | 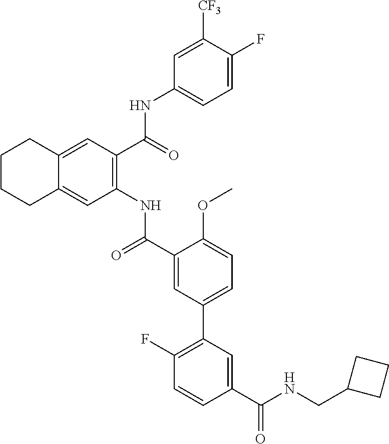 [SMILES: COc1ccc(-c2cc(C(=O)NCC3CCC3)ccc2F)cc1C(=O)Nc1cc2c(cc1C(=O)Nc1ccc(F)c(C(F)(F)F)c1)CCCC2] | N3′-(cyclobutyl- methyl)-6′-fluoro- N3-(3-((4-fluoro-3- (trifluoromethyl) phenyl)carbamoyl)- 5,6,7,8-tetrahydro- naphthalen-2-yl)-4- methoxy-[1,1′- biphenyl]-3,3′- dicarboxamide | 692.1 | ¹H NMR (500 MHz, DMSO- d6) δ 11.65- 11.60 (m, 1H), 10.80-10.76 (m, 1H), 8.58 (br t, J = 5.5 Hz, 1H), 8.38- 8.32 (m, 2H), 8.24 (s, 1H). 8.09-7.99 (m, 2H), 7.91- 7.86 (m, 1H), 7.80 (br d, J = 8.5 Hz, 1H), 7.59-7.52 (m, 2H), 7.43- 7.35 (m, 2H), 4.06 (s, 3H), 2.82-2.73 (m, 4H), 2.57- 2.54 (m, 3H), 1.99 (br dd, J = 9.3, 6.6 Hz, 2H), 1.86- 1.67 (m, 8H). | 2.84, B |
| 9 | 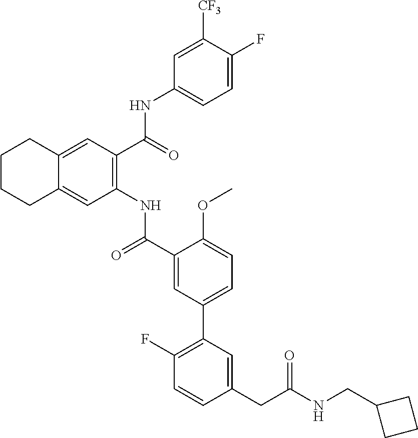 [SMILES: COc1ccc(-c2cc(CC(=O)NCC3CCC3)ccc2F)cc1C(=O)Nc1cc2c(cc1C(=O)Nc1ccc(F)c(C(F)(F)F)c1)CCCC2] | 3-(5′-(2- ((cyclobutylmethyl) amino)-2- oxoethyl)-2′- fluoro-4-methoxy- [1,1′-biphenyl]-3- carboxamido)-N- (4-fluoro-3- (trifluoromethyl) phenyl)-5,6,7,8- tetrahydronaphthalene- 2-carboxamide | 706.4 | ¹H NMR (500 MHz, DMSO- d6) δ 11.64- 11.61 (m, 1H), 10.77 (s, 1H), 8.36-8.32 (m, 2H), 8.19 (s, 1H), 8.08-8.05 (m, 2H), 7.74- 7.71 (m, 1H), 7.56-7.51 (m, 2H), 7.42 (br d, J = 6.1 Hz, 1H), 7.33 (d, J = 8.5 Hz, 1H), 7.28-7.21 (m, 2H), 4.07- 4.02 (m, 3H), 3.10-3.05 (m, 2H), 2.81- 2.74 (m, 4H), 2.41-2.36 (m, 1H), 1.95- 1.89 (m, 2H), 1.82-1.73 (m, 7H), 1.65- 1.58 (m, 2H). | 2.87, B |
| 12 | 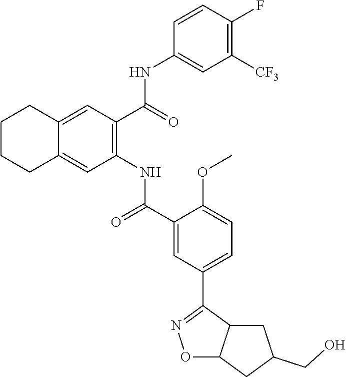 [SMILES: COc1ccc(C2=NOC3CC(CO)CC23)cc1C(=O)Nc1cc2c(cc1C(=O)Nc1ccc(F)c(C(F)(F)F)c1)CCCC2] (diastereomenic mixture) | N-(4-fluoro-3- (trifluoromethyl) phenyl)-3-(5-(5- (hydroxymethyl)- 3a,5,6,6a- tetrahydro-4H- cyclopenta[d]- isoxazol-3-yl)-2- methoxybenzamido)- 5,6,7,8- tetrahydronaphthalene- 2-carboxamide | 624 | ¹H NMR (500 MHz, DMSO- d6) δ 11.54 (br s, 1H), 10.75 (s, 1H), 8.28 (br s, 2H), 8.04- 8.00 (m, 1H), 7.83 (br d, J = 9.4 Hz, 1H), 7.53-7.49 (m, 2H), 7.29 (br d, J = 8.6 Hz, 1H), 5.16- 5.10 (m, 1H), 4.19 (brt, J = 8.3 Hz, 1H), 4.04-3.96 (m, 3H), 3.40- 3.12 (m, 2H), 2.80-2.71 (m, 4H), 2.23- 2.08 (m, 1H), 2.02-1.84 (m, 2H), 1.76 (br s, 5H), 1.69-1.42 | 2.47, B |
| 13 | 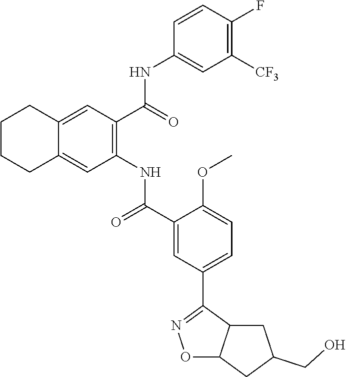 [SMILES: COc1ccc(C2=NOC3CC(CO)CC23)cc1C(=O)Nc1cc2c(cc1C(=O)Nc1ccc(F)c(C(F)(F)F)c1)CCCC2] (homochiral) Prepared from intermediate 6-8 (homochiral, peak 3) | N-(4-fluoro-3- (trifluoromethyl) phenyl)-3-(5-(5- (hydroxymethyl)- 3a,5,6,6a- tetrahydro-4H- cyclopenta[d]- isoxazol-3-yl)-2- methoxybenzamido)- 5,6,7,8- tetrahydronaphthalene- 2-carboxamide | 624 | ¹H NMR (500 MHz, DMSO- d6) δ 11.60- 11.55 (m, 1H), 10.78-10.73 (m, 1H), 8.35- 8.26 (m, 3H), 8.08-8.00 (m, 1H), 7.87- 7.82 (m, 1H), 7.56-7.50 (m, 2H), 7.31- 7.27 (m, 1H), 5.13 (br dd, J = 8.2, 5.2 Hz, 1H), 4.57- 4.50 (m, 1H), 4.24-4.15 (m, 1H), 4.03 (s, 3H), 3.45-3.35 (m, 2H), 2.77 (br d, J = 2.7 Hz, 4H), 2.03- 1.96 (m, 1H), 1.93-1.87 (m, 1H), 1.84- 1.74 (m, 5H), 1.70-1.63 (m, 1H), 1.59- 1.52 (m, 1H). | 2.40, B |
| 14 | 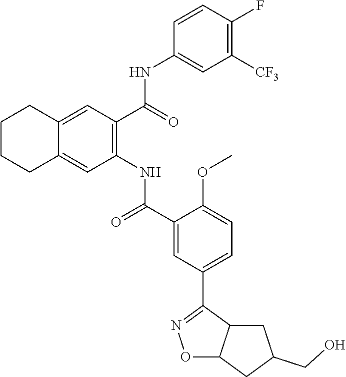 [SMILES: COc1ccc(C2=NOC3CC(CO)CC23)cc1C(=O)Nc1cc2c(cc1C(=O)Nc1ccc(F)c(C(F)(F)F)c1)CCCC2] (homochiral) Prepared from intermediate 6-10 (homochiral, peak 4) | N-(4-fluoro-3- (trifluoromethyl) phenyl)-3-(5-(5- (hydroxymethyl)- 3a,5,6,6a- tetrahydro-4H- cyclopenta[d]- isoxazol-3-yl)-2- methoxybenzamido)- 5,6,7,8- tetrahydronaphthalene- 2-carboxamide | 624 | 1H NMR (500 MHz, DMSO- d6) δ 11.58 (s, 1H), 10.76 (s, 1H), 8.34- 8.27 (m, 3H), 8.07-8.03 (m, 1H), 7.86- 7.82 (m, 1H), 7.56-7.50 (m, 2H), 7.31- 7.28 (m, 1H), 5.18-5.12 (m, 1H), 4.08- 4.08 (m, 1H), 4.11 (td, J = 9.4, 6.6 Hz, 1H), 4.03 (s, 3H), 3.38-3.19 (m, 2H), 2.81- 2.72 (m, 4H), 2.25-2.10 (m, 3H), 1.83- 1.72 (m, 4H), 1.67-1.60 (m, 1H), 1.50- 1.43 (m, 1H) | 2.40, B |
| 15 | 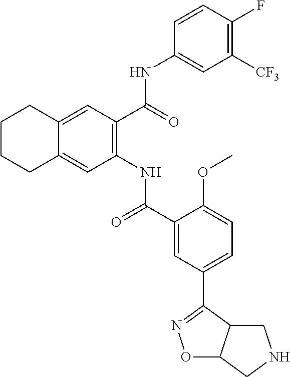 [SMILES: COc1ccc(C2=NOC3CNCC23)cc1C(=O)Nc1cc2c(cc1C(=O)Nc1ccc(F)c(C(F)(F)F)c1)CCCC2] (diastereomeric mixture) | N-(4-fluoro-3- (trifluoromethyl) phenyl)-3-(2- methoxy-5- (3a,5,6,6a- tetrahydro-4H- pyrrolo[3,4- d]isoxazol-3- yl)benzamido)- 5,6,7,8- tetrahydronaphthalene- 2-carboxamide, TFA salt | 597.1 | ¹H NMR (500 MHz, DMSO- d6) δ 11.59 (s, 1H), 10.78 (s, 1H), 8.31 (s, 3H), 8.03 (br dd, J = 6.7, 2.7 Hz, 1H), 7.86 (br d, J = 8.5 Hz, 1H), 7.55- 7.50 (m, 2H), 7.32 (d, J = 8.9 Hz, 1H), 5.46- 5.41 (m, 1H), 4.66 (br t, J = 9.3 Hz, 1H), 4.04 (s, 3H), 3.59-3.25 (m, 2H), 2.79- 2.73 (m, 4H), 1.81-1.72 (m, 4H). | 2.31, B |
| 16 | 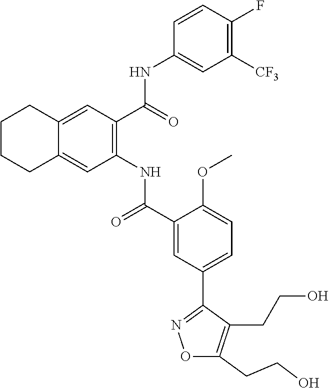 [SMILES: COc1ccc(-c2noc(CCO)c2CCO)cc1C(=O)Nc1cc2c(cc1C(=O)Nc1ccc(F)c(C(F)(F)F)c1)CCCC2] | 3-(5-(4,5-bis(2- hydroxyethyl) isoxazol-3-yl)-2- methoxybenzamido)- N-(4-fluoro-3- (trifluoromethyl) phenyl)-5,6,7,8- tetrahydronaphthalene- 2-carboxamide | 640.2 | ¹H NMR (500 MHz, DMSO- d6) δ 11.61 (s, 1H), 10.76 (s, 1H), 8.46 (d, J = 2.1 Hz, 1H), 8.37-8.30 (m, 2H), 8.10- 8.01 (m, 1H), 8.00 (dd, J = 8.7, 2.3 Hz, 1H), 7.57-7.51 (m, 2H), 7.34 (d, J = 8.9 Hz, 1H), 6.86-6.81 (m, 1H), 4.05 (s, 3H), 3.76 (t, J = 6.4 Hz, 2H), 3.53-3.45 (m, 2H), 3.05 (t, J = 6.3 Hz, 2H), 2.79 (br dd, J = 6.4, 3.4 Hz, 4H), 1.78 (br s, 4H). | 2.48, B |
| 17 | 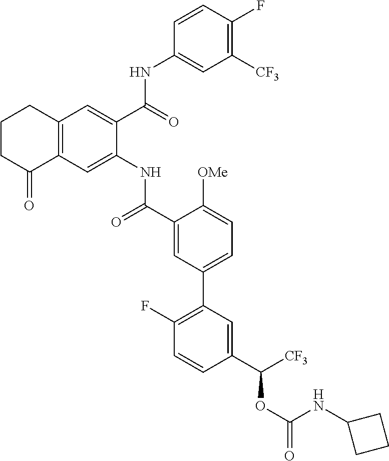 [SMILES: COc1ccc(-c2cc([C@H](OC(=O)NC3CCC3)C(F)(F)F)ccc2F)cc1C(=O)Nc1cc2c(cc1C(=O)Nc1ccc(F)c(C(F)(F)F)c1)CCCC2=O] | (S)-2,2,2-trifluoro- 1-(6-fluoro-3′-((3- (4-fluoro-3- (trifluoromethyl) phenyl)carbamoyl)-8- oxo-5,6,7,8- tetrahydronaphthalen- 2- yl)carbamoyl)-4′- methoxy-[1,1′- biphenyl]-3- yl)ethyl cyclobutylcarbamate | 790.4 | ¹H NMR (500 MHz, DMSO- d6) δ 11.47 (s, 1H), 11.05 (s, 1H), 9.02 (s, 1H), 8.35 (br d, J = 4.3 Hz, 1H), 8.22 (s, 1H), 8.18-8.13 (m, 1H), 8.10- 8.05 (m, 1H), 7.80 (s, 1H), 7.77 (br d, J = 8.2 Hz, 1H), 7.72 (br d, J = 6.7 Hz, 1H), 7.59-7.54 (m, 2H), 7.47- 7.41 (m, 1H), 7.37 (d, J = 8.9 Hz, 1H), 6.38 (q, J = 6.5 Hz, 1H), 4.05 (s, 3H), 3.97-3.90 (m, 1H), 3.01 (br t, J = 5.2 Hz, 2H), 2.69 (br t, J = 5.8 Hz, 2H), 2.17- 2.06 (m, 4H), 1.97-1.85 (m, 2H), 1.63- 1.52 (m, 2H). | 2.74, C |
| 18 | 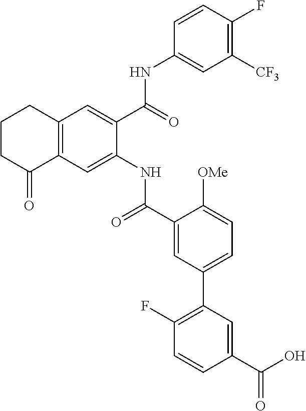 [SMILES: COc1ccc(-c2cc(C(=O)O)ccc2F)cc1C(=O)Nc1cc2c(cc1C(=O)Nc1ccc(F)c(C(F)(F)F)c1)CCCC2=O] | 6-fluoro-3′-((3-((4- fluoro-3- (trifluoromethyl) phenyl)carbamoyl)-8- oxo-5,6,7,8- tetrahydronaphthalen- 2- yl)carbamoyl)-4′- methoxy-[1,1′- biphenyl]-3- carboxylic acid | 639.3 | ¹H NMR (500 MHz, DMSO- d6) δ 13.22- 13.12 (m, 1H), 11.47-11.43 (m, 1H), 11.03 (s, 1H), 9.01 (s, 1H), 8.34 (dd, J = 6.5, 2.4 Hz, 1H), 8.22 (d, J = 0.9 Hz, 1H), 8.09-8.04 (m, 2H), 7.97 (ddd, J = 8.4, 4.9, 2.1 Hz, 1H), 7.83-7.77 (m, 2H), 7.57 (t, J = 9.8 Hz, 1H), 7.44 (br t, J = 9.5 Hz, 1H), 7.36 (d, J = 8.9 Hz, 1H), 4.05 (s, 3H), 3.01 (br t, J = 5.8 Hz. 2H), 2.69 (br t, J = 6.4 Hz, 2H), 2.14- 2.07 (m, 2H). | 1.30, A |
| 19 | 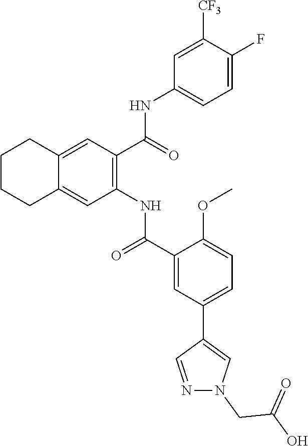 [SMILES: COc1ccc(-c2cnn(CC(=O)O)c2)cc1C(=O)Nc1cc2c(cc1C(=O)Nc1ccc(F)c(C(F)(F)F)c1)CCCC2] | 2-(4-(3-((3-((4- fluoro-3- (trifluoromethyl) phenyl)carbamoyl)- 5,6,7,8- tetrahydronaphthalen- 2- yl)carbamoyl)-4- methoxyphenyl)- 1H-pyrazol-1- yl)acetic acid | 611.1 | ¹H NMR (500 MHz): δ = 1.78 (br s, 4H, M13), 2.74- 2.81 (m, 4H, M12), 3.98 (s, 3H, M11), 4.54 (s, 2H, M10), 7.20 (d, 1H, J = 8.5 Hz, M09), 7.51-7.56 (m, 2H, M08), 7.70-7.73 (m, 2H, M07), 8.01 (s, 1H, M03), 8.04-8.09 (m, 1H, M02), 8.14 (d, 1H, J = 2.1 Hz, M01), 8.34 (dd, 1H, J = 6.6, 2.3 Hz, M04), 8.37 (s, 1H, M05), 11.57 (s, 1H, M06) | 2.05, B |
| 20 | 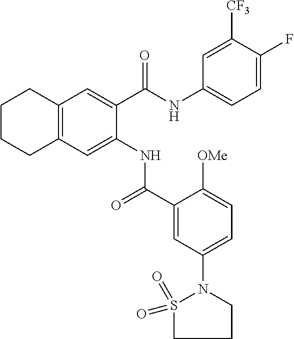 [SMILES: COc1ccc(N2CCCS2(=O)=O)cc1C(=O)Nc1cc2c(cc1C(=O)Nc1ccc(F)c(C(F)(F)F)c1)CCCC2] | 3-(5-(1,1- dioxidoisothiazolidin- 2-yl)-2- methoxybenzamido)- N-(4-fluoro-3- (trifluoromethyl)- phenyl)-5,6,7,8- tetrahydronaphthalene- 2-carboxamide | 606.0 | ¹H NMR (500 MHz, DMSO- d6) δ 11.55 (s, 1H), 10.75 (s, 1H), 8.31- 8.26 (m, 2H), 8.03-7.99 (m, 1H), 7.88 (d, J = 3.1 Hz, 1H), 7.53-7.47 (m, 2H), 7.38 (dd, J = 8.9, 3.1 Hz, 1H), 7.24 (d, J = 9.2 Hz, 1H), 3.96 (s, 3H), 3.67-3.61 (m, 3H), 3.45 (br t, J = 7.5 Hz, 1H), 2.81- 2.72 (m, 4H), 2.43-2.35 (m, 2H), 1.76 (br s, 4H) | 2.43, C |
| 21 | 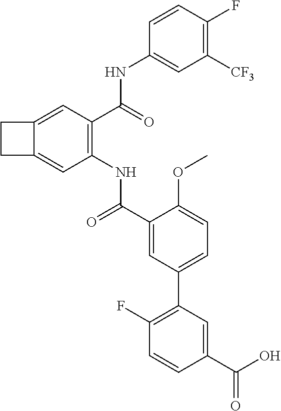 [SMILES: COc1ccc(-c2cc(C(=O)O)ccc2F)cc1C(=O)Nc1cc2c(cc1C(=O)Nc1ccc(F)c(C(F)(F)F)c1)CC2] | 6-fluoro-3′-((4-((4- fluoro-3- (trifluoromethyl) phenyl)carbamoyl) bicyclo[4.2.0]octa- 1,3,5-trien-3- yl)carbamoyl)-4- methoxy-[1,1′- biphenyl]-3- carboxylic acid | 597.1 | ¹H NMR (500 MHz, DMSO- d6) δ 11.54- 11.52 (m, 1H), 10.80 (s, 1H), 8.35-8.33 (m, 1H), 8.21 (br d, J = 17.7 Hz, 2H), 8.07- 8.02 (m, 2H), 7.99-7.94 (m, 2H), 7.79 (br d, J = 8.2 Hz, 1H), 7.55-7.50 (m, 2H), 7.44 (dd, J = 10.2, 8.7 Hz, 1H), 7.35 (d, J = 8.5 Hz, 1H), 4.04 (s, 3H), 2.89 (s, 2H), 2.73 (s, 2H). | 1.99, B |
| 22 | 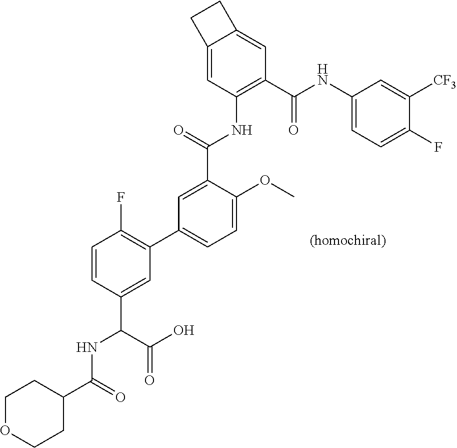 [SMILES: COc1ccc(-c2cc(C(NC(=O)C3CCOCC3)C(=O)O)ccc2F)cc1C(=O)Nc1cc2c(cc1C(=O)Nc1ccc(F)c(C(F)(F)F)c1)CC2] | 2-(6-fluoro-3′-((4- ((4-fluoro-3- (trifluoromethyl) phenyl)carbamoyl) bicyclo[4.2.0]octa- 1,3,5-trien-3- yl)carbamoyl)-4′- methoxy-[1,1′- biphenyl]-3-yl)-2- (tetrahydro-2H- pyran-4- carboxamido)acetic acid | 738.1 | ¹H NMR (500 MHz, DMSO- d6) δ 11.50- 11.47 (m, 1H), 10.80-10.76 (m, 1H), 8.54 (br d, J = 3.7 Hz, 1H), 8.32- 8.27 (m, 1H), 8.16 (s, 1H), 8.01-7.97 (m, 1H), 7.74- 7.69 (m, 1H), 7.53-7.46 (m, 2H), 7.42- 7.37 (m, 1H), 7.35-7.25 (m, 2H), 5.34- 5.29 (m, 1H), 4.00 (s, 2H), 3.87-3.79 (m, 4H), 3.69 (br s, 3H), 3.28 (br dd, J = 11.4, 3.2 Hz, 2H), 3.20 (br d, J = 9.2 Hz, 3H), 1.62- 1.50 (m, 4H) | 1.92, B |
| 23 | 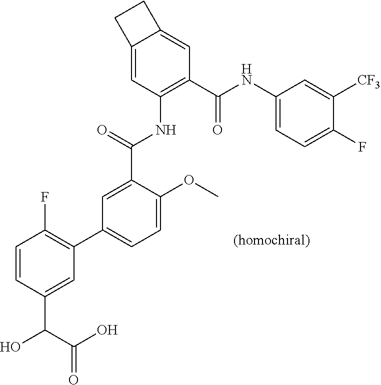 [SMILES: COc1ccc(-c2cc(C(O)C(=O)O)ccc2F)cc1C(=O)Nc1cc2c(cc1C(=O)Nc1ccc(F)c(C(F)(F)F)c1)CC2] | 2-(6-fluoro-3′-((4- ((4-fluoro-3- (trifluoromethyl) phenyl)carbamoyl) bicyclo[4.2.0]octa- 1,3,5-trien-3- yl)carbamoyl)-4′- methoxy-[1,1′- biphenyl]-3-yl)-2- hydroxyacetic acid | 627.1 | ¹H NMR (500 MHz, DMSO- d6) δ 11.52- 11.49 (m, 1H), 10.81-10.79 (m, 1H), 8.35- 8.30 (m, 1H), 8.21 (s, 1H), 8.17-8.14 (m, 1H), 8.06- 8.00 (m, 1H), 7.74-7.69 (m, 1H), 7.56- 7.47 (m, 3H), 7.40-7.37 (m, 1H), 7.32 (br d, J = 8.9 Hz, 1H), 7.20-7.15 (m, 1H), 4.65- 4.60 (m, 1H), 4.04-3.97 (m, 3H), 3.53- 3.47 (m, 2H), 3.27-3.15 (m, 4H). | 1.86, B |
| 24 | 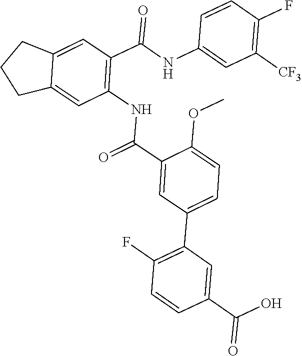 [SMILES: COc1ccc(-c2cc(C(=O)O)ccc2F)cc1C(=O)Nc1cc2c(cc1C(=O)Nc1ccc(F)c(C(F)(F)F)c1)CCC2] | 6-fluoro-3′-((6-((4- fluoro-3- (trifluoromethyl) phenyl)carbamoyl)- 2,3-dihydro-1H- inden-5- yl)carbamoyl)-4′- methoxy-[1,1′- biphenyl]-3- carboxylic acid | 611.0 | ¹H NMR (500 MHz, DMSO- d6) δ 10.67 (s, 1H), 10.62 (s, 1H), 8.23 (br d, J = 4.0 Hz, 1H), 8.03- 7.95 (m, 4H), 7.79-7.74 (m, 1H), 7.53- 7.40 (m, 3H), 7.35 (d, J = 8.9 Hz, 1H), 7.28 (d, J = 7.9 Hz, 1H), 4.05 (s, 2H), 2.98 (br t, J = 7.3 Hz, 2H), 2.86 (br t, J = 7.2 Hz, 2H), 2.09-2.01 (m, 2H). | 1.82, B |
| 25 | 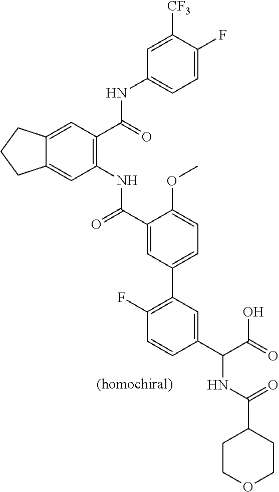 [SMILES: COc1ccc(-c2cc(C(NC(=O)C3CCOCC3)C(=O)O)ccc2F)cc1C(=O)Nc1cc2c(cc1C(=O)Nc1ccc(F)c(C(F)(F)F)c1)CCC2] | 2-(6-fluoro-3′-((6- ((4-fluoro-3- (trifluoromethyl) phenyl)carbamoyl)- 2,3-dihydro-1H- inden-5- yl)carbamoyl)-4′- methoxy-[1,1′- biphenyl]-3-yl)-2- (tetrahydro-2H- pyran-4- carboxamido)acetic acid | 752.2 | ¹H NMR (500 MHz, DMSO- d6) δ 11.65- 11.62 (m, 1H), 10.80-10.78 (m, 1H), 8.49 (br d, J = 7.6 Hz, 1H), 8.46- 8.45 (m, 1H), 8.46-8.45 (m, 1H), 8.36- 8.32 (m, 1H), 8.20-8.16 (m, 1H), 8.05 (br dd, J = 5.2, 3.7 Hz, 1H), 7.73 (br d, J = 8.9 Hz, 1H), 7.68- 7.66 (m, 1H), 7.55-7.50 (m, 2H), 7.42- 7.37 (m, 1H), 7.34 (d, J = 8.5 Hz, 1H), 7.31- 7.25 (m, 1H), 5.31 (d, J = 7.6 Hz, 1H), 4.04 (s, 3H), 3.87- 3.80 (m, 2H), 3.29 (qd, J = 11.2, 3.4 Hz, 1H), 2.97- 2.88 (m, 4H), 2.59-2.54 (m, 1H), 2.11- 2.04 (m, 2H), 1.63-1.50 (m, 4H). | 1.95, B |
| 26 | 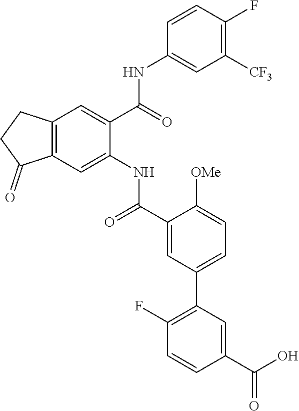 [SMILES: COc1ccc(-c2cc(C(=O)O)ccc2F)cc1C(=O)Nc1cc2c(cc1C(=O)Nc1ccc(F)c(C(F)(F)F)c1)CCC2=O] | 6-fluoro-3′-((6-((4- fluoro-3- (trifluoromethyl) phenyl)carbamoyl)-3- oxo-2,3-dihydro- 1H-inden-5- yl)carbamoyl)-4′- methoxy-[1,1′- biphenyl]-3- carboxylic acid | 625.2 | ¹H NMR (500 MHz, DMSO- d6) δ 11.49 (s, 1H), 11.22- 10.99 (m, 1H), 8.84-8.67 (m, 1H), 8.35 (br d, J = 4.2 Hz, 1H), 8.22 (s, 1H), 8.05 (br d, J = 5.7 Hz, 1H), 8.02-7.96 (m, 1H), 7.81 (br d, J = 8.4 Hz, 1H), 7.56 (br t, J = 9.7 Hz, 1H), 7.45 (br t, J = 9.6 Hz, 1H), 7.36 (d, J = 8.7 Hz, 1H), 4.04 (s, 3H), 3.17 (br d, J = 5.5 Hz, 2H), 3.00 (s, 1H), 2.76 (br d, J = 5.6 Hz, 1H), 2.60- 2.53 (m, 4H). | 1.79, B |
| 27 | 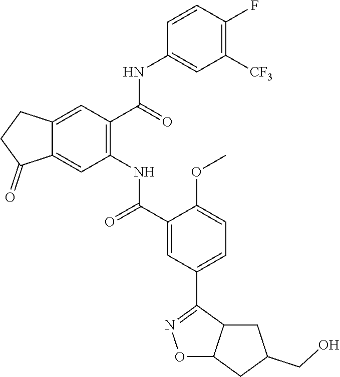 [SMILES: COc1ccc(C2=NOC3CC(CO)CC23)cc1C(=O)Nc1cc2c(cc1C(=O)Nc1ccc(F)c(C(F)(F)F)c1)CCC2=O] (homochiral) Prepared from intermediate 6-8 (homochiral, peak 3) | N-(4-fluoro-3- (trifluoromethyl) phenyl)-6-(5-(5- (hydroxy methyl)- 3a,5,6,6a- tetrahydro-4H- cyclopenta[d] isoxazol-3-yl)-2- methoxybenzamido)- 1-oxo-2,3- dihydro-1H- indene-5- carboxamide (homochiral, peak 3) | 626.5 | 1H NMR (400 MHz, METHANOL-d4) δ 8.86 (s, 1H), 8.45 (d, J = 2.4 Hz, 1H), 8.32 (dd, J = 6.3, 2.5 Hz, 1H), 8.04-7.93 (m, 4H), 7.41 (t, J = 9.7 Hz, 1H), 7.29 (d, J = 8.8 Hz, 1H), 5.24 (dd, J = 8.6, 5.1 Hz, 1H), 4.31- 4.24 (m, 2H), 3.62-3.51 (m, 3H), 3.29- 3.25 (m, 2H), 2.85-2.80 (m, 2H), 2.23- 2.12 (m, 2H), 2.04-1.98 (m, 1H), 1.85- 1.78 (m, 1H), 1.71-1.65 (m, 1H). | 1.24, A |
| 28 | 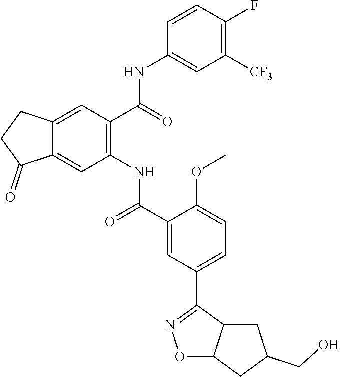 [SMILES: COc1ccc(C2=NOC3CC(CO)CC23)cc1C(=O)Nc1cc2c(cc1C(=O)Nc1ccc(F)c(C(F)(F)F)c1)CCC2=O] (homochiral) Prepared from intermediate 6-10 (homochiral, peak 4) | N-(4-fluoro-3- (trifluoromethyl) phenyl)-6-(5-(5- (hydroxymethyl)- 3a,5,6,6a- tetrahydro-4H- cyclopenta[d] isoxazol-3-yl)-2- methoxybenzamido)- 1-oxo-2,3- dihydro-1H- indene-5- carboxamide (homochiral, peak 4) | | ¹H NMR (400 MHz, METHANOL-d4) δ 8.88- 8.85 (m, 1H), 8.46 (d, J = 2.4 Hz, 1H), 8.34- 8.31 (m, 1H), 8.03-7.94 (m, 3H), 7.41 (t, J = 9.7 Hz, 1H), 7.32-7.28 (m, 1H), 5.30- 5.25 (m, 1H), 4.22-4.17 (m, 1H), 4.14- 4.12 (m, 3H), 3.56-3.44 (m, 3H), 3.31- 3.25 (m, 2H), 2.87-2.80 (m, 2H), 2.44- 2.26 (m, 4H) | 1.27, A |

### Example 29

### 6-fluoro-3′-((6-((4-fluoro-3-(trifluoromethyl)phenyl)carbamoyl)-3-hydroxy-2,3-dihydro-1H-inden-5-yl)carbamoyl)-4′-methoxy-[1,1′-biphenyl]-3-carboxylic acid (racemate)

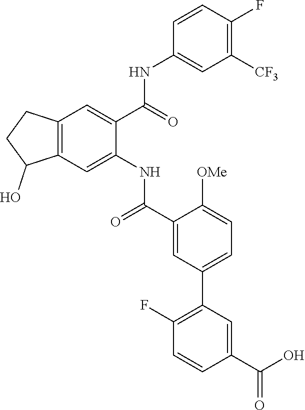 [SMILES: COc1ccc(-c2cc(C(=O)O)ccc2F)cc1C(=O)Nc1cc2c(cc1C(=O)Nc1ccc(F)c(C(F)(F)F)c1)CCC2O]

Example 29: 6-fluoro-3′-((6-((4-fluoro-3-(trifluoromethyl)phenyl)carbamoyl)-3-oxo-2,3-dihydro-1H-inden-5-yl)carbamoyl)-4′-methoxy-[1,1′-biphenyl]-3-carboxylic acid (example 26, 9 mg, 0.013 mmol) was dissolved in THF/MeOH (1:1, 2 mL) and treated with NaBH₄ (2 mg) at rt After 1 h, the reaction mixture was concentrated under reduced pressure, quenched with 1 N HCl, extracted with EtOAc. The combined organic portions were concentrated under reduced pressure. The residue was treated with 50% TFA/DCM (0.25 mL). After 2 h, the reaction mixture was concentrated under reduced pressure and purified by reverse phase chromatography (using gradients of mobile Phase A: 5:95 acetonitrile: water with 0.1% trifluoroacetic acid; mobile Phase B: 95:5 acetonitrile: water with 0.1% trifluoroacetic acid) to give 6-fluoro-3′-((6-((4-fluoro-3-(trifluoromethyl)phenyl)-carbamoyl)-3-hydroxy-2,3-dihydro-1H-inden-5-yl)carbamoyl)-4′-methoxy-[1,1′-biphenyl]-3-carboxylic acid example 29 (1 mg, 11% yield). ¹H NMR (500 MHz, DMSO-d6) δ 11.49 (s, 1H), 11.22-10.99 (m, 1H), 8.84-8.67 (m, 1H), 8.35 (br d, J=4.2 Hz, 1H), 8.22 (s, 1H), 8.05 (br d, J=5.7 Hz, 1H), 8.02-7.96 (m, 1H), 7.81 (br d, J=8.4 Hz, 1H), 7.56 (br t, J=9.7 Hz, 1H), 7.45 (br t, J=9.6 Hz, 1H), 7.36 (d, J=8.7 Hz, 1H), 4.04 (s, 3H), 3.17 (br d, J=5.5 Hz, 2H), 3.00 (s, 1H), 2.76 (br d, J=5.6 Hz, 1H), 2.60-2.53 (m, 4H). Analytical LC-MS: RT=1.76 min; MS (ESI) m/z=627.18 (M+H)⁺; HPLC purity 98%. [Method B].

It will be evident to one skilled in the art that the present disclosure is not limited to the foregoing illustrative examples, and that it can be embodied in other specific forms without departing from the essential attributes thereof. It is therefore desired that the examples be considered in all respects as illustrative and not restrictive, reference being made to the appended claims, rather than to the foregoing examples, and all changes which come within the meaning and range of equivalency of the claims are therefore intended to be embraced therein.

We claim:

1. A compound of Formula (I): 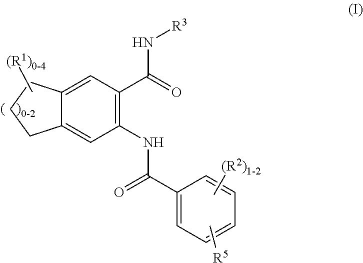
or a pharmaceutically acceptable salt thereof, wherein:
R¹ is halo, C₁₋₄ alkyl substituted with 0-5 halo, ═O, OH, or —OC₁₋₄ alkyl substituted with 0-5 halo;
R² is halo, CN, C₁₋₄ alkyl substituted with 0-5 halo or OH, or —OC₁₋₄ alkyl substituted with 0-5 halo, OH, or —OC₁₋₄ alkyl;
R³ is C₁₋₄ alkyl substituted with 0-5 R⁴, —(CR^{d}R^{d})ₙ, —C₃₋₁₀-carbocyclyl substituted with 0-5 R⁴ or —(CR^{d}R^{d})ₙ-3 to 6-membered heterocyclyl comprising 1-4 heteroatoms selected from O, S(═O)ₚ, N, and NR^{d}, and substituted with 0-5 R⁴;
R⁴ is halo, CN, C₁₋₄ alkyl substituted with 0-5 halo, OH, —OC₁₋₄ alkyl substituted with 0-5 halo, —S(O)ₚR^{c}, aryl, or a 4- to 6-membered heterocyclyl comprising 1-4 heteroatoms selected from O, S(═O)ₚ, N, and NR^{d};
R⁵ is C₃₋₆ carbocyclyl substituted with 0-3 R⁵ and 0-2 R⁷, or a 3- to 12-membered heterocyclyl comprising 1-4 heteroatoms selected from O, S(═O)ₚ, N and NR¹⁰ substituted with 0-3 R⁵ and 0-1 R⁷; wherein said heterocyclyl is bonded to the phenyl moiety through a carbon or nitrogen atom;
R⁶ is halo, CN, ═O, —OH, —OC₁₋₄ alkyl, or C₁₋₄ alkyl substituted with 0-2 halo or OH;
R⁷ is C₁₋₄ alkyl substituted with 0-1 R⁸ and 0-1 R⁹, —OR, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, C₃₋₆ cycloalkyl, or a 4- to 6-membered heterocyclyl comprising 1-4 heteroatoms selected from O, S(═O)ₚ, N and NR^{d}, and substituted with 0-5 R^{c};
R⁸ is halo, —C(═O)OR^{b}, —C(═O)NR^{a}R^{a}, —C(═O)NR^{a}OR^{b}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —NR^{a}S(O)ₚNR^{a}R^{a}, —OC(═O)NR^{a}R^{a}, —OC(═O)NR^{a}OR^{b}, —S(═O)ₚNR^{a}R^{a}, —S(O)ₚR^{c}, —(CH₂)ₙ—C₃₋₆ carbocyclyl substituted with 0-3 R^{e}, or —(CH₂)ₙ-heterocyclyl comprising 1-4 heteroatoms selected from O, S(═O)ₚ, and N, and substituted with 0-3 R^{e};
R¹⁰ is H, C₁₋₄ alkyl substituted with 0-2 R¹¹, —C(═O)R^{b}, —C(═O)OR^{b}, —C(═O)NR^{a}R^{a}, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e};
R¹¹ is —OH, —C(═O)OH, or aryl;
R¹² is H, C₁₋₃ alkyl, or aryl;
R^{a} is H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀ carbocyclyl substituted with 0-5 R^{e}, or —(CH₂)ᵤ-heterocyclyl substituted with 0-5 R^{e}; or R^{a} and R^{a} together with the nitrogen atom to which they are both attached form a heterocyclyl substituted with 0-5 R^{e};
R^{b} is H, C₁₋₆ alkyl substituted with 0-5 R^{e}, C₂₋₆ alkenyl substituted with 0-5 R^{e}, C₂₋₆ alkynyl substituted with 0-5 R^{e}, —(CH₂)ₙ—C₃₋₁₀ carbocyclyl substituted with 0-5 R^{e}, or —(CH₂)ₙ-heterocyclyl substituted with 0-5 R^{e};
R^{c} is C₁₋₆ alkyl substituted with 0-5 R^{e}, C₂₋₄ alkenyl substituted with 0-5 R^{e}, C₂₋₆ alkynyl substituted with 0-5 R^{e}, C₁₋₆ carbocyclyl, or heterocyclyl;
R^{d} is H or C₁₋₄ alkyl;
R^{e} is halo, CN, NO₂, ═O, C₁₋₆ alkyl substituted with 0-5 R^{g}, C₂₋₆ alkenyl substituted with 0-5 R^{g}, C₂₋₆ alkynyl substituted with 0-5 R^{g}, —(CH₂)ⁿ—C₃₋₆ cycloalkyl, —(CH₂)ₙ-aryl, —(CH₂)ₙ-heterocyclyl, —(CH₂)ₙOR^{f}, —C(═O)OR^{f}, —C(═O)NR^{f}R^{f}, —NR^{f}C(═O)R^{f}, —S(═O)ₚR^{f}, —S(═O)ₚNR^{f}R^{f}, —NR^{f}S(═O)ₚR^{f}, —NR^{f}C(═O)OR^{f}, —OC(═O)NR^{f}R^{f}, or —(CH₂)ₙNR^{f}R^{f};
R^{f} is H, C₁₋₆ alkyl, C₃₋₆ cycloalkyl, aryl, or heterocyclyl; or R^{f} and R^{f} together with the nitrogen atom to which they are both attached form a heterocyclyl;
R^{g} is halo, CN, OH, C₁₋₆ alkyl, C₃₋₆ cycloalkyl, or aryl;
n is zero, 1, 2, or 3; and
p is zero, 1, or 2.

2. The compound of claim 1, having Formula (II): 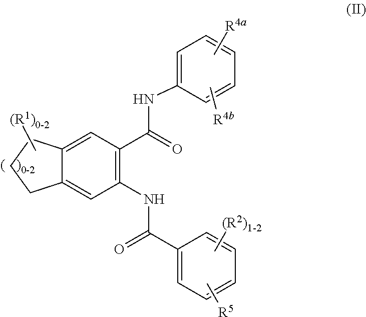
or a pharmaceutically acceptable salt thereof, wherein:
R¹ is halo, ═O, OH, —OC₁₋₄ alkyl substituted with 0-5 halo;
R² is halo, C₁₋₃ alkyl, or —OC₁₋₃ alkyl substituted with 0-4 halo:
R^{4a} is halo;
R^{4b} is C₁₋₄ alkyl substituted with 0-4 halo;
R⁵ is C₆ aryl substituted with 0-3 R⁶ and 0-2 R⁷, or a 3- to 12-membered heterocyclyl comprising 1-4 heteroatoms selected from O, S(═O)ₚ, N and NR¹⁰ substituted with 0-3 R⁶ and 0-1 R⁷;
R⁶ is halo, ═O, —OH, —OC₁₋₄ alkyl, or C₁₋₄ alkyl substituted with 0-2 halo or OH;
R⁷ is C₁₋₃ alkyl substituted with 0-1 R⁸ and 0-1 R⁹, —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, C₃₋₆ cycloalkyl, or a 4- to 6-membered heterocyclyl comprising 1-4 heteroatoms selected from O, S(═O)ₚ, N and NR^{d}, and substituted with 0-5 R^{e};
R⁸ is halo, —C(═O)OR^{b}, —C(═O)NHR^{a}, —C(═O)NHOR^{b}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}S(═O)ₚR^{c}, —NR^{a}S(O)ₚNR^{a}R^{a}, —OC(═O)NR^{a}R^{a}, —OC(═O)NR^{a}OR^{b}, —S(═O)ₚNR^{a}R^{a}, or —S(O)ₚR^{c};
R¹⁰ is H, C₁₋₄ alkyl substituted with 0-2 R^{b}, —C(═O)R^{b}, —C(═O)OR^{b}, —C(═O)NR^{a}R^{a}, C₃₋₄ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e};
R¹¹ is —OH, —C(═O)OH, or aryl;
R¹² is H, C₁₋₃ alkyl, or aryl;
R^{a} is H, C₁₋₅ alkyl substituted with 0-5 R^{e}, C₂₋₅ alkenyl substituted with 0-5 R^{e}, C₂₋₅ alkynyl substituted with 0-5 R^{e}, —(CH₂)ₙ—C₃₋₁₀ carbocyclyl substituted with 0-5 R^{e}, or —(CH₂)ₙ-heterocyclyl substituted with 0-5 R^{e}; or R^{a} and R^{a} together with the nitrogen atom to which they are both attached form a heterocyclyl substituted with 0-5 R^{e};
R^{b} is H, C₁₋₅ alkyl substituted with 0-5 R^{e}, C₂₋₅ alkenyl substituted with 0-5 R^{e}, C₂₋₅ alkynyl substituted with 0-5 R^{e}, —(CH₂)ₙ—C₃₋₁₀carbocyclyl substituted with 0-5 R^{e}, or —(CH₂)ₙ-heterocyclyl substituted with 0-5 R^{e};
R^{c} is C₁₋₅ alkyl substituted with 0-5 R^{e}, C₂₋₅ alkenyl substituted with 0-5 R^{e}, C₂₋₃ alkynyl substituted with 0-5 R^{e}, C₃₋₆ carbocyclyl, or heterocyclyl;
R^{d} is H or C₁₋₄ alkyl;
R^{c} is halo, CN, ═O, C₁₋₆ alkyl substituted with 0-5 R⁹, C₂₋₅ alkenyl substituted with 0-5 R^{g}, C₂₋₆ alkynyl substituted with 0-5 R^{g}, —(CH₂)ₙ—C₃₋₆ cycloalkyl, —(CH₂)ₙ-aryl, —(CH₂)ₙ-heterocyclyl, —(CH₂)ₙOR, or —C(═O)OR^{f};
R^{f} is H or C₁₋₃ alkyl,
R^{g} is halo, CN, OH, C₁₋₆ alkyl, C₃₋₆ cycloalkyl, or aryl;
n is zero, 1, 2, or 3; and
p is zero, 1, or 2.

3. The compound of claim 2, having Formula (III): 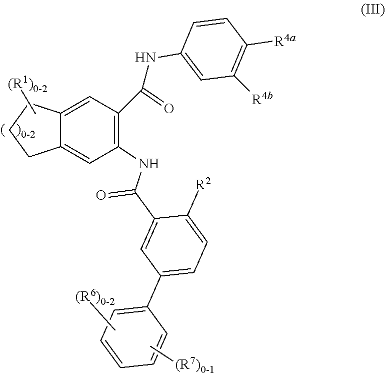
or a pharmaceutically acceptable salt thereof, wherein:
R¹ is OH or ═O;
R² is —OC₁₋₄ alkyl substituted with 0-4 halo;
R^{4a} is halo;
R^{4b} is C₁₋₃ alkyl substituted with 0-4 F;
R⁶ is halo, CN, C₁₋₃ alkyl, —OH, or —OC₁₋₄ alkyl;
R⁷ is C₁₋₂ alkyl substituted with 0-1 R⁸ and 0-1 R⁹, OR^{b}, —NR^{a}R^{a}, —NR^{a}C(═O)R^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, C₃₋₆ cycloalkyl, or a 4- to 6-membered heterocyclyl comprising 1-4 heteroatoms selected from O, S(═O)ₚ, N and NR^{d}, and substituted with 0-4 R^{e};
R^{g} is halo, —C(═O)OR^{b}, —C(═O)NHR^{a}, —C(═O)NHOR^{b}, or C₁₋₄ alkyl substituted with 0-3 halo or OH:
R⁹ is —OR^{b}, —NR^{a}R^{a}, —NR^{a}C(═O)R^{b}, —NR^{a}C(═O)OR^{b}, —NR^{a}S(═O)ₚR^{c}, —NR^{a}S(O)ₚNR^{a}R^{a}, —OC(═O)NR^{a}R^{a}, —OC(═O)NR^{a}OR^{b}, —S(═O)ₚNR^{a}R^{a}, or —S(O)ₚR^{c};
R^{a} is H, C₁₋₅ alkyl substituted with 0-4 R^{e}, C₂₋₅ alkenyl substituted with 0-4 R^{e}, C₂₋₅ alkynyl substituted with 0-4 R^{e}, —(CH₂)ₙ—C₃₋₁₀ carbocyclyl substituted with 0-4 R^{e}, or —(CH₂)ₙ-heterocyclyl substituted with 0-4 R^{e}; or R^{a} and R^{a} together with the nitrogen atom to which they are both attached form a heterocyclyl substituted with 0-4 R^{e}:
R^{b} is H, C₁₋₅ alkyl substituted with 0-4 R^{e}, C₂₋₅ alkenyl substituted with 0-4 R^{e}, C₂₋₅ alkynyl substituted with 0-4 R^{e}, —(CH₂)ₙ—C₃₋₁₀ carbocyclyl substituted with 0-4 R^{e}, or —(CH₂)ₙ-heterocyclyl substituted with 0-4 R^{e};
R^{c} is C₁₋₅ alkyl substituted with 0-4 R^{e}, C₂₋₅ alkenyl substituted with 0-4 R^{e}, C₂₋₅ alkynyl substituted with 0-4 R^{e}, C₃₋₆ carbocyclyl, or heterocyclyl;
R^{d} is H or C₁₋₂ alkyl:
R^{e} is halo, CN, ═O, C₁₋₆ alkyl substituted with 0-5 R⁹, C₂₋₄ alkenyl substituted with 0-5 R^{g}, C₂₋₆ alkynyl substituted with 0-5 R^{g}, —(CH₂)ₙ—C₃₋₆ cycloalkyl, —(CH₂)ₙ-aryl, —(CH₂)ₙ-heterocyclyl, —(CH₂)ₙOR, or —C(═O)OR^{f};
R^{f} is H or C₁₋₃ alkyl,
R^{g} is halo, CN, OH, C₁₋₆ alkyl, or C₃₋₆ cycloalkyl;
n is zero, 1, 2, or 3; and
p is zero, 1, or 2.

4. The compound of claim 3, having Formula (IV): 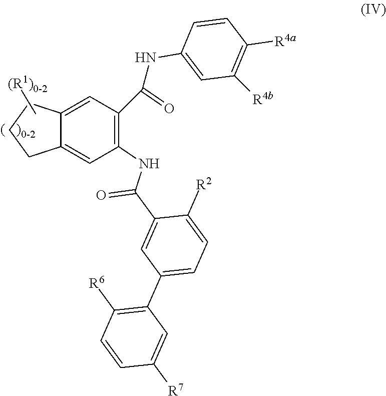
or a pharmaceutically acceptable salt thereof, wherein:
R¹ is OH or ═O;
R² is —OC₁₋₃ alkyl;
R^{4a} is F;
R^{4b} is CF₃;
R⁶ is F;
R⁷ is C₁₋₂ alkyl substituted with 0-1 Rand 0-1 R⁹, —C(═O)OR^{b}, or —C(═O)NR^{a}R^{a};
R⁸ is —C(═O)OR^{b}, —C(═O)NHR^{a}, or C₁₋₄ alkyl substituted with 0-3 halo or OH:
R⁹ is —OR^{b}, —NR^{a}R^{a}, —NR^{a}C(═O)R^{b}, or —OC(═O)NR^{a}R^{a};
R^{a} is H, C₁₋₄ alkyl substituted with 0-3 R^{e}, —(CH₂)ₙ—C₃₋₆ cycloalkyl substituted with 0-3 R^{e}, or phenyl substituted with 0-3 R^{e};
R^{b} is H or heterocyclyl substituted with 0-5 R^{e};
R^{c} is halo, CN, ═O, or C₁₋₆ alkyl; and
n is zero or 1.

5. The compound of claim 4, having Formula (V): 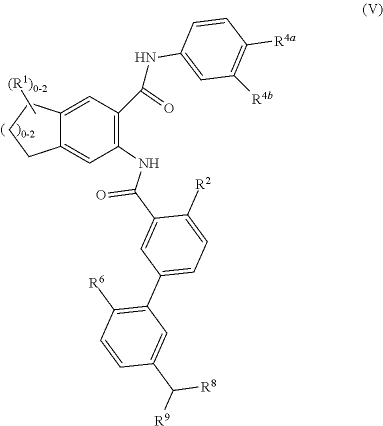
or a pharmaceutically acceptable salt thereof, wherein:
R⁸ is —C(═O)OH or CF₃;
R⁹ is —NHC(═O)R^{b} or —OC(═O)NHR^{a};
R^{a} is —C₃₋₆ cycloalkyl or phenyl; and
R^{b} is heterocyclyl.

6. The compound of claim 2, having Formula (VI): 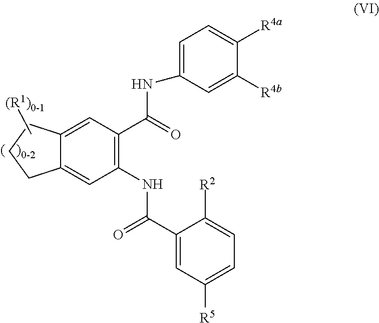
or a pharmaceutically acceptable salt thereof, wherein:
R¹ is ═O;
R² is —OC₁₋₄ alkyl substituted with 0-4 halo:
R^{4a} is halo;
R^{4b} is C₁₋₃ alkyl substituted with 0-4 halo;
R⁵ is a 3- to 12-membered heterocyclyl comprising 1-4 heteroatoms selected from O, S(═O)ₚ, N and NR¹⁰ substituted with 0-3 R⁶ and 0-1 R¹;
R⁶ is halo, ═O, —OH, —OC₁₋₄ alkyl, or C₁₋₄ alkyl substituted with 0-2 halo or OH;
R⁷ is C₁₋₂ alkyl substituted with 0-1 R⁸ and 0-1 R⁹, —OR^{b}, —NR^{a}R^{a}, —NR^{a}C(═O)R^{b}, —NR^{a}C(═O)OR^{b}, —NR^{a}C(═O)NR^{a}R^{a}, —NR³S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR³R^{a}, C₃₋₆ cycloalkyl, or a 4- to 6-membered heterocyclyl comprising 1-4 heteroatoms selected from O, S(═O)ₚ, N and NR^{d}, and substituted with 0-4 R^{e};
R⁸ is —C(═O)OR^{b}, —C(═O)NHR^{a}, —C(═O)NHOR^{b}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}S(═O)ₚR^{c}, —NR^{a}S(O)ₚNR^{a}R^{a}, —OC(═O)NR^{a}R^{a}, —S(═O)ₚNR^{a}R^{a}, or —S(O)ₚR^{c};
R¹⁰ is H, C₁₋₄ alkyl substituted with 0-2 R¹¹, —C(═O)R^{b}, —C(═O)OR^{b}, —C(═O)NR^{a}R^{a}, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e},
R¹¹ is —OH, —C(═O)OH, or aryl;
R¹² is H, C₁₋₃ alkyl, or aryl;
R^{a} is H, C₁₋₅ alkyl substituted with 0-4 R, C₂₋₅ alkenyl substituted with 0-4 R^{e}, C₂₋₅ alkynyl substituted with 0-4 R^{e}, —(CH₂)ₙ—C₃₋₁₀ carbocyclyl substituted with 0-4 R^{e}, or —(CH₂)ₙ-heterocyclyl substituted with 0-4 R^{e}; or R^{a} and R^{a} together with the nitrogen atom to which they are both attached form a heterocyclyl substituted with 0-4 R^{e};
R^{b} is H, C₁₋₅ alkyl substituted with 0-4 R^{e}, C₂₋₅ alkenyl substituted with 0-4 R^{e}, C₂₋₅ alkynyl substituted with 0-4 R^{e}, —(CH₂)ₙ-C₃₋₁₀ carbocyclyl substituted with 0-4 R^{e}, or —(CH₂)ₙ-heterocyclyl substituted with 0-4 R^{e};
R^{c} is C₁₋₅ alkyl substituted with 0-4 R^{e}, C₂₋₅ alkenyl substituted with 0-4 R^{e}, C₂₋₅ alkynyl substituted with 0-4 R^{e}, C₃₋₆ carbocyclyl, or heterocyclyl;
R^{d} is H or C₁₋₂ alkyl:
R^{e} is halo, CN, ═O, C₁₋₆ alkyl substituted with 0-5 R^{g}, C₂₋₆ alkenyl substituted with 0-5 R^{g}, C₂₋₆ alkynyl substituted with 0-5 R^{g}, —(CH₂)ₙ-C₃₋₆ cycloalkyl, —(CH₂)ₙ-aryl, —(CH₂)ₙ-heterocyclyl, —(CH₂)ₙ-heteroaryl, —(CH₂)ₙOR^{f}, or —C(═O)OR^{f};
R^{f} is H or C₁₋₃ alkyl:
R^{g} is halo, CN, OH, C₁₋₆alkyl, or C₃₋₆ cycloalkyl;
n is zero, 1, 2, or 3; and
p is zero, 1, or 2.

7. The compound of claim 6, or a pharmaceutically acceptable salt, thereof, wherein:
R² is —OCH₃:
R^{4a} is F;
R^{4b} is CF₃;
R⁵ is 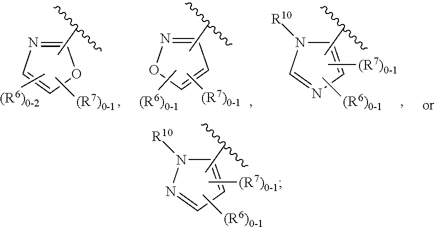
R⁶ is halo, —OH, or C₁₋₄ alkyl substituted with 0-1 OH;
R⁷ is C₁₋₂ alkyl substituted with 0-1 Rand 0-1 R⁹;
R⁸ is —C(═O)OR^{b}, —C(═O)NHR^{a}, or —C(═O)NHOR^{b};
R⁹ is —OR^{b} or —NR^{a}R^{a};
R¹⁰ is H, —C(═O)R^{b}, or C₁₋₄ alkyl substituted with 0-1 R¹¹;
R¹⁰ is —OH, —C(═O)OH, or aryl;
R^{a} is H or C₁₋₃ alkyl; and
R^{b} is H or C₁₋₃ alkyl.

8. The compound of claim 6, or a pharmaceutically acceptable salt thereof, wherein:
R² is —OCH₃;
R^{4a} is F;
R^{4b} is CF₃;
R⁵ is 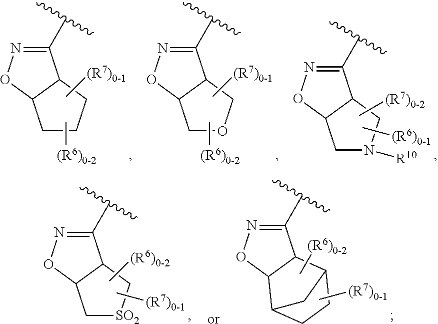
R is halo, C₁₋₄ alkyl, —OH, or —OC₁₋₄ alkyl;
R⁷ is C₁₋₄ alkyl substituted with 0-1 Rand 0-1 R⁹;
R⁸ is —C(═O)OR^{b};
R⁹ is OH;
R¹⁰ is H, C₁₋₃ alkyl substituted with 0-2 R¹¹, or —C(═O)OC₁₋₄ alkyl;
R¹¹ is —OH, —C(═O)OH, or aryl; and
R^{b} is H or C₁₋₄ alkyl.

9. The compound of claim 6, or a pharmaceutically acceptable salt thereof, wherein:
R² is —OCH₃;
R^{4a} is F;
R^{4b} is CF₃;
R⁵ is 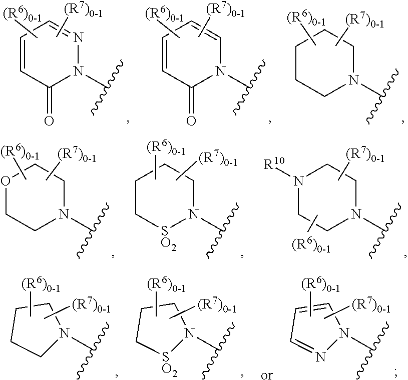
R⁶ is halo, CN, C₁₋₄ alkyl, ═O, —OH, or —OC₁₋₄ alkyl;
R⁷ is C₁₋₂ alkyl substituted with 0-1 Rand 0-1 R⁹, —NR^{a}R^{a}, —NR^{a}C(═O)R^{b}, —NR^{a}C(═O)OR^{b}, or —C(═O)OR^{b};
R⁸ is —C(═O)OR, —C(═O)NHR^{a}, —C(═O)NHOR^{b}, or C₁₋₄ alkyl substituted with 0-3 halo or OH;
R⁹ is —NR^{a}C(═O)R^{b};
R¹⁰ is H or C₁₋₃ alkyl;
R^{a} is H or C₁₋₄ alkyl; and
R^{b} is H, or C₁₋₄ alkyl.

10. The compound of claim 1, having Formula (VII): 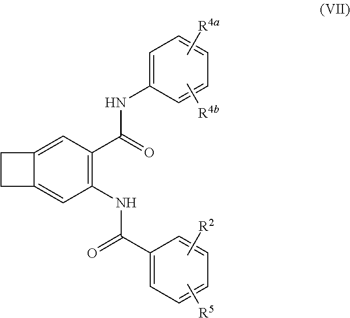
or a pharmaceutically acceptable salt thereof, wherein:
R² is halo, CN, —C(═O)OR^{b}, —NR^{a}R⁸, C₁₋₄ alkyl substituted with 0-5 halo or OH, or —OC₁₋₄ alkyl substituted with 0-4 halo, OH, or —OC₁₋₄ alkyl;
R^{4a} is halo;
R^{4b} is C₁₋₄ alkyl substituted with 0-4 halo;
R⁵ is C₆ aryl substituted with 0-3 R⁶ and 0-2 R⁷, or a 3- to 12-membered heterocyclyl comprising 1-4 heteroatoms selected from O, S(═O)ₚ, N and NR¹⁰ substituted with 0-3 R⁶ and 0-1 R⁷;
R⁶ is halo, ═O, —OH, —OC₁₋₄ alkyl, or C₁₋₄ alkyl substituted with 0-2 halo or OH;
R⁷ is C₁₋₃ alkyl substituted with 0-1 R⁸ and 0-1 R⁹, —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NRS(═O)ₚR^{c}, —OC(═O)R^{b}, —S(═O)ₚR^{c}, —S(═O)NR^{a}R^{a}, C₃₋₆ cycloalkyl, or a 4- to 6-membered heterocyclyl comprising 1-4 heteroatoms selected from O, S(═O)ₚ, N and NR^{d}, and substituted with 0-5 R^{e};
R⁸ is halo, —C(═O)OR^{b}, —C(═O)NHR^{a}, —C(═O)NHOR^{b}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}S(═O)ₚR^{c}, —NR^{a}S(O)ₚNR^{a}R^{a}, —OC(═O)NR^{a}R^{a}, —OC(═O)NR^{a}OR^{b}, —S(═O)ₚNR^{a}R^{a}, or —S(O)ₚR^{c};
R¹⁰ is H, C₁₋₄ alkyl substituted with 0-2 R¹¹, —C(═O)R^{b}, —C(═O)OR^{b}, —C(═O)NR^{a}R^{a}, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e};
R¹¹ is —OH, —C(═O)OH, or aryl;
R¹² is H, C₁₋₃ alkyl, or aryl;
R^{a} is H, C₁₋₅ alkyl substituted with 0-5 R^{e}, C₂₋₅ alkenyl substituted with 0-5 R^{e}, C₂₋₃ alkynyl substituted with 0-5 R^{e}, —(CH₂)ₙ—C₃₋₁₀carbocyclyl substituted with 0-5 R^{e}, or —(CH₂)ₙ-heterocyclyl substituted with 0-5 R^{e}: or R^{a} and R^{a} together with the nitrogen atom to which they are both attached form a heterocyclyl substituted with 0-5 R^{e};
R^{b} is H, C₁₋₅ alkyl substituted with 0-5 R^{e}, C₂₋₅ alkenyl substituted with 0-5 R^{e}, C₂₋₅ alkynyl substituted with 0-5 R^{e}, —(CH₂)ₙ—C₃₋₁₀carbocyclyl substituted with 0-5 R^{e}, or —(CH₂)ₙ-heterocyclyl substituted with 0-5 R^{e}:
R^{c} is C₁₋₅alkyl substituted with 0-5 R^{e}, C₂₋₅ alkenyl substituted with 0-5 R^{e}, C₂₋₅ alkynyl substituted with 0-5 R^{e}, C₃₋₆ carbocyclyl, or heterocyclyl:
R^{d} is H or C₁₋₄ alkyl;
R is halo, CN, ═O, C₁₋₅ alkyl substituted with 0-5 R^{g}, C₂₋₅ alkenyl substituted with 0-5 R^{g}, C₂₋₅ alkynyl substituted with 0-5 R^{g}, —(CH₂)ₙ—C₃₋₆ cycloalkyl, —(CH₂)ₙ-aryl, —(CH₂)ₙ-heterocyclyl, —(CH₂)ₙOR^{f}, or —C(═O)OR^{f};
R^{f} is H or C₁₋₃ alkyl,
R^{g} is halo, CN, OH, C₁₋₃ alkyl, or C₃₋₆ cycloalkyl;
n is zero, 1, 2, or 3; and
p is zero, 1, or 2.

11. The compound of claim 1, having Formula (VIII): 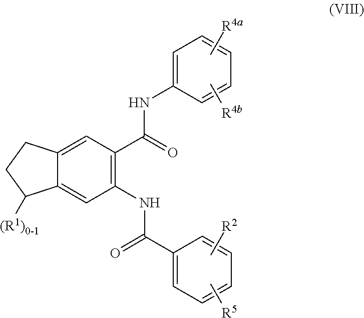
or a pharmaceutically acceptable salt thereof, wherein:
R¹ is ═O or —OH;
R² is halo, CN, —C(═O)OR^{b}, —NR^{a}R^{a}, C₁₋₄ alkyl substituted with 0-5 halo or OH, or —OC₁₋₄ alkyl substituted with 0-4 halo, OH, or —OC₁₋₄ alkyl;
R^{4a} is halo:
R^{4b} is C₁₋₄ alkyl substituted with 0-4 halo;
R⁵ is C₆ aryl substituted with 0-3 R⁶ and 0-2 R⁷, or a 3- to 12-membered heterocyclyl comprising 1-4 heteroatoms selected from O, S(═O)ₚ, N and NR¹⁰ substituted with 0-3 R⁶ and 0-1 R⁷;
R⁶ is halo, ═O, —OH, —OC₁₋₄ alkyl, or C₁₋₄ alkyl substituted with 0-2 halo or OH;
R⁷ is C₁₋₃ alkyl substituted with 0-1 Rand 0-1 R⁹, —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR³R^{a}, C₃₋₆ cycloalkyl, or a 4- to 6-membered heterocyclyl comprising 1-4 heteroatoms selected from O, S(═O)ₚ, N and NR^{d}, and substituted with 0-5 R^{e};
R⁸ is halo, —C(═O)OR^{b}, —C(═O)NHR^{a}, —C(═O)NHOR^{b}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}S(═O)ₚRc, —NR^{a}S(O)ₚNR^{a}R^{a}, —OC(═O)NR^{a}R^{a}, —OC(═O)NR^{a}OR^{b}, —S(═O)ₚNR^{a}R^{a}, or —S(O),
R¹⁰ is H, C₁₋₄ alkyl substituted with 0-2 R¹¹, —C(═O)R^{b}, —C(═O)OR^{b}, —C(═O)NR^{a}R^{a}, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{c};
R¹¹ is —OH, —C(═O)OH, or aryl:
R¹² is H, C₁₋₃ alkyl, or aryl;
R^{a} is H, C₁₋₅ alkyl substituted with 0-5 R^{e}, C₂₋₅ alkenyl substituted with 0-5 R^{e}, C₂₋₅ alkynyl substituted with 0-5 R^{e}, —(CH₂)ₙ—C₃₋₁₀ carbocyclyl substituted with 0-5 R^{e}, or —(CH₂)ₙ-heterocyclyl substituted with 0-5 R^{e}; or R^{a} and R^{a} together with the nitrogen atom to which they are both attached form a heterocyclyl substituted with 0-5 R^{e};
R^{b} is H, C₁₋₅ alkyl substituted with 0-5 R^{e}, C₂₋₅ alkenyl substituted with 0-5 R^{e}, C₂₋₅ alkynyl substituted with 0-5 R^{e}, —(CH₂)ₙ-C₃₋₁₀ carbocyclyl substituted with 0-5 R^{e}, or —(CH₂)ₙ-heterocyclyl substituted with 0-5 R^{e};
R^{c} is C₁₋₅ alkyl substituted with 0-5 R^{e}, C₂₋₅ alkenyl substituted with 0-5 R^{e}, C₂₋₅ alkynyl substituted with 0-5 R^{e}, C₃₋₆ carbocyclyl, or heterocyclyl;
R^{d} is H or C₁₋₄ alkyl;
R^{e} is halo, CN, ═O, C₁₋₅ alkyl substituted with 0-5 R^{g}, C₂₋₅ alkenyl substituted with 0-5 R^{g}, C₂₋₅ alkynyl substituted with 0-5 R^{g}, —(CH₂)ₙ—C₃₋₆ cycloalkyl, —(CH₂)ₙ-aryl, —(CH₂)ₙ-heterocyclyl, —(CH₂)ₙOR^{f}, or —C(═O)OR^{f};
R^{f} is H or C₁₋₃ alkyl,
R^{g} is halo, CN, OH, C₁₋₆ alkyl, or C₃₋₆ cycloalkyl;
n is zero, 1, 2, or 3; and
p is zero, 1, or 2.

12. The compound of claim 1, having Formula (IX): 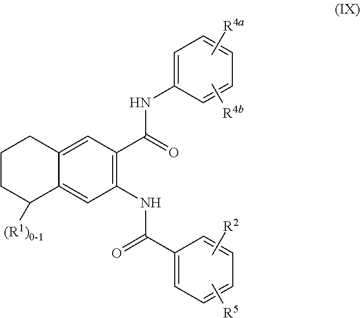
or a pharmaceutically acceptable salt thereof, wherein:
R¹ is ═O or —OH;
R² is halo, CN, —C(═O)OR^{b}, —NR^{a}R^{a}, C₁₋₄ alkyl substituted with 0-5 halo or OH or —OC₁₋₄ alkyl substituted with 0-4 halo, OH, or —OC₁₋₄ alkyl;
R^{4a} is halo;
R^{4b} is C₁₋₄ alkyl substituted with 0-4 halo;
R⁵ is C₆ aryl substituted with 0-3 R⁶ and 0-2 R⁷, or a 3- to 12-membered heterocyclyl comprising 1-4 heteroatoms selected from O, S(═O)ₚ, N and NR¹⁰ substituted with 0-3 R⁶ and 0-1 R⁷;
R⁶ is halo, ═O, —OH, —OC₁₋₄ alkyl, or C₁₋₄ alkyl substituted with 0-2 halo or OH;
R⁷ is C₁₋₃ alkyl substituted with 0-1 R⁸ and 0-1 R⁹, —OR^{b}, —NR^{a}R^{a}, —NR^{a}C(═O)R^{b}, —NR^{a}C(═O)OR^{b}, —NR^{a}C(═O)NR^{a}R^{a}, —NR^{a}S(═O)ₚR^{c}, —C(═O)R^{b}, —C(═O)OR^{b}, —C(═O)NR^{a}R^{a}, —C(═O)NR^{a}S(═O)ₚR^{c}, —OC(═O)R^{b}, —S(═O)ₚR^{c}, —S(═O)ₚNR^{a}R^{a}, C₃₋₆ cycloalkyl, or a 4- to 6-membered heterocyclyl comprising 1-4 heteroatoms selected from O, S(═O)ₚ, N and NR^{d}, and substituted with 0-5 R^{e};
R⁸ is halo, —C(═O)OR^{b}, —C(═O)NHR^{a}, —C(═O)NHOR^{b}, or C₁₋₄ alkyl substituted with 0-3 halo or OH;
R⁹ is —OR^{b}, —NR^{a}R^{a}, —NR^{a}C(═O)R^{b}, —NR^{a}C(═O)OR^{b}, —NR^{a}S(═O)ₚR^{c}, —NR^{a}S(O)ₚNR^{a}R^{a}, —OC(═O)NR^{a}R^{a}, —OC(═O)NR^{a}OR^{b}, —S(═O)ₚNR^{a}R^{a}, or —S(O)ₚR^{c};
R¹⁰ is H, C₁₋₄ alkyl substituted with 0-2 R¹¹, —C(═O)R^{b}, —C(═O)OR^{b}, —C(═O)NR^{a}R^{a}, C₃₋₆ cycloalkyl substituted with 0-5 R^{e}, or a 4- to 6-membered heterocyclyl comprising 1-4 heteroatoms selected from O, S(═O)ₚ, N and NR¹², and substituted with 0-5 R^{e};
R¹¹ is —OH, —C(═O)OH, or aryl;
R¹² is H, C₁₋₃ alkyl, or aryl;
R^{a} is H, C₁₋₅ alkyl substituted with 0-5 R^{e}, C₂₋₅ alkenyl substituted with 0-5 R^{e}, C₂₋₅ alkynyl substituted with 0-5 R^{e}, —(CH₂)ₙ-C₃₋₁₄ carbocyclyl substituted with 0-5 R^{e}, or —(CH₂)ₙ-heterocyclyl substituted with 0-5 R^{e}; or R^{a} and R^{a} together with the nitrogen atom to which they are both attached form a heterocyclyl substituted with 0-5 R^{e};
R^{b} is H, C₁₋₅ alkyl substituted with 0-5 R^{e}, C₂₋₅ alkenyl substituted with 0-5 R^{e}, C₂₋₅ alkynyl substituted with 0-5 R^{e}, —(CH₂)ₙ—C₃₋₁₀carbocyclyl substituted with 0-5 R^{e}, or —(CH₂)ₙ-heterocyclyl substituted with 0-5 R^{e};
R^{c} is C₁₋₅ alkyl substituted with 0-5 R^{e}, C₂₋₅ alkenyl substituted with 0-5 R^{e}, C₂₋₅ alkynyl substituted with 0-5 R^{e}, C₃₋₆ carbocyclyl, or heterocyclyl;
R^{d} is H or C₁₋₄ alkyl;
R^{e} is halo, CN, ═O, C₁₋₅ alkyl substituted with 0-5 R^{g}, C₂₋₅ alkenyl substituted with 0-5 R^{g}, C₂₋₅ alkynyl substituted with 0-5 R^{g}, —(CH₂)ₙ—C₃₋₆ cycloalkyl, —(CH₂)ₙ-4- to 6-membered heterocyclyl, —(CH₂)ₙ-aryl, —(CH₂)ₙ-heteroaryl, —(CH₂)ₙOR^{f}, or —C(═O)OR^{f};
R^{f} is H or C₁₋₃ alkyl,
R^{g} is halo, CN, OH, C₁₋₆ alkyl, or C₃₋₆ cycloalkyl;
n is zero, 1, 2, or 3; and
p is zero, 1, or 2.

13. A composition comprising a compound of claim 1, or a pharmaceutically acceptable salt thereof, and a pharmaceutically acceptable carrier.

14. A method for treating a disease associated with relaxin comprising administering a therapeutically effective amount of the composition of claim 13 to a patient in need thereof.

15. The method of claim 14 wherein the disease is selected from the group consisting of angina pectoris, unstable angina, myocardial infarction, heart failure, acute coronary disease, acute heart failure, chronic heart failure, and cardiac iatrogenic damage.

16. The method of claim 15 wherein the disease is heart failure.

17. The method of claim 14 wherein the disease is fibrosis.
